SEQUENCE LISTING (48 sequences)

SEQ ID NO: 1 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 2 - DNA, 339 bp, Homo sapiens
  source 1..339: mol_type = genomic DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 3 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 4 - DNA, 321 bp, Homo sapiens
  source 1..321: mol_type = genomic DNA
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 5 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 6 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 7 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 8 - protein, 93 aa, Homo sapiens
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 9 - protein, 21 aa, Homo sapiens
MALPVTALLLPLALLLHAARP

SEQ ID NO: 10 - protein, 15 aa, synthetic construct
  source 1..15: note = Description of Artificial Sequence: Synthetic peptide
GGGGSGGGGSGGGGS

SEQ ID NO: 11 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 12 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 13 - protein, 622 aa, Homo sapiens [sequence omitted]

SEQ ID NO: 14 - protein, 230 aa, synthetic construct
  source 1..230: note = Description of Artificial Sequence: Synthetic polypeptide
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGQAG

SEQ ID NO: 15 - DNA, 693 bp, synthetic construct
  source 1..693: mol_type = other DNA; note = Description of Artificial Sequence: Synthetic polynucleotide
caggtgcagctgcaggagtccggcccaggactggtgaagccttcggagaccctgtccctcacctgcactgtctctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagaggggaagaatggggcttttgatatctggggccaagggacaatggtcaccgtctcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactagtggccaggccggccac

SEQ ID NO: 16 - protein, 214 aa, synthetic construct
  source 1..214: note = Description of Artificial Sequence: Synthetic polypeptide
DIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 640 bp, synthetic construct
  source 1..640: mol_type = other DNA; note = Description of Artificial Sequence: Synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcagggttcagtggcagtggatctgggacagatttcactctcaccatcagcagtctgcaacctgaagattttgcaacttactactgtcaacagagttacagtaccccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctactgcctcagcagcaccctgacgctgagcaaagcagactacgagaaacacaaactctacgcctgcgaagtcacccatcagggcctgagctcgcccgtcacaaagagcttcaacaggggagagt

SEQ ID NO: 18 - protein, 214 aa, synthetic construct
  source 1..214: note = Description of Artificial Sequence: Synthetic polypeptide
RHQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 120 aa, synthetic construct
  source 1..120: note = Description of Artificial Sequence: Synthetic polypeptide
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSS

SEQ ID NO: 20 - protein, 262 aa, Homo sapiens
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSSGGGGSGGGGSGGGGSRHQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGQAGHHHHHHGDYKDDDDKG

SEQ ID NO: 21 - DNA, 801 bp, Homo sapiens
  source 1..801: mol_type = genomic DNA
atggccttaccagtgaccgccttgctcctgccgctggccttgctgctccacgccgccaggccgcaggtgcagctgcaggagtccggcccaggactggtgaagccttcggagaccctgtccctcacctgcactgtctctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagaggggaagaatggggcttttgatatctggggccaagggacaatggtcaccgtctcttcaggtggaggcggttcaggcggaggtggctctggcggtggcggatcacgacatcagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgctcactttcggcggagggaccaaggtggagatcaaacggactgcggccgca

SEQ ID NO: 22 - DNA, 801 bp, synthetic construct
  source 1..801: mol_type = other DNA; note = Description of Artificial Sequence: Synthetic polynucleotide
atggcgctgccggtgaccgcgctgctgctgccgctggcgctgctgctgcatgcggcgcgcccgcaggtgcagctgcaggaaagcggcccgggcctggtgaaaccgagcgaaaccctgagcctgacctgcaccgtgagcggcggcagcgtgagcagcggcagctattattggagctggattcgccagccgccgggcaaaggcctggaatggattggctatatttattatagcggcagcaccaactataacccgagcctgaaaagccgcgtgaccattagcgtggataccagcaaaaaccagtttagcctgaaactgagcagcgtgaccgcggcggataccgcggtgtattattgcgcgcgcgaaggcaaaaacggcgcgtttgatatttggggccagggcaccatggtgaccgtgagcagcggcggcggcggcagcggcggcggcggcagcggcggcggcggcagccgccatcagatgacccagagcccgagcagcctgagcgcgagcgtgggcgatcgcgtgaccattacctgccgcgcgagccagagcattagcagctatctgaactggtatcagcagaaaccgggcaaagcgccgaaactgctgatttatgcggcgagcagcctgcagagcggcgtgccgagccgctttagcggcagcggcagcggcaccgattttaccctgaccattagcagcctgcagccggaagattttgcgacctattattgccagcagagctatagcaccccgctgacctttggcggcggcaccaaagtggaaattaaacgcaccgcggcggcg

SEQ ID NO: 23 - DNA, 801 bp, synthetic construct
  source 1..801: mol_type = other DNA; note = Description of Artificial Sequence: Synthetic polynucleotide
atggccctcccggtaacggctctgctgcttccactcgcactgctcttgcatgctgccagaccacaggtccagctgcaggagagtgggcctggactggttaagccgagtgagacactttccttgacgtgcactgtgagcgggggaagtgtgtcctcaggtagttattactggtcctggattcgccagccaccaggaaagggactggagtggataggttatatctattattctggcagcactaattacaatccttctctcaaaagtagggtgacaatttcagtggatacttccaaaaatcagtttagtctgaagctcagctctgtgacagctgctgatactgcagtttactactgcgccagggaggggaagaatggcgccttcgatatttggggacagggcactatggtgactgtatcaagcggaggcggtggcagcggcgggggagggagtggaggcggcgggtctcgacatcagatgacacagagcccatcatcacttagcgccagcgttggcgaccgggttacgataacatgcagggcttcccaatctatcagttcttatctgaactggtatcagcagaaaccaggtaaggcccccaagctgctcatctacgcagcctcatccctgcagagcggcgtccctagtcgattttccggtagtgggtcagggacagattttaccctgactatcagttcactgcagcccgaggacttcgcgacatactattgccaacagtcctatagtacacccttgacatttggcggcgggactaaagtagaaattaaacgcaccgcggccgca

SEQ ID NO: 24 - protein, 10 aa, synthetic construct
  source 1..10: note = Description of Artificial Sequence: Synthetic peptide
GGSVSSGSYY

SEQ ID NO: 25 - protein, 7 aa, synthetic construct
  source 1..7: note = Description of Artificial Sequence: Synthetic peptide
IYYSGST

SEQ ID NO: 26 - protein, 12 aa, synthetic construct
  source 1..12: note = Description of Artificial Sequence: Synthetic peptide
AREGKNGAFDIW

SEQ ID NO: 27 - protein, 6 aa, synthetic construct
  source 1..6: note = Description of Artificial Sequence: Synthetic peptide
QSISSY

SEQ ID NO: 28 - protein, 4 aa, synthetic construct
  source 1..4: note = Description of Artificial Sequence: Synthetic peptide
AASS

SEQ ID NO: 29 - protein, 10 aa, synthetic construct
  source 1..10: note = Description of Artificial Sequence: Synthetic peptide
QQSYSTPLTF

SEQ ID NO: 30 - protein, 21 aa, Homo sapiens
MALPVTALLLPLALLLHAARP

SEQ ID NO: 31 - protein, 21 aa, Homo sapiens
MALPVTALLLPLALLLHAARP

SEQ ID NO: 32 - protein, 21 aa, Homo sapiens
MALPVTALLLPLALLLHAARP

SEQ ID NO: 33 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 34 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 35 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYSLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 36 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 37 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 38 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 39 - protein, 287 aa, Homo sapiens
MSPHPTALLGLVLCLAQTIHTQEEDLPRPSISAEPGTVIPLGSHVTFVCRGPVGVQTFRLERDSRSTYNDTEDVSQASPSESEARFRIDSVREGNAGLYRCIYYKPPKWSEQSDYLELLVKESSGGPDSPDTEPGSSAGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 40 - protein, 370 aa, Homo sapiens
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 41 - protein, 181 aa, Homo sapiens
MLLEPGRGCCALAILLAIVDIQSGGCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSSGFLQEKVWVMLVTSLVALQAL

SEQ ID NO: 42 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 43 - protein, 244 aa, synthetic construct
  source 1..244: note = Description of Artificial Sequence: Synthetic polypeptide
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQAAAPTTTPAPRPPTPAPTIASQPLSLRPEACRPAAGGAVHTRGLDFACDIYIWAPLAGTCGVLLLSLVITLYCNHRRIQ

SEQ ID NO: 44 - protein, 23 aa, synthetic construct
  source 1..23: note = Description of Artificial Sequence: Synthetic peptide
GSGATNFSLLKQAGDVEENPGPM

SEQ ID NO: 45 - protein, 236 aa, synthetic construct
  source 1..236: note = Description of Artificial Sequence: Synthetic polypeptide
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 46 - protein, 502 aa, synthetic construct
  source 1..502: note = Description of Artificial Sequence: Synthetic polypeptide
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQAAAPTTTPAPRPPTPAPTIASQPLSLRPEACRPAAGGAVHTRGLDFACDIYIWAPLAGTCGVLLLSLVITLYCNHRRIQGSGATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 47 - DNA, 1726 bp, synthetic construct
  source 1..1726: mol_type = other DNA; note = Description of Artificial Sequence: Synthetic polynucleotide
accggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactggccaccatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccaggcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacaggcggatccaaggatctggagcaacaaacttctcactactcaaacaagcaggtgacgtggaggagaatcccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgag

SEQ ID NO: 48 - protein, 5 aa, synthetic construct
  source 1..5: note = Description of Artificial Sequence: Synthetic peptide
SGGGS

(19) United States
(12) Patent Application Publication
(10) Pub. No.: US 2025/0034228 A1
(43) Pub. Date: Jan. 30, 2025

(54) IMMUNE CELL COMPOSITIONS AND METHODS OF USE

(71) Applicant: Memorial Sloan Kettering Cancer Center, New York, NY (US)

(72) Inventors: Prasad S. Adusumilli, New York, NY (US); Michel Sadelain, New York, NY (US)

(73) Assignee: Memorial Sloan Kettering Cancer Center, New York, NY (US)

(21) Appl. No.: 18/908,558

(22) Filed: Oct. 7, 2024

Related U.S. Application Data
(62) Division of application No. 17/557,607, filed on Dec. 21, 2021, now Pat. No. 12,139,524.
Application No. 17/557,607 is a division of application No. 15/757,276, filed on Mar. 2, 2018, now Pat. No. 11,242,375.
(60) Provisional application No. 62/214,809, filed on Sep. 4, 2015.

(51) Int. Cl. C07K 14/725; A61K 35/28; A61K 38/00; A61K 38/17; A61K 38/19; A61K 38/20; A61K 38/48; A61K 39/00; A61K 39/395; A61P 35/00; C07K 14/705; C07K 16/30; C12N 5/0783; C12N 9/64

(52) U.S. Cl. CPC C07K 14/7051; A61K 35/28; A61K 38/177; A61K 38/1774; A61K 38/193; A61K 38/2013; A61K 38/4873; A61K 39/39558; A61K 39/4611; A61K 39/4631; A61K 39/464402; A61K 39/464411; A61K 39/464468; A61K 39/464495; A61K 2239/38; A61K 2239/46; A61K 2239/55; A61P 35/00; C07K 14/70503; C07K 14/70517; C07K 14/70521; C07K 14/70578; C07K 16/30; C07K 16/3069; C12N 5/0636; C12N 5/0638; C12N 9/6472; C12Y 304/22062; A61K 38/00; C07K 2317/622; C07K 2319/02; C07K 2319/03; C07K 2319/33; C07K 2319/74; C12N 2510/00

(57) ABSTRACT

Disclosed herein are cells that are immune cells or precursor cells thereof, which cells recombinantly express a chimeric antigen receptor (CAR), and a dominant negative form of an inhibitor of a cell-mediated immune response of the immune cell, wherein the CAR binds to a cancer antigen. Also disclosed herein are T cells that recognize and are sensitized to a cancer antigen, which T cells recombinantly express a dominant negative form of an inhibitor of a T cell-mediated immune response. Additionally provided are methods of using such cells to treat cancer in a subject in need thereof.

### 1. CROSS-REFERENCE TO RELATED APPLICATIONS

This application claims the benefit of U.S. Provisional application No. 62/214,809, filed Sep. 4, 2015, which is incorporated by reference herein in its entirety.

### GOVERNMENT RIGHTS STATEMENT

This invention was made with government support under grant numbers W81XWH-11-1-0783 and W81XWH-12-1-0230, awarded by the U.S. Department of Defense. The government has certain rights in the invention.

### REFERENCE TO SEQUENCE LISTING SUBMITTED ELECTRONICALLY

This application incorporates by reference a Sequence Listing with this application as an ASCII text file entitled “13542-020-228_SL.TXT” created on Aug. 30, 2016 and having a size of 81,752 bytes.

### 2. FIELD

The present invention relates generally to cancer treatment, and more specifically to immunotherapy for cancer treatment. 3. BACKGROUND

Recent years have provided tremendous advancements in the treatment of cancer. Among these advancement are the use of immunotherapy, where a cancer patient's immune response is harnessed to treat cancer. Such immunotherapy treatment methods include the use of cell-based immunotherapy, where cells of the immune system are utilized for therapeutic treatment. Immune system cells such as T cells and other immune cells can be modified to target tumor antigens.

In response to immune attack, solid tumors upregulate PD-L1 in response to immune attack, which in turn binds PD-1 receptor expressed on T cells, resulting in T-cell inhibition (see Pardoll, Nat. Rev. Cancer 12(4):252-64 (2012)). Upregulation of PD-L1 on T cells and antigen presenting cells (APCs) was described as well, resulting in inhibition of activated T cells (Talay et al., Proc. Natl. Acad. Sci. USA 106(8):2741-2746 (2009); Latchman et al., Proc. Natl. Acad. Sci. USA 101(29):10691-10696 (2004); Liu et al., J. Cell. Mol. Med. 19(6):1223-1233 (2015)). PD-1/PD-L1 checkpoint blockade therapy counteracts this inhibition, thereby leading to activated T cells. Various strategies to inhibit the immune checkpoint blockade mediated by PD-1 have been described, including the use of PD-1 or PDL-1 antibodies (Burga et al., Cancer Immunol. Immunother. 64(7):817-829 (2015); Moon et al., Clin. Cancer Res. 20(16):4262-4273 (2014); John et al., Clin. Cancer Res. 19(20):5636-5646 (2013)), RNA interference (Borkner et al., CancerImmunol. Immunother. 59(8):1173-1183 (2010)), and co-stimulatory molecules (Prosser et al., Mol. Immunol. 51(3-4):263-272 (2012); Ankri et al., J. Immunol. 191(8):4121-4129 (2013)).

Chimeric antigen receptors (CARs) are synthetic receptors that retarget T cells to tumor surface antigens (Sadelain et al., Nat. Rev. Cancer. 3(1):35-45 (2003); Sadelain et al., Cancer Discovery 3(4):388-398 (2013)). Chimeric antigen receptors (CARs) are engineered receptors that provide both antigen binding and immune cell activation functions. CARs can be used to graft the specificity of an antibody, such as a monoclonal antibody, onto an immune cell such as a T cell. First-generation receptors link an antibody-derived tumor-binding element, such as an scFv, that is responsible for antigen recognition to either CD3zeta or Fc receptor signaling domains, which trigger T-cell activation. The advent of second-generation CARs, which combine activating and costimulatory signaling domains, has led to encouraging results in patients with chemorefractory B-cell malignancies (Brentjens et al., Science Translational Medicine 5(177):177ra38 (2013); Brentjens et al., Blood 118(18):4817-4828 (2011); Davila et al., Science Translational Medicine 6(224):224ra25 (2014); Grupp et al., N. Engl. J. Med. 368(16):1509-1518 (2013); Kalos et al., Science Ihanslational Medicine 3(95):95ra73 (2011)). The translation of this clinical success to solid tumors requires overcoming additional obstacles, including achieving sufficient T-cell infiltration into tumors and resisting tumor immune escape. The extracellular antigen-binding domain of a CAR is usually derived from a monoclonal antibody (mAb) or from receptors or their ligands. Antigen recognition is therefore not MHC-restricted (Riviere et al., Curr. Hematol. Rep. 3:290-297 (2004); Stephan et al., Nat. Med. 13:1440-1449 (2007)) and is therefore applicable to any patient expressing the target cancer antigen using the corresponding CAR. Antigen binding by the CARs triggers phosphorylation of immunoreceptor tyrosine-based activation motifs (ITAMs) in the intracellular domain, initiating a signaling cascade required for cytolysis induction, cytokine secretion, and proliferation. Because MHC restriction of antigen recognition is bypassed, the function of CAR-targeted T cells is not affected by HLA downregulation or defects in the antigen-processing machinery.

To overcome the limitations of tumor infiltration and delayed activation observed with systemic T-cell administration, the merits of regional administration of mesothelin-specific CAR T cells in a clinically relevant model of pleural mesothelioma was recently demonstrated (Adusumilli et al., Science-Translational Medicine 6(261):261ra151 (2014)). Mesothelin (MSLN) is a tumor-associated cell-surface antigen, which was selected on the basis of its overexpression in several cancers and observations of its association with tumor aggressiveness and decreased survival in mesothelioma, lung and breast cancer patients (Argani et al., Clin. Cancer Res. 7(12):3862-3868 (2001); Frierson et al., Hum. Pathol. 34(6):605-609 (2003); Gubbels et al., Mol. Cancer 5(1):50 (2006); Kachala et al., Clin. Cancer Res. 20(4):1020-1028 (2014); Li et al., Mol. Cancer Ther. 7(2):286-296 (2008); Rizk et al., Cancer Epidemiol Biomarkers Prev. 21(3):482-486 (2012); Servais et al., Clin. Cancer Res. 18(9):2478-2489 (2012); Tozbikian et al., PLoS One 9(12):el 14900 (2014)). Regional administration ofMSLN-targeted CAR T cells eradicates primary tumor and establishes long-term systemic immunosurveillance at 30-fold lower doses than intravenous administration (Adusumilli et al., Science Translational Medicine 6(261):261ra151 (2014)). These results are encouraging for the treatment of solid malignancies and have led to the initiation of a phase I clinical trial of intrapleural administration of mesothelin-targeted CAR T cells (ClinicalTrials.gov record NCT02414269).

To eliminate tumor cells, T cells must sustain cytolytic and proliferative function first in the absence of costimulatory ligands on tumor cells and elude the eventual inhibitory signals in the tumor microenvironment upon repeated antigen encounter. The success of second generation CAR T cells has been attributed to the enhanced T-cell persistence observed with costimulatory signaling domains, such as CD28 and 4-1BB. However, T cells naturally undergo activation-induced upregulation of coinhibitory pathways, which may limit the antitumor immune response. PD-1, CTLA-4, and other coinhibitory receptors are upregulated in T cells following antigen encounter, while tumor cells augment the expression of coinhibitory ligands following exposure to T-cell-secreted Th1 cytokines (McGray et al., Mol. Ther. 22(1):206-218 (2014); Spranger et al., Science Translational Medicine 5(200):200ra116 (2013); Moon et al., Clin. Cancer Res. 20(16):4262-4273 (2014)). The success of antibody therapy targeting immune checkpoints such as PD-1 and CTLA-4 underscores the therapeutic potential of immunotherapies that aim to counteract immune inhibition (Hodi et al., N. Engl. J Med. 363(8):711-723 (2010); Wolchok et al., N. Engl. J. Med. 369(2):122-133 (2013); Topalian et al., N. Engl. J. Med. 366(26):2443-2454 (2012)). However, success with antibody therapies require the presence of infiltrating T cells and a relatively high mutation burden (Ji et al., Cancer Immunol. Immunother. 61(7):1019-1031 (2012); Rizvi et al., Science 348(6230):124-128 (2015); Hamid et al., J. Translational Med.;9(204) doi: 10.1186/1479-5876-9-204 (2011)). Adoptive transfer of tumor-targeted T cells can therefore fill the void in patients with less immunogenic or “noninflamed” tumors (Nesbeth et al., J. Immunol. 184(10):5654-5662 (2010); Spear et al., Oncoimmunology 2(4):e23564 (2013)). As adoptively transferred T cells are themselves susceptible to immuno inhibition, strategies to counteract immuno inhibition using antibodies have been described (John et al., Clin. Cancer Res. 19(20):5636-5646 (2013); Strome et al., Cancer Res. 63(19):6501-6505 (2003)).

While immunotherapy methods have provided new modalities for cancer treatment, including antibody therapies and cell-based therapies using immune cells such as T cells, limitations have been found for the effectiveness of such treatments. Malignant cells adapt to generate an immunosuppressive microenvironment that protects the cells from immune recognition and elimination. This tumor microenvironment poses a challenge to methods of treatment involving stimulation of an immune response, including immunotherapy methods such as targeted T cell therapies. Solid tumors can be restricted within anatomical compartments such that access of therapeutic immune cells to the tumors is limited. In addition, an immunosuppressive microenvironment must be overcome so that the immunotherapy is effective. The successful elimination of solid tumors or other cancers thus requires effective tumor infiltration and overcoming tumor-induced or cancer cell-induced immunosuppression.

Thus, there exists a need for therapies to provide improved treatment of cancer that overcome microenvironments associated with malignant cells or tumors that inhibit effective immunotherapies. The present invention satisfies this need and provides related advantages as well.

### 4. SUMMARY OF INVENTION

The present invention relates to cells that are immune cells or precursor cells thereof, which recombinantly express a chimeric antigen receptor (CAR), and a dominant negative form of an inhibitor of a cell-mediated immune response.

In one aspect, provided herein is a cell that is an immune cell or precursor cell thereof, which cell recombinantly expresses (a) a chimeric antigen receptor (CAR), and (b) a dominant negative form of an inhibitor of a cell-mediated immune response of the immune cell, wherein the CAR binds to a cancer antigen. In certain embodiments, the immune cell is a T cell. In certain embodiments, the precursor cell is a hematopoietic stem or hematopoietic progenitor cell. In a specific embodiment, the immune cell is a cytotoxic T lymphocyte (CTL). In another embodiment, the cell is a T cell. In another embodiment, the cell is a Natural Killer (NK) cell.

In certain embodiments of a cell of the invention, the inhibitor of a cell-mediated immune response is an immune checkpoint inhibitor. In certain embodiments, the immune checkpoint inhibitor is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160. In a particular embodiment, the immune checkpoint inhibitor is PD-1. In another embodiment, the inhibitor of a cell-mediated immune response is transforming growth factor β (TGF-β) receptor.

In certain embodiments of cells of the invention, the cancer antigen is selected from the group consisting of mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-α and β (FRα and β), Ganglioside G2 (GD2), Ganglioside G3 (GD3), human Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Rα2), κ-light chain, kinase insert domain receptor (KDR), Lewis A (CA19.9), Lewis Y (LeY), L1 cell adhesion molecule (LlCAM), melanoma-associated antigen 1 (melanoma antigen family A1, MAGE-A1), Mucin 16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-1H, HERK-V, IL-11Rα, Latent Membrane Protein 1 (LMP1), Neural cell-adhesion molecule (N-CAM/CD56), and Trail Receptor (TRAIL R). In a particular embodiment, the cancer antigen is mesothelin. In a particular embodiment, the cancer antigen is mesothelin and the inhibitor of a cell-mediated immune response is PD-1. In certain embodiments of the invention, the cell further recombinantly expresses a suicide gene. In a specific embodiment, the suicide gene comprises inducible Caspase 9.

In another aspect, provided herein are pharmaceutical compositions comprising a therapeutically effective amount of a cell of the invention that is an immune cell or precursor cell thereof, which cell recombinantly expresses (a) a chimeric antigen receptor (CAR), and (b) a dominant negative form of an inhibitor of a cell-mediated immune response of the immune cell, wherein the CAR binds to a cancer antigen; and a pharmaceutically acceptable carrier.

In yet another aspect, provided herein are polypeptides comprising (a) at least a portion of an extracellular domain of an immune checkpoint inhibitor, said portion comprising the ligand binding region, and (b) a transmembrane domain; wherein the polypeptide is a dominant negative form of the immune checkpoint inhibitor. In certain embodiments, the transmembrane domain is derived from a polypeptide other than the immune checkpoint inhibitor. In certain embodiments, the polypeptide lacks the intracellular domain of the polypeptide.

In certain embodiments of a polypeptide of the invention, the immune checkpoint inhibitor is a receptor selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160. In a specific embodiment, the immune checkpoint inhibitor is PD-1.

In certain embodiments of a polypeptide of the invention, the transmembrane domain is of a cell surface polypeptide of a T cell. In specific embodiments, the transmembrane domain is of a cell surface polypeptide selected from the group consisting of CD3, CD4, CD8, CD28, 4-1BB, OX40, ICOS, CTLA-4, LAG3, 2B4 and BTLA. In specific embodiments, the transmembrane domain is of the cell surface polypeptide is CD8 or CD28. In specific embodiments of a polypeptide of the invention, the amino acid sequence of the polypeptide consists of the extracellular domain of PD-1 fused to the transmembrane and hinge domains of CD8.

In another aspect, provided herein are nucleic acids encoding the polypeptides of the invention encoding a dominant negative form of an immune checkpoint inhibitor, wherein the dominant negative form is a polypeptide comprising (a) at least a portion of an extracellular domain of an immune checkpoint inhibitor, said portion comprising the ligand binding region, and (b) a transmembrane domain; wherein the polypeptide is a dominant negative form of the immune checkpoint inhibitor. In still another aspect, provided herein are vectors comprising the nucleic acid. In yet another aspect, provided herein are cells comprising the polypeptide of the invention encoding a dominant negative form of an immune checkpoint inhibitor, described above. In another aspect, provided herein are cells comprising the nucleic acid of the invention, described above. In another embodiment, provided herein are cells comprising a vector, which comprises a nucleic acid of the invention, described above.

In another aspect, provided herein are T cells that recognize and are sensitized to a cancer antigen, which T cells recombinantly express a dominant negative form of an inhibitor of a T cell-mediated immune response. In certain embodiments of T cells of the invention, the inhibitor of a T cell-mediated immune response is an immune checkpoint inhibitor. In a particular embodiment, the immune checkpoint inhibitor is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160. In a specific embodiment, the immune checkpoint inhibitor is PD-1. In another embodiment, the inhibitor of a cell-mediated immune response is transforming growth factor β (TGF-β) receptor.

In certain embodiments of T cells of the invention, the cancer antigen is selected from the group consisting of mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-α and β (FRα and β), Ganglioside G2 (GD2), Ganglioside G3 (GD3), human Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Rα2), κ-light chain, kinase insert domain receptor (KDR), Lewis A (CA19.9), Lewis Y (LeY), L1 cell adhesion molecule (LlCAM), melanoma-associated antigen 1 (melanoma antigen family A1, MAGE-A1), Mucin 16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-1H, HERK-V, IL-11Rα, Latent Membrane Protein 1 (LMP1), Neural cell-adhesion molecule (N-CAM/CD56), and Trail Receptor (TRAIL R). In a particular embodiment, the cancer antigen is mesothelin. In another particular embodiment, the cancer antigen is mesothelin and the inhibitor of a cell-mediated immune response is PD-1.

In certain embodiments of T cells of the invention, the T cell further recombinantly expresses a suicide gene. In a particular embodiment, the suicide gene comprises inducible Caspase 9.

In still another aspect, provided herein is a pharmaceutical composition comprising a therapeutically effective amount of the T cells described above that recognize and are sensitized to a cancer antigen and which recombinantly express a dominant negative form of an inhibitor of a T cell-mediated immune response; and a pharmaceutically acceptable carrier.

In another aspect, provided herein are methods of treating a cancer in a subject in need thereof, comprising administering to the subject a therapeutically effective amount of a cell that is an immune cell or precursor cell thereof, which cell recombinantly expresses (a) a chimeric antigen receptor (CAR), and (b) a dominant negative form of an inhibitor of a cell-mediated immune response of the immune cell, wherein the CAR binds to a cancer antigen, described above.

In another aspect, provided herein are methods of treating a cancer in a subject in need thereof, comprising administering to the subject a pharmaceutical composition comprising a cell that is an immune cell or precursor cell thereof, which cell recombinantly expresses (a) a chimeric antigen receptor (CAR), and (b) a dominant negative form of an inhibitor of a cell-mediated immune response of the immune cell, wherein the cancer antigen is an antigen of the cancer. In another aspect, provided herein are methods of treating a cancer in a subject in need thereof, comprising administering to the subject a pharmaceutical composition comprising T cells that recognize and are sensitized to a cancer antigen and which recombinantly express a dominant negative form of an inhibitor of a T cell-mediated immune response, wherein the CAR binds to a cancer antigen.

In another aspect, provided herein are methods of treating a cancer in a subject in need thereof, comprising administering to the subject a therapeutically effective amount of T cells that recognize and are sensitized to a cancer antigen and which recombinantly express a dominant negative form of an inhibitor of a T cell-mediated immune response, wherein the cancer antigen is an antigen of the cancer.

In certain aspects of methods of the invention, the cancer is selected from the group consisting of mesothelioma, lung cancer, pancreatic cancer, ovarian cancer, breast cancer, colon cancer, pleural tumor, glioblastoma, esophageal cancer, gastric cancer, and synovial sarcoma. In certain aspects of methods of the invention, the administering is by intrapleural administration, intravenous administration, subcutaneous administration, intranodal administration, intratumoral administration, intrathecal administration, intraperitoneal administration, intracranial administration, or direct administration to the thymus.

In certain aspects of methods of the invention, the cancer antigen is mesothelin, and the cancer is selected from the group consisting of mesothelioma, lung cancer, and breast cancer. In specific embodiments of methods of the invention, the subject has malignant pleural disease. In specific embodiments of methods of the invention, the cells are administered intrapleurally. In certain aspects of methods of the invention, the subject has a tumor. In certain aspects of methods of the invention, tumor growth is inhibited.

In certain aspects of methods of the invention, the cell is administered in a dose in the range of 10⁴ to 10¹⁰ cells per kilogram of body weight. In specific embodiments, the dose is in the range of 3×10⁵ to 3×10⁶ cells per kilogram of body weight.

In certain aspects of methods of the invention, the subject is a human. In specific aspects, a cell of the invention that is an immune cell or precursor cell thereof, is derived from a human, which cell recombinantly expresses (a) a chimeric antigen receptor (CAR), and (b) a dominant negative form of an inhibitor of a cell-mediated immune response of the immune cell, wherein the CAR binds to a cancer antigen. In specific aspects, T cells of the invention that recognize and are sensitized to a cancer antigen, which T cells recombinantly express a dominant negative form of an inhibitor of a T cell-mediated immune response, are T cells derived from a human. In specific aspects of methods of the invention, the cells used in the methods to treat a human subject are derived from a human.

In certain aspects of methods of the invention, the CAR comprises a co-stimulatory signaling domain. In certain embodiments, the co-stimulatory signaling domain is the intracellular signaling domain of 4-1BB. In certain embodiments, the method of the invention further comprises administering a cytokine to the subject. In certain embodiments, the cytokine is IL-2 or GM-CSF. In a particular embodiment the cytokine is IL-2.

In certain aspects of methods of the invention, the method further comprises administering an immune cell recombinantly expressing the chimeric antigen receptor (CAR) and a switch receptor, wherein the switch receptor comprises (i) at least the extracellular ligand binding domain of an immune checkpoint inhibitor, (ii) a transmembrane domain, and (iii) a co-stimulatory signaling domain. In certain embodiments, the co-stimulatory signaling domain of the switch receptor is different from the co-stimulatory signaling domain of the CAR. In certain embodiments, the co-stimulatory signaling domain of the CAR is the intracellular signaling domain of CD28. In a specific embodiment, the co-stimulatory signaling domain of the switch receptor is the intracellular signaling domain of 4-1BB.

### 5. DESCRIPTION OF THE DRAWINGS

FIGS. 1A-1E show that chimeric antigen receptors (CARs) with CD28 or 4-1BB costimulation exhibit equivalent effector cytokine secretion and proliferation in vitro upon initial antigen stimulation. FIG. 1A. First- and second-generation CARs. FIG. 1B. Mesothelin (MSLN)-targeted CARs contain the CD3ζ endodomain either alone (Mz, first-generation CAR) or in combination with the CD28 (M28z) or 4-1BB (MBBz) costimulatory domain (second-generation CAR). A prostate-specific membrane antigen (PSMA)-directed CAR with CD28 costimulation (P28z) as well as PSMA-expressing targets (PSMA+) are included in experiments as negative controls. CYT, cytoplasmic domain; LS, leader sequence; LTR, long terminal repeat; SA, splice acceptor; SD, splice donor; TM, transmembrane. FIGS. 1C-1E. Antigen-specific effector functions of CAR-transduced T cells. FIG. 1C. Lysis of MSLN-expressing targets (MSLN+), but not PSMA+ targets, as measured by chromium-release assays. FIG. 1D. 4-1BB and CD28 costimulations enhance cytokine secretion, as assessed by Luminex assay, after coculture of CAR T cells with MSLN+ cells. FIG. 1E. M28z and MBBz CARs facilitate robust T-cell accumulation after stimulation with MSLN+ cells. Data represent the mean±SEM (FIGS. 1C, 1E) of three replicates or are plotted as individual points (FIG. 1D). ***P<0.001, comparing costimulated CAR T cells (M28z or MBBz) with the first-generation receptor (Mz), by Student's/test; significance was determined using the Bonferroni correction for multiple comparisons.

FIGS. 2A-2C show that mice treated with M28z and MBBz CAR T cells demonstrate tumor eradication at a higher dose whereas treatment with lower doses results in higher rate of tumor relapse with M28z. FIG. 2A. In vivo bioluminescence imaging (BLI) was used to monitor tumor burden (firefly luciferase+ MSLN+) in NOD/SCID/γc^{null} mice. Mice with established pleural tumor were treated with a single dose of le5 (effector to target (E:T) ratio 1:3,000), 8e4 (E:T1:3,750), or 5e4 (E:T 1:6,000) M28z or MBBz CAR T cells. The (†) symbol indicates the death of a mouse. Two similar experiments with the same donor are combined for the illustration. FIG. 2B. Mice were treated with 4e4 CAR T cells (E:T 1:7,500). The first generation Mz CAR and negative control P28z are included. FIG. 2C. Kaplan-Meier survival analysis comparing the in vivo efficacy of intrapleural administration of 4e4 Mz (n=13, second curve from left), M28z (n=15, third curve from left), MBBz (n=8, curve across top), and P28z (n=3, first curve from left) CAR T cells. Median survival in days following T-cell administration (P28z, 16; Mz, 45; M28z, 64; MBBz, not reached). The survival curve was analyzed using the log-rank test. *P<0.05; **P<0.01.

FIGS. 3A-3C show that M28z- and MBBz-treated mice demonstrate similar early and long-term CAR T-cell accumulation, and M28z-treated mice with progressing tumors contain persisting CAR T cells. FIG. 3A. CD28 and 4-1BB costimulation enhance intratumoral CAR T-cell accumulation to equal extents. The left panels show the results of tumor BLI after after administration of a single dose of 8e⁴ CAR T cells. After 6 days, T cells were harvested from the tumor; x denotes mice whose T-cell counts are represented as data points. The right panel shows absolute CAR T cells per gram of tumor tissue (*P<0.05). Student's t tests were performed and statistical significance was determined using the Bonferroni correction for multiple comparisons. FIG. 3B. CD28 and 4-1BB costimulation enhance CAR T-cell persistence, as measured in the spleen, to equal extents. Absolute CAR T cells per spleen are shown 73 days after intrapleural administration of CAR T cells (8e⁴). The left panels show the results of tumor BLI; x denotes mice whose T-cell counts are represented as data points (*P<0.05). Student's t tests were performed and statistical significance was determined using the Bonferroni correction for multiple comparisons.

FIG. 3C. Mice treated with a low dose of M28z T cells (4e⁴) display tumor recurrence with persisting CAR T cells in the spleen and tumor. The left panel shows the results of tumor BLI. Spleen and tumor from mice denoted by an x were harvested and used for FACs analysis (middle panel) and T-cell quantification (right panel).

FIGS. 4A-4D show that CAR T cells become exhausted following in vivo antigen exposure, although MBBz CAR T cells preferentially retain effector cytokine secretion and cytotoxicity. FIG. 4A. Six days after intrapleural administration of CAR T cells, M28z and MBBz CAR T cells were isolated from the tumor and spleen and subjected to ex vivo antigen stimulation. FIG. 4B. Chromium-release assay upon ex vivo stimulation demonstrates a decrease in M28z but persistent MBBz cytolytic function (E:T ratio 1:5). FIG. 4C. Cytokine secretion measurements demonstrate decreases in effector cytokine secretion by CAR T cells, although MBBz CAR T cells are better able to retain secretion. FIG. 4D. RT-PCR measurements of GzB, IFN-γ, and IL-2 expression by harvested CAR T cells correlate well with protein level measurements. Data represent the fold-change relative to the mRNA expression of unstimulated M28z CAR T cell in vitro. Data represent the mean + SEM of three individual wells per condition. Student's t tests were performed, and statistical significance was determined using the Bonferroni correction for multiple comparisons (*P<0.05; **P<0.01; ***P<0.001). Results are reproduced in two separate cohorts of mice used for each of the two experiments. In each of FIGS. 4B-4D, each pair of bar graphs show, from left to right, M28z, MBBZ.

FIGS. 5A-5E show that CAR T cells become exhausted upon repeated antigen stimulation in vitro, although MBBz CAR T cells preferentially retain effector cytokine secretion and cytotoxicity in vitro and upon tumor rechallenge in vivo. FIG. 5A. Both M28z and MBBz CAR T cells retain proliferative capacity in vitro upon repeated antigen stimulation. T cells were also tested for cytotoxicity by chromium-release assay and for cytokine secretion by Luminex assay (FIGS. 5B-5D). FIG. 5B. CAR T cells demonstrate equal killing at the first stimulation (left) and loss of cytolytic function upon repeated antigen stimulation, although MBBz CAR T cells are better able to retain cytolytic function as measured by chromium-release assay (circles, MZ; triangles, M28z; diamonds, MBBz). FIG. 5C. Cytotoxic granule release as measured by CD107a expression (shown at the third stimulation) correlates with chromium release assay (FIG. 5B). Data represent the mean±SD (triplicates) of the fold-change relative to the CD107a MFI of unstimulated CAR T cells (each pair of bar graphs shows, from left to right, M28z, MBBz). FIG. 5D. Cytokine secretion measurements similarly demonstrate loss of CAR T-cell effector function upon repeated antigen encounter; again, MBBz CAR T cells are better able to preserve their function (each set of symbols above “Stim 1,” “Stim 2” and “Stim 3” are, from left to right, Mz, M28z, MBBz). FIG. 5E. Although equally persistent, MBBz CAR T cells demonstrate superior functional persistence. Twenty-eight days after pleural tumor eradication (following a single dose of 1e⁵ CAR T cells), 1e⁶ MSLN+ tumor cells were injected into the pleural cavity (tumor rechallenge). MBBz CAR T cells prevented tumor growth in all mice, whereas tumor growth and death were observed in 2 of 4 mice initially treated with M28z CAR T cells. Student's/tests were performed and statistical significance was determined using the Bonferroni correction (*P<0.05; ***P<0.001). Data represent the mean±SEM of three replicates or are plotted as individual points.

FIGS. 6A-6F show that PD-1 receptor and its ligands are upregulated in vivo (FIGS. 6A-6D, harvested T cells; FIGS. 6E-6F, tumor cells). FIG. 6A. Tumor-infiltrating M28z and MBBz CAR T cells express inhibitory receptors 6 days after their administration, but MBBz CAR T cells express lower levels of PD-1. FIG. 6B. Mean fluorescence intensity (MFI) of PD-1 receptor expression of tumor-infiltrating CAR T cells (TIL) 6 days after intrapleural administration. FIG. 6C. Relative expression of PD-1 mRNA in CD4 and CD8 subsets of tumor-infiltrating CAR T cells 6 days after intrapleural administration. Data are represented in fold-change relative to the PD-1 mRNA expression of unstimulated M28z T cells (for each pair of bar graphs, M28z, left, MBBz, right). FIG. 6D. Tumor-infiltrating M28z CAR T cells isolated from progressing tumors express inhibitory receptors PD-1, Tim-3, and Lag-3. FIG. 6E. Single-cell tumor suspensions harvested from mice treated with M28z CAR T cells express high levels of PD-1 binding ligands. FIG. 6F. In vitro cultured mesothelioma tumor cells express the ligands (PD-L1, PD-L2) for the PD-1 receptor, and expression is further upregulated following incubation for 24 h with IFN-γ and TNF-α.

FIGS. 7A-7D show that PD-L1 inhibits CAR T-cell effector function. FIG. 7A. 3T3 fibroblasts were transduced to either express mesothelin alone (MSLN+, left) or coexpress MSLN in addition to PD-L1 (MSLN+PD-L1+, right). FIGS. 7B-7D. M28z and MBBz CAR T-cell effector functions were assessed after stimulation with 3T3 MSLN+ or MSLN+PD-L1+ targets. PD-L1 inhibits M28z and MBBz CAR T-cell accumulation upon repeated antigen stimulation (FIG. 7B), cytolytic function following two stimulations with MSLN+PD-L1+ tumor cells (FIG. 7C), and Th1 effector cytokine secretion upon the first stimulation (FIG. 7D). Data represent the mean:SEM of three replicates or are plotted as individual points.

FIGS. 8A-8E show that cotransduction of a PD-1 dominant negative receptor (PD-1 DNR) rescues M28z CAR T cells from PD-1 Ligand-mediated inhibition in vitro and in vivo. FIG. 8A. (Left) Schematic representations of CD28-costimulated T cells binding tumor ligand via the endogenous PD-1 receptor (transmitting a coinhibitory signal) or a cotransduced PD-1 DNR lacking an inhibitory signaling domain. (Right) For in vitro and in vivo experiments, M28z CAR T cells were cotransduced with either empty vector (EV; SFG-mCherry) or PD-1 DNR (SFG-2A-PD-1 DNR). CAR T cells sorted for mCherry expression were then incubated for 24 h with MSLN+ tumor cells that had been treated with IFN-γ and TNF-α to upregulate PD-1 ligands. M28z PD-1 DNR CAR T cells demonstrated a small but statistically significant enhancement in accumulation upon repeated antigen stimulation (FIG. 8B; triangles, M28z EV; squares, M28z PD-1 DNR), an enhanced cytolytic function, as measured by chromium release assay upon the 3rd stimulation with MSLN+PD-L1+ tumor cells (FIG. 8C; triangles, M28z EV; squares, M28z PD-1 DNR), and an increased expression of Th1 supernatant cytokines upon initial stimulation (FIG. 8D). Student's t tests were performed, and statistical significance was determined using the Bonferroni correction for multiple comparisons (*P<0.05; **P<0.01; ***P<0.00I). Data represent the mean±SEM of triplicates or are plotted as individual points. FIG. 8E. Tumor BLI (left) and Kaplan-Meier survival analysis (right) comparing the in vivo efficacy of a single dose of 5e4 M28z EV (n=19) or M28z PD-1 DNR (n=16) pleurally administrated. Data shown are a combination of two independent experiments. The (t) symbol indicates death. Median survival is shown in days. The survival curve was analyzed using the log-rank test (P=0.001). The log-rank test for each independent experiment was significant to the P<0.05 level; two experiments are combined for illustration. A cohort of the mice (M28z PD-1 DNR) in this experiment survived beyond 450 days in spite of repeated tumor rechallenge, demonstrating the “functional persistence” of CAR T cells transduced with PD-1 DNR.

FIG. 9 shows efficient retroviral transduction of human T cells to express Mz, M28z, and MBBz CARs. (Top) Shown is representative FACS analysis 4 days after gene transfer. Fluorescence minus one staining was used to set positive gates after a live/dead stain excluded nonviable cells. All experiments used T cells with 50% to 70% CAR transduction efficiency; transduction percentages between T-cell groups were within 5% of each other. (Bottom) Both CD4+ and CD8+ T-cell subsets were efficiently transduced. CD4+ and CD8+ percentages after gating for CAR T cells are shown.

FIG. 10 shows that MBBz CAR T cells express a less exhausted, more potent phenotype compared to M28z CAR T cells. 4-1BB- and CD28-costimulated T cells were expanded with repeated antigen stimulation, and mRNA was extracted and subjected to RT-PCR analysis 20 h after the third stimulation. Data are represented in fold change relative to the mRNA expression of CD4+ unstransduced T cells. MBBz CAR T cells express higher levels of EOMES (Eomesodermin) and TBX21 (T-bet), and lower levels of PDCD1 (PD-1) and FOXP3 (Foxp3). All comparisons were significant at P<0.001. Results were similar in 3 separate experiments using different donors. Each group of bar graphs shows, left to right, UT (untransduced T cells used as a control), M28z, MBBz.

FIG. 11 shows M28z and MBBz CAR T cells coexpress PD-1 along with other inhibitory receptors. Tumor-infiltrating M28z and MBBz CAR T cells were harvested 6 days following intrapleural administration to pleural tumor bearing mice. Cells were costained with antibodies for PD-1 and for either LAG-3 (left) or TIM-3 (right) and analyzed by flow cytometry. Isotype staining controls (top) were used to establish positive gates.

FIGS. 12A-12E show that cotransduction of PD-1 receptor-targeting shRNAs rescues M28z CAR T cells from PD-L1/PD-1-mediated inhibition in vitro. FIG. 12A. (Left) Schematic representation of CD28-costimulated T cells binding tumor-expressed PD-L1 via endogenous PD-1 receptor, with or without coexpression of PD-1-targeting shRNA. (Right) All experiments included M28z CAR T cells cotransduced with one of two PD-i-targeting shRNAs (sh1 or sh2 coexpressing a dsRED reporter) or with an shRNA targeting a bacterial sequence (KanR). FIG. 12B. Compared with KanR-transduced cells, M28z CAR T cells cotransduced with PD-1-targeting shRNAs demonstrated a 60% to 70% knockdown in PD-1 receptor protein expression upon stimulation with phytohemagglutinin (graphs left to right correspond to 430, 722, 813 and 1411). Cells were incubated with either 3T3 fibroblasts overexpressing PD-L1 (3T3 MSLN+PD-L1+) or mesothelioma tumor cells that had been treated with IFN-γ and TNF-α in order to upregulate PD-L1 and PD-L2. M28z PD1 shRNA CAR T cells demonstrate enhanced accumulation upon repeated antigen stimulation (FIG. 12C), enhanced cytolytic function at low effector to target ratios, as measured by luciferase activity of remaining live tumor cells (FIG. 12D; each group of bar grafts, from left to right, Sh1, Sh2, ShK), and increased Th1 cytokine secretion (FIG. 12E; each group of bar grafts, from left to right, Shi, Sh2, ShK) (**P<0.01; ***P<0.001). Student's ttests were performed and statistical significance was determined using the Bonferroni correction for multiple comparisons. Data represent the mean:SEM of three replicates.

FIGS. 13A-13D show that MBBz CAR T cells prolong tumor-free survival in a mouse model of metastatic lung cancer that includes expression of PD-1 receptor and complementary ligands. FIG. 13A. Single-cell tumor suspensions (A549 lung cancer cells) harvested from mice treated with M28z CAR T cells express high levels of PD-L1. FIG. 13B. In vitro cultured lung cancer cells express the ligands (PD-L1, PD-L2) for PD-1 receptor, and expression is further upregulated following incubation for 24 h with IFN-γ. FIG. 13C. In vivo, BLI was used to monitor tumor burden (firefly luciferase+ MSLN+) in NOD/SCID/γ_{c}^{null} mice. Mice with established lung tumor were treated with 5×10⁴ MBBz or M28z CAR T cells or no T cells. FIG. 13D. Kaplan-Meier survival analysis comparing in vivo efficacy of intrapleurally administered M28z (n=6, middle graph) or MBBz (n=7, right graph) CAR T cells or no T cells (n=3, left graph). The survival curve was analyzed using the log-rank test. **P<0.01.

FIGS. 14A-14C show the results of PD1 DNR transduction into T cells transduced to express the MBBz CAR. From donor 1, human T cells were isolated and transduced with MBBz or MBBzPD1DNR CAR constructs, both with a mcherry marker to identify CAR transduced T cells. FIGS. 14A and 14B show FACS analysis of the transduced cells; FIG. 14A, MBBz mcherry; FIG. 14B, MBBz PD1 mcherry. Staining with PD-1 antibody shows the expression of PD1 DNR in transduced T cells (FIG. 14C). FIG. 14C, A, MBBz mcherry sorted cells; B, MBBz PD1 mcherry sorted cells; C, M28z unstained.

FIGS. 15A-15C show the results of PD1 DNR transduction into T cells transduced to express the M28z CAR. From donor 2, human T cells were isolated and transduced with M28z or M28zPD IDNR CAR constructs, both with a mcherry marker to identify CAR transduced T cells. FIGS. 15A and 15B show FACS analysis of the transduced cells; FIG. 15A, M28z PD1 mcherry; FIG. 15B, M28z mcherry. Staining with PD-1 antibody shows the expression of PD1 DNR in transduced T cells (FIG. 15C). FIG. 15C, A, M28z PD1 DNR mcherry sorted cells; B, M28z mcherry sorted cells.

FIGS. 16A-16D show the efficacy of cells transduced with MBBz versus MBBz PD1 DNR CAR constructs in vitro. In human T cells isolated from donor 1, both MBBz and MBBz PD1DNR transduced cells were exposed to antigen-expressing (mesothelin) targets and analyzed for T-cell accumulation, cytokine secretion and cytotoxicity. FIG. 16A shows accumulation analysis of cells transduced with MBBz PD1 DNR versus MBBz. FIG. 16B shows the percentage of CAR positive cells. FIG. 16B, MBBz mcherry, left bar; MBBzPD1 DNR mcherry, right bar, at day 0 and day 7, respectively. FIG. 16C shows cytokine analysis (IL-2, IFN-γ, TNF-α and GM-CSF) following the first MGM (mesothelin expressing cells) activation. FIG. 16C, MBBz, circles (left in respective graphs), MBBz PD1 DNR, squares (right in respective graphs). FIG. 16D shows lysis of antigen-expressing (mesothelin) cells.

FIGS. 17A-17D show the efficacy of cells transduced with MBBz versus MBBz PD1 DNR CAR constructs in vitro. In human T cells isolated from donor 2, both MBBz and MBBz PD1DNR transduced cells were exposed to antigen-expressing (mesothelin) targets and analyzed for T-cell accumulation, cytokine secretion and cytotoxicity. In this experiment, cytotoxicity was measured repeatedly after repeated antigen exposure. FIG. 17A shows accumulation analysis of MBBz and MBBz PD1 DNR transduced cells. FIG. 17B shows the percentage of CAR transduction. FIG. 17B, MBBz mcherry left bar; MBBz PD1 DNR cherry, right bar. FIG. 17C shows lysis of antigen-expressing (mesothelin) cells after a first and third stimulation with MGM (mesothelin expressing cells). FIG. 17D shows cytokine analysis for IL-2 and IFN-γ in cells transduced with MBBz or MBBz PD-1 DNR. FIG. 17D, MBBz mcherry, left (circles), MBBz mcherry PD-1, right (squares) at each stimulation (STIM), respectively.

FIGS. 18A-18C show the results of PD1 DNR transduction into T cells transduced to express the MBBz CAR. From donor 3, human T cells were isolated and transduced with MBBz or MBBzPD1DNR CAR constructs, both with a mcherry marker to identify CAR transduced T cells. FIGS. 18A and 18B show FACS analysis of cells transduced with MBBz PD1 DNR or MBBz. Staining was performed before and after stimulation. Staining with PD-1 antibody shows the expression of PD1 DNR in transduced T cells (FIG. 18C). FIG. 18C, A, MBBz mcherry sorted cells; B, MBBz mcherry PD1 DNR sorted cells.

FIGS. 19A-19C show the results of PD1 DNR transduction into T cells transduced to express the MBBz CAR. MBBz or MBBzPD1DNR CAR T cell accumulation was tested with or without IL-2 in the media. FIG. 19A shows percentage of CAR transduction at 0, 9, 16 and 23 days following initial stimulation. FIG. 19A, bars left to right: MBBz; MBBz+IL-2 20UI/mL; MBBz+IL-2 40 UI/mL; MBBz+PD1 antibody (Ab) 10 μg/mL; MBBz+PD1 DNR; MBBz PD1 DNR+IL-2 20 UI/mL, on respective days 0, 9, 16 and 23 following initial stimulation. FIG. 19B shows total CAR positive cells at stimulus 1, MGM (mesothelin expressing cells) pretreated. FIG. 19B, bars left to right: MBBz; MBBz+PD1 antibody; MBBz+PD1 DNR; MBBz+PD1 DNR+IL-2 20 IU/mL. FIG. 19C shows the fold increase of MBBz transduced cells versus MBBz transduced cells treated with IL-2, and MBBz PD1 DNR transduced cells versus MBBz PD1 DNR transduced cells treated with IL-2. FIG. 19C, respective sets of bars, left, MBBz versus MBBz+IL2; right MBBz PD1 DNR versus MBBz PD1 DNR IL2.

FIGS. 20A-20D show the efficacy of cells transduced with MBBz and MBBz PD1 DNR CAR constructs in vivo. Mice with established pleurla tumor were treated with a single dose of T cells expressing MBBz or MBBzPD1DNR CAR. Following tumor eradication, mice were rechallenged with either pleural or peritoneal tumor (FIG. 20A), and CAR T-cell functional persistence was assessed by tumor regression and eradication by bioluminescence imaging (BLI). As shown in FIG. 20B-20D, three groups of mice (each group represented in a separate graph) were treated with a single low dose of MBBz, MBBz PD1 DNR or MBBz+PD1 blocking antibody. Each line in the graph indicates one mouse.

FIG. 21 shows analysis of cytokines in mouse serum following re challenge at day 100. The functional persistence of both MBBz and MBBz PD1 DNR transduced CAR T cells was shown by detection of human cytokines (IFN-γ and GM-CSF) in mouse serum. The graphs show from left to right, circle (empty mouse)—mice with no tumor and no treatment; square (control MGM no T cells)—mice with tumor, no treatment; triangle (MBBz mcherry)—mice with tumor treated with MBBz CAR T cells; triangle (MBBz mcherry+PD-1 Ab)—mice with tumor treated with MBBz+PD1 blocking antibody; diamond (MBBz mcherry PD1 DNR)—mice with tumor treated with MBBz PD1 DNR CAR T cells.

FIG. 22 shows effect of expressing a switch receptor in CAR T cells. To rescue the PD-1/PD-L1 mediated inhibition, a PD-1 4-1BB (a switch receptor) construct was cotransduced into T cells expressing M28z CAR, and a third stimulation was induced following PD-L1 engagement. Human T cells were transduced with M28z or M28z PD1 4-1BB CAR, both with a mcherry marker, and were flow sorted and tested for cytokine secretion (IL-2 and IFN-γ) and T-cell accumulation.

FIGS. 23A-23D show the effect of conversion of tumor-mediated PD-L1 inhibition into CAR T cell costimulation to potentiate thoracic cancer. FIG. 23A shows tumor harvest analysis, showing PD-1 and PD-L1 upregulation on CAR T cells and tumor cells. FIG. 23B shows that the addition of PD-1 blocking potentiates CAR T cell therapy in vivo, but its efficacy requires multiple injections. FIG. 23C shows a schematic of a CAR and PD-1 coinhibition versus a CAR coexpressed with a PD-1 DNR (left). FIG. 23C also shows that a single dose of M28z T cells coexpressing PD1-DNR restores effector functions, enhances tumor burden control, and prolongs median survival (two graphs on right). FIG. 23D shows a schematic of a CAR co-expressed with a switch receptor (left). FIG. 23D also shows that converting PD-L1 inhibition into a positive costimulatory signal by a PD-¼-1BB construct cotransduced into M28z CAR T cells enhanced cytokine secretion and T cell accumulation (IL-2, IFN-γ and accumulation shown in graphs left to right).

### 6. DETAILED DESCRIPTION OF THE INVENTION

The present invention relates to compositions and methods for treating cancer. It is known that malignant cells adapt to generate an immunosuppressive microenvironment to protect the cells from immune recognition and elimination. The immunosuppressive microenvironment provides a mechanism for cancer cells and/or tumors to inhibit the effects of a patient's immune system to avoid tumor growth inhibition or elimination. This tumor microenvironment poses a challenge to methods of treatment involving stimulation of an immune response, including immunotherapy methods such as targeted T cell therapies. The present invention is based on the discovery that the effectiveness of cell-based immunotherapy methods can be enhanced by modifying the cells used in immunotherapy to express certain proteins that overcome the immunosuppressive microenvironment. As described herein, immunotherapy cells can be genetically engineered to intrinsically express proteins that are dominant negative mutants and that inhibit blockades that limit the anticancer effect of the immune cells used in immunotherapy. By inhibiting the blockade, immune cells are permitted to provide a more effective immune response against the cancer.

In one aspect, provided herein are cells that are immune cells, or precursor cells thereof, that recombinantly express (a) a chimeric antigen receptor (CAR), and (b) a dominant negative form of an inhibitor of a cell-mediated immune response of the immune cell, wherein the CAR binds to a cancer antigen. Also provided are pharmaceutical compositions comprising a therapeutically effective amount of the cells; and a pharmaceutically acceptable carrier. Additionally provided are polypeptides encoding dominant negative forms of an immune checkpoint inhibitor, for example, containing (a) at least a portion of an extracellular domain of an immune checkpoint inhibitor, said portion comprising the ligand binding region, and (b) a transmembrane domain, wherein the polypeptide is a dominant negative form of the immune checkpoint inhibitor, which polypeptide can optionally be purified. Also provided are T cells that recognize and are sensitized to a cancer antigen, wherein the T cells recombinantly express a dominant negative form of an inhibitor of a T cell-mediated immune response. Further provided are nucleic acids encoding the dominant negative forms of an immune checkpoint inhibitor, as well as vectors encoding the nucleic acids. In another aspect, provided herein are methods of treating a cancer in a subject in need thereof, comprising administering to the subject a therapeutically effective amount of the cells, described above, that recombinantly expresses a CAR and a dominant negative form of an inhibitor of a cell-mediated immune response, wherein the cancer antigen is an antigen of the cancer. Additionally provided are methods of treating a cancer in a subject in need thereof, comprising administering to the subject a pharmaceutical composition comprising a therapeutically effective amount of the cells, described above, and a pharmaceutically acceptable carrier, wherein the cancer antigen is an antigen of the cancer. Further provided are methods of treating a cancer in a subject in need thereof, comprising administering to the subject a therapeutically effective amount of a T cell that recognizes and is sensitized to a cancer antigen, where the T cells recombinantly express a dominant negative form of an inhibitor of a T cell-mediated immune response, wherein the cancer antigen is an antigen of the cancer.

### 6.1 Cells

In one embodiment, the invention provides cells that are immune cells, or precursor cells thereof, that recombinantly express (i) a CAR that binds to a cancer antigen and (ii) a dominant negative form (hereinafter “DN form”) of an inhibitor of a cell-mediated immune response, preferably of the immune cell. The recombinant cells can be used to enhance or provide an immune response against a target such as a cancer antigen. Preferably, the cells are derived from a human (are of human origin prior to being made recombinant) (and human-derived cells are particularly preferred for administration to a human in the methods of treatment of the invention).

The immune cells of the invention can be cells of the lymphoid lineage. Non-limiting examples of cells of the lymphoid lineage that can be used as immune cells include T cells and Natural Killer (NK) cells. T cells express the T cell receptor (TCR), with most cells expressing a and p chains and a smaller population expressing γ and δ chains. T cells useful as immune cells of the invention can be CD4⁺ or CD8⁺ and can include, but are not limited to, T helper cells (CD4⁺), cytotoxic T cells (also referred to as cytotoxic T lymphocytes, CTL; CD8⁺ T cells), and memory T cells, including central memory T cells, stem-cell-like memory T cells (or stem-like memory T cells), and effector memory T cells, for example, T_{EM} cells and T_{EMRA} (CD45RA⁺) cells, natural killer T cells, mucosal associated invariant T cells (MAIT), and γδ T cells. Other exemplary immune cells include, but are not limited to, macrophages, antigen presenting cells (APCs) such as dendritic cells, or any immune cell that expresses an inhibitor of a cell-mediated immune response, for example, an immune checkpoint inhibitor pathway receptor, e.g., PD-1 (in such instance expression of the DN form in the cell inhibits the inhibitor of the the cell-mediated immune response to promote sustained activation of the cell). Precursor cells of immune cells that can be used according to the invention, which recombinantly express a CAR and a DN form, as described above, are, by way of example, hematopoietic stem and/or progenitor cells. Hematopoietic stem and/or progenitor cells can be derived from bone marrow, umbilical cord blood, adult peripheral blood after cytokine mobilization, and the like, by methods known in the art, and then are genetically engineered to recombinantly express a CAR and DN form. Particularly useful precursor cells are those that can differentiate into the lymphoid lineage, for example, hematopoietic stem cells or progenitor cells of the lymphoid lineage.

Immune cells and precursor cells thereof can be isolated by methods well known in the art, including commercially available isolation methods (see, for example, Rowland-Jones et al., Lymphocytes: A Practical Approach, Oxford University Press, New York (1999)). Sources for the immune cells or precursor cells thereof include, but are not limited to, peripheral blood, umbilical cord blood, bone marrow, or other sources of hematopoietic cells. Various techniques can be employed to separate the cells to isolate or enrich for desired immune cells. For instance, negative selection methods can be used to remove cells that are not the desired immune cells. Additionally, positive selection methods can be used to isolate or enrich for desired immune cells or precursor cells thereof, or a combination of positive and negative selection methods can be employed. Monoclonal antibodies (MAbs) are particularly useful for identifying markers associated with particular cell lineages and/or stages of differentiation for both positive and negative selections. If a particular type of cell is to be isolated, for example, a particular type of T cell, various cell surface markers or combinations of markers, including but not limited to, CD3, CD4, CD8, CD34 (for hematopoietic stem and progenitor cells) and the like, can be used to separate the cells, as is well known in the art (see Kearse, T Cell Protocols: Development and Activation, Humana Press, Totowa NJ (2000); De Libero, T Cell Protocols, Vol. 514 of Methods in Molecular Biology, Humana Press, Totowa NJ (2009)).

Procedures for separation of cells include, but are not limited to, density gradient centrifugation, coupling to particles that modify cell density, magnetic separation with antibody-coated magnetic beads, affinity chromatography; cytotoxic agents joined to or used in conjunction with a monoclonal antibody (mAb), including, but not limited to, complement and cytotoxins, and panning with an antibody attached to a solid matrix, for example, a plate or chip, elutriation, flow cytometry, or any other convenient technique (see, for example, Recktenwald et al., Cell Separation Methods and Applications, Marcel Dekker, Inc., New York (1998)).

The immune cells or precursor cells thereof can be autologous or non-autologous to the subject to which they are administered in the methods of treatment of the invention. Autologous cells are isolated from the subject to which the engineered cells recombinantly expressing a CAR and DN form are to be administered. Optionally, the cells can be obtained by leukapheresis, where leukocytes are selectively removed from withdrawn blood, made recombinant, and then retransfused into the donor. Alternatively, allogeneic cells from a non-autologous donor that is not the subject can be used. In the case of a non-autologous donor, the cells are typed and matched for human leukocyte antigen (HLA) to determine an appropriate level of compatibility, as is well known in the art. For both autologous and and non-autologous cells, the cells can optionally be cryopreserved until ready to be used for genetic manipulation and/or administration to a subject using methods well known in the art.

Various methods for isolating immune cells that can be used for recombinant expression of a CAR have been described previously, and can be used, including but not limited to, using peripheral donor lymphocytes (Sadelain et al., Nat. Rev. Cancer 3:35-45 (2003); Morgan et al., Science 314:126-129 (2006), using lymphocyte cultures derived from tumor infiltrating lymphocytes (TILs) in tumor biopsies (Panelli et al., J. Inmunol. 164:495-504 (2000); Panelli et al., J Immunol. 164:4382-4392 (2000)), and using selectively in vitro-expanded antigen-specific peripheral blood leukocytes employing artificial antigen-presenting cells (AAPCs) or dendritic cells (Dupont et al., Cancer Res. 65:5417-5427 (2005); Papanicolaou et al., Blood 102:2498-2505 (2003)). In the case of using stem cells, the cells can be isolated by methods well known in the art (see, for example, Klug et al., Hematopoietic Stem Cell Protocols, Humana Press, New Jersey (2002); Freshney et al., Culture of Human Stem Cells, John Wiley & Sons (2007)).

In a second embodiment, the invention provides T cells that recognize and are sensitized to a cancer antigen, and also which recombinantly express a DN form of an inhibitor of a T cell-mediated immune response. Such T cells can but need not express a CAR that binds to a cancer antigen, since the cells already are cancer antigen-specific so that their immune response (for example, cytotoxicity) is stimulated specifically by such cancer antigen (generally in the form of a cell expressing the cancer antigen on its cell surface). Such T cells that recognize and are sensitized to a cancer antigen can be obtained by known methods, by way of example, in vitro sensitization methods using naive T cells (see, for example, Wolfl et al., Nat. Protocols 9:950-966 (2014)) or hematopoietic progenitor cells (see van Lent et al., J. Immunol. 179:4959-4968 (2007)); or obtained from a subject that has been exposed to and is mounting an immune response against the cancer antigen. Methods for isolating an antigen-specific T cell from a subject are well known in the art. Such methods include, but are not limited to, a cytokine capture system or cytokine secretion assay, which is based on the secretion of cytokines from antigen stimulated T cells that can be used to identify and isolate antigen-specific, and expansion of cells in vitro (see Assenmacher et al., Cytometric Cytokine Secretion Assay, in Analyzing T Cell Responses: How to Analyze Cellular Immune Responses Against Tumor Associated Antigens, Nagorsen et al., eds., Chapter 10, pp. 183-195, Springer, The Netherlands (2005); Haney et al., J. Immunol. Methods 369:33-41 (2011); Bunos et al., Vox Sanguinis DOI: 10.1111/vox.12291 (2015); Montes et al., Clin. Exp. Immunol. 142:292-302 (2005); Adusumilli et al., Sci Transl Med. 6:261ra151 (2014)). Such cytokines include, but are not limited to interferon-γ and tumor necrosis factor-ac. The antigen-specific T cells can be isolated using well known techniques as described above for isolating immune cells, which include, but are not limited to, flow cytometry, magnetic beads, panning on a solid phase, and so forth. Antigen-specific T cell isolation techniques are also commercially available, which can be used or adapted for clinical applications (see, for example, Miltenyi Biotec, Cambridge, MA; Proimmune, Oxford, UK; and the like).

In a specific embodiment, isolated immune cells and precursor cells are genetically engineered ex vivo for recombinant expression of a DN form and a CAR. In a specific embodiment, isolated T cells are genetically engineered ex vivo for recombinant expression of a DN form. The cells can be genetically engineered for recombinant expression by methods well known in the art.

In an embodiment where cancer antigen sensitized T cells that recombinantly express a DN form are used, and wherein such cells are obtained by in vitro sensitization, the sensitization can occur before or after the T cells are genetically engineered to recombinantly express a DN form. In an embodiment where the sensitized T cells are isolated from in vivo sources, it will be self-evident that genetic engineering occurs of the already-sensitized T cells.

The immune cells or precursor cells thereof can be subjected to conditions that favor maintenance or expansion of the immune cells or precursor cells thereof (see Kearse, T Cell Protocols: Development and Activation, Humana Press, Totowa NJ (2000); De Libero, T Cell Protocols, Vol. 514 of Methods in Molecular Biology, Humana Press, Totowa NJ (2009); Parente-Pereira et al., J. Biol. Methods 1(2) e7 (doi 10.14440/jbm.2014.30) (2014); Movassagh et al., Hum. Gene Ther. 11:1189-1200 (2000); Rettig et al., Mol. Ther. 8:29-41 (2003); Agarwal et al., J. Virol. 72:3720-3728 (1998); Pollok et al., Hum. Gene Ther. 10:2221-2236 (1999); Quinn et al., Hum. Gene Ther. 9:1457-1467 (1998); see also commercially available methods such as Dynabeads™ human T cell activator products, Thermo Fisher Scientific, Waltham, MA)). The immune cells or precursor cells thereof, or cancer antigen sensitized T cells, can optionally be expanded prior to or after ex vivo genetic engineering. Expansion of the cells is particularly useful to increase the number of cells for administration to a subject. Such methods for expansion of immune cells are well known in the art (see Kaiser et al., Cancer Gene Therapy 22:72-78 (2015); Wolfl et al., Nat. Protocols 9:950-966 (2014)). Furthermore, the cells can optionally be cryopreserved after isolation and/or genetic engineering, and/or expansion of genetically engineered cells (see Kaiser et al., supra, 2015)). Methods for cyropreserving cells are well known in the art (see, for example, Freshney, Culture of Animal Cells: A Manual of Basic Techniques, 4th ed., Wiley-Liss, New York (2000); Harrison and Rae, General Techniques of Cell Culture, Cambridge University Press (1997)).

With respect to generating cells recombinantly expressing a DN form or a CAR and DN form, one or more nucleic acids encoding the DN form or the CAR and DN form is introduced into the immune cell or precursor cell thereof using a suitable expression vector. The immune cells (for example, T cells) or precursor cells thereof are preferably transduced with one or more nucleic acids encoding a DN form, or a CAR and DN form. In the case of expressing both a CAR and DN form, the CAR and DN form encoding nucleic acids can be on separate vectors or on the same vector, as desired. For example, a polynucleotide encoding a CAR or DN form of the invention can be cloned into a suitable vector, such as a retroviral vector, and introduced into the immune cell using well known molecular biology techniques (see Ausubel et al., Current Protocols in Molecular Biology, John Wiley and Sons, Baltimore, MD (1999)). Any vector suitable for expression in a cell of the invention, particularly a human immune cell or a precursor cell thereof, can be employed. The vectors contain suitable expression elements such as promoters that provide for expression of the encoded nucleic acids in the immune cell. In the case of a retroviral vector, cells can optionally be activated to increase transduction efficiency (see Parente-Pereira et al., J. Biol. Methods 1(2) e7 (doi 10.14440/jbm.2014.30) (2014); Movassagh et al., Hum. Gene Ther. 11:1189-1200 (2000); Rettig et al., Mol. Ther. 8:29-41 (2003); Agarwal et al., J. Virol. 72:3720-3728 (1998); Pollok et al., Hum. Gene Ther. 10:2221-2236 (1998); Quinn et al., Hum. Gene Ther. 9:1457-1467 (1998); see also commercially available methods such as Dynabeads™ human T cell activator products, Thermo Fisher Scientific, Waltham, MA).

In one embodiment, the vector is a retroviral vector, for example, a gamma retroviral or lentiviral vector, which is employed for the introduction of a CAR or DN form into the immune cell or precursor cell thereof. For genetic modification of the cells to express a CAR and/or DN form, a retroviral vector is generally employed for transduction. However, it is understood that any suitable viral vector or non-viral delivery system can be used. Combinations of a retroviral vector and an appropriate packaging line are also suitable, where the capsid proteins will be functional for infecting human cells. Various amphotropic virus-producing cell lines are known, including, but not limited to, PA12 (Miller et al., Mol. Cell. Biol. 5:431-437 (1985)); PA317 (Miller et al., Mol. Cell. Biol. 6:2895-2902(1986)); and CRIP (Danos et al., Proc. Natl. Acad. Sci. USA 85:6460-6464 (1988)). Non-amphotropic particles are suitable too, for example, particles pseudotyped with VSVG, RD114 or GALV envelope and any other known in the art (Relander et al., Mol. Therap. 11:452-459 (2005)). Possible methods of transduction also include direct co-culture of the cells with producer cells (for example, Bregni et al., Blood 80:1418-1422 (1992)), or culturing with viral supernatant alone or concentrated vector stocks with or without appropriate growth factors and polycations (see, for example, Xu et al., Exp. Hemat. 22:223-230 (1994); Hughes, et al. J. Clin. Invest. 89:1817-1824 (1992)).

Generally, the chosen vector exhibits high efficiency of infection and stable integration and expression (see, for example, Cayouette et al., Human Gene Therapy 8:423-430 (1997); Kido et al., Current Eye Research 15:833-844 (1996); Bloomer et al., J Virol. 71:6641-6649 (1997); Naldini et al., Science 272:263 267 (1996); and Miyoshi et al., Proc. Natl. Acad. Sci. U.S.A. 94:10319-10323 (1997)). Other viral vectors that can be used include, for example, adenoviral, lentiviral, and adeno-associated viral vectors, vaccinia virus, a bovine papilloma virus derived vector, or a herpes virus, such as Epstein-Barr Virus (see, for example, Miller, Hum. Gene 7her. 1(1):5-14 (1990); Friedman, Science 244:1275-1281 (1989); Eglitis et al., BioTechniques 6:608-614 (1988); Tolstoshev et al., Current Opin. Biolechnol. 1:55-61 (1990); Sharp, Lancet 337:1277-1278 (1991); Cornetta et al., Prog. NucleicAcidRes. Mol. Biol. 36:311-322 (1989); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)). Retroviral vectors are particularly well developed and have been used in clinical settings (Rosenberg et al., N. Engl. J. Med. 323:370 (1990); Anderson et al., U.S. Pat. No. 5,399,346).

Particularly useful vectors for expressing a CAR and/or DN form of the invention include vectors that have been used in human gene therapy. In one non-limiting embodiment, a vector is a retroviral vector. The use of retroviral vectors for expression in T cells or other immune cells, including engineered CAR T cells, has been described (see Scholler et al., Sci. Transl. Med. 4:132-153 (2012; Parente-Pereira et al., J. Biol. Methods 1(2):e7 (1-9)(2014); Lamers et al., Blood 117(1):72-82 (2011); Reviere et al., Proc. Natl. Acad. Sci. USA 92:6733-6737 (1995)). In one embodiment, the vector is an SGF retroviral vector such as an SGF γ-retroviral vector, which is Moloney murine leukemia-based retroviral vector. SGF vectors have been described previously (see, for example, Wang et al., Gene Therapy 15:1454-1459 (2008)).

The vectors of the invention employ suitable promoters for expression in a particular host cell. The promoter can be an inducible promoter or a constitutive promoter. In a particular embodiment, the promoter of an expression vector provides expression in an immune cell, such as a T cell, or precursor cell thereof. Non-viral vectors can be used as well, so long as the vector contains suitable expression elements for expression in the immune cell or precursor cell thereof. Some vectors, such as retroviral vectors, can integrate into the host genome. If desired, targeted integration can be implemented using technologies such as a nuclease, transcription activator-like effector nucleases (TALENs), Zinc-finger nucleases (ZFNs), and/or clustered regularly interspaced short palindromic repeats (CRISPRs), by homologous recombination, and the like (Gersbach et al., Nucl. Acids Res. 39:7868-7878 (2011); Vasileva, et al. CellDeath Dis. 6:e1831. (Jul. 23 2015); Sontheimer, Hum. Gene Ther. 26(7):413-424 (2015)).

The vectors and constructs can optionally be designed to include a reporter. For example, the vector can be designed to express a reporter protein, which can be useful to identify cells comprising the vector or nucleic acids provided on the vector, such as nucleic acids that have integrated into the host chromosome. In one embodiment, the reporter can be expressed as a bicistronic or multicistronic expression construct with the CAR or DN form. Exemplary reporter proteins include, but are not limited to, fluorescent proteins, such as mCherry, green fluorescent protein (GFP), blue fluorescent protein, for example, EBFP, EBFP2, Azurite, and mKalamal, cyan fluorescent protein, for example, ECFP, Cerulean, and CyPet, and yellow fluorescent protein, for example, YFP, Citrine, Venus, and YPet. In an additional embodiment, a vector construct can comprise a P2A sequence, which provides for optional co-expression of a reporter molecule. P2A is a self-cleaving peptide sequence, which can be used for bicistronic or multicistronic expression of protein sequences (see Szymczak et al., Expert Opin. Biol. Therapy 5(5):627-638 (2005)).

Assays can be used to determine the transduction efficiency of a CAR and/or DN form using routine molecular biology techniques. If a marker has been included in the construct, such as a fluorescent protein, gene transfer efficiency can be monitored by FACS analysis to quantify the fraction of transduced (for example, GFP⁺) immune cells, such as T cells, or precursor cells thereof, and/or by quantitative PCR. Using a well-established cocultivation system (Gade et al., Cancer Res. 65:9080-9088 (2005); Gong et al., Neoplasia 1:123-127 (1999); Latouche et al., Nat. Biotechnol. 18:405-409 (2000)) it can be determined whether fibroblast AAPCs expressing cancer antigen (vs. controls) direct cytokine release from transduced immune cells, such as T cells, expressing a CAR (cell supernatant LUMINEX (Austin TX) assay for IL-2, IL-4, IL-10, IFN-γ, TNF-α, and GM-CSF), T cell proliferation (by carboxyfluorescein succinimidyl ester (CFSE) labeling), and T cell survival (by Annexin V staining). The influence of CD80 and/or 4-1BBL on T cell survival, proliferation, and efficacy can be evaluated. T cells can be exposed to repeated stimulation by cancer antigen positive target cells, and it can be determined whether T cell proliferation and cytokine response remain similar or diminished with repeated stimulation. The cancer antigen CAR constructs can be compared side by side under equivalent assay conditions. Cytotoxicity assays with multiple E:T ratios can be conducted using chromium-release assays.

In addition to providing a nucleic acid encoding a polypeptide that is a DN form or a CAR in a vector for expression in an immune cell or precursor cell thereof, a nucleic acid encoding the polypeptide can also be provided in other types of vectors more suitable for genetic manipulation, such as for expression of various constructs in a bacterial cell such as E. coli. Such vectors can be any of the well known expression vectors, including commercially available expression vectors (see in Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999).

If desired, a nucleic acid encoding a polypeptide for genetic engineering of a cell of the invention, such as a DN form or a CAR, can be codon optimized to increase efficiency of expression in an immune cell or precursor cell thereof Codon optimization can be used to achieve higher levels of expression in a given cell. Factors that are involved in different stages of protein expression include codon adaptability, mRNA structure, and various cis-elements in transcription and translation. Any suitable codon optimization methods or technologies that are known to one skilled in the art can be used to modify the polynucleotides encoding the polypeptides. Such codon optimization methods are well known, including commercially available codon optimization services, for example, OptimumGene™ (GenScript; Piscataway, NJ), Encor optimization (EnCor Biotechnology; Gainseville FL), Blue Heron (Blue Heron Biotech; Bothell, WA), and the like. Optionally, multiple codon optimizations can be performed based on different algorithms, and the optimization results blended to generate a codon optimized nucleic acid encoding a polypeptide.

Further modification can be introduced to the immune cells or precursor cells thereof of the invention. For example, the cells can be modified to address immunological complications and/or targeting by the CAR to healthy tissues that express the same target antigens as the tumor cells. For example, a suicide gene can be introduced into the cells to provide for depletion of the cells when desired. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. Agents are administered to the subject to which the cells containing the suicide genes have been administered, including but not limited to, gancilovir (GCV) for hsv-tk (Greco et al., Frontiers Pharmacol. 6:95 (2015); Barese et al., Mol. Therapy 20:1932-1943 (2012)), AP1903 for iCasp-9 (Di Stasi et al., N. Engl. J Med. 365:1673-1683 (2011), and cetuximab for EGFRt (U.S. Pat. No. 8,802,374), to promote cell death. In one embodiment, administration of a prodrug designed to activate the suicide gene, for example, a prodrug such as AP1903 that can activate iCasp-9, triggers apoptosis in the suicide gene-activated cells. In one embodiment, iCasp9 consists of the sequence of the human FK506-binding protein (FKBP12; GenBank number, AH002818 (AH002818.1, M92422.1, GI:182645; AH002818.2, GI: 1036032368)) with an F36V mutation, connected through a Ser-Gly-Gly-Gly-Ser linker (SEQ ID NO:48) to the gene encoding human caspase 9 (CASP9; GenBank number, NM001229 (NM_001229.4, GI:493798577)), which has had its endogenous caspase activation and recruitment domain deleted. FKBP12-F36V binds with high affinity to an otherwise bioinert small-molecule dimerizing agent, AP1903. In the presence of AP1903, the iCasp9 promolecule dimerizes and activates the intrinsic apoptotic pathway, leading to cell death (Di Stasi et al., N. Engl. J. Med. 365:1673-1683 (2011)). In another embodiment, the suicide gene is an EGFRt polypeptide. The EGFRt polypeptide can provide for cell elimination by administering anti-EGFR monoclonal antibody, for example, cetuximab. The suicide gene can be expressed on a separate vector or, optionally, expressed within the vector encoding a CAR or DN form, and can be a bicistronic or multicistronic construct joined to a CAR or DN form encoding nucleic acid.

### 6.2 Chimeric Antigen Receptors (CARs)

The CAR that is recombinantly expressed by a cell of the invention has an antigen binding domain that binds to a cancer antigen. In specific embodiments, the CAR can be a “first generation,” “second generation” or “third generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J Immunol. 173:2143-2150 (2004); Sadelain et al., Curr. Opin. Immunol. 21(2):215-223 (2009); Hollyman et al., J. Immunolher. 32:169-180 (2009)).

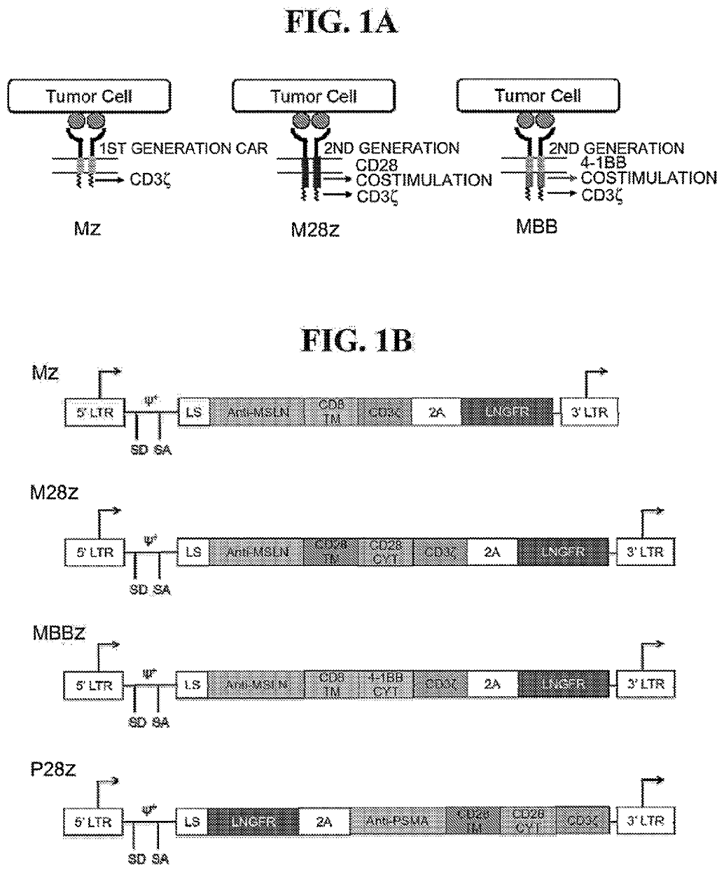

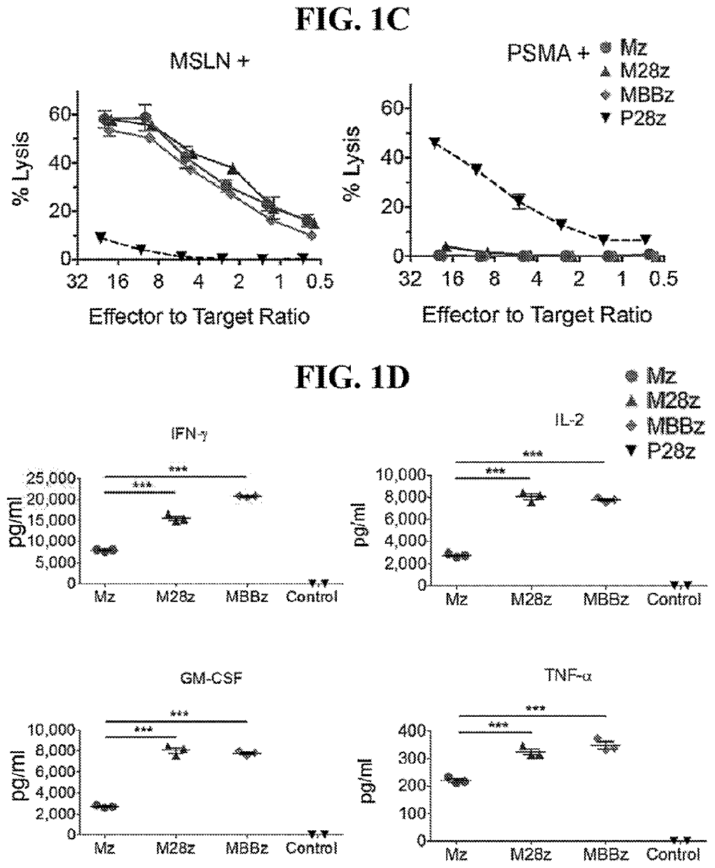

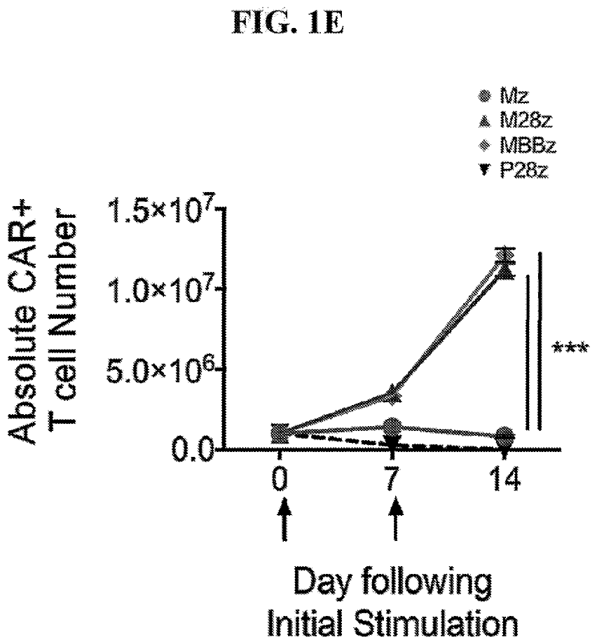

“First generation” CARs are typically composed of an extracellular antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD3ζ-chain, which is the primary transmitter of signals from endogenous T cell receptors (TCRs) (see exemplary first generation CAR in FIG. 1A). “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second-generation” CARs for use in the invention comprise a cancer antigen-binding domain fused to an intracellular signaling domain capable of activating immune cells such as T cells and a co-stimulatory domain designed to augment immune cell, such as T cell, potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell (see exemplary second generation CAR in FIG. 1A). “Second generation” CARs provide both co-stimulation, for example, by CD28 or 4-1BB domains, and activation, for example, by a CD3ζ signaling domain. Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1(9):1577-1583 (2012)). “Third generation” CARs provide multiple co-stimulation, for example, by comprising both CD28 and 4-1BB domains, and activation, for example, by comprising a CD3ζ activation domain.

In the embodiments disclosed herein, the CARs generally comprise an extracellular antigen binding domain, a transmembrane domain and an intracellular domain, as described above, where the extracellular antigen binding domain binds to a cancer antigen. In a particular non-limiting embodiment, the extracellular antigen-binding domain is an scFv.

As disclosed herein, the methods of the invention involve administering cells that have been engineered to co-express a cancer antigen CAR and a dominant negative form (“DN form”) of an inhibitor of a cell-mediated immune response. The extracellular antigen-binding domain of a CAR is usually derived from a monoclonal antibody (mAb) or from receptors or their ligands.

The design of CARs is well known in the art (see, for example, reviews by Sadelain et al., Cancer Discov. 3(4):388-398 (2013); Jensen et al., Immunol. Rev. 257:127-133 (2014); Sharpe et al., Dis. Model Mech. 8(4):337-350 (2015), and references cited therein). A CAR directed to a desired cancer antigen can be generated using well known methods for designing a CAR, including those as described herein. A CAR, whether a first, second or third generation CAR, can be readily designed by fusing a cancer antigen binding activity, for example, an scFv antibody directed to the cancer antigen, to an immune cell signaling domain, such as a T cell receptor cytoplasmic/intracellular domain. As described above, the CAR generally has the structure of a cell surface receptor, with the cancer antigen binding activity, such as an scFv, as at least a portion of the extracellular domain, fused to a transmembrane domain, which is fused to an intracellular domain that has cell signaling activity in an immune cell, such as a T cell, or precursor cell thereof. The cancer antigen CAR can include co-stimulatory molecules, as described herein. One skilled in the art can readily select appropriate transmembrane domains, as described herein and known in the art, and intracellular domains to provide the desired signaling capability in the immune cell, such as a T cell, or precursor cell thereof.

A CAR for use in the present invention comprises an extracellular domain that includes an antigen binding domain that binds to a cancer antigen. The antigen binding domain binds to an antigen on the target cancer cell or tissue. Such an antigen binding domain is generally derived from an antibody. In one embodiment, the antigen binding domain can be an scFv or a Fab, or any suitable antigen binding fragment of an antibody (see Sadelain et al., Cancer Discov. 3:388-398 (2013)). Many antibodies or antigen binding domains derived from antibodies that bind to a cancer antigen are known in the art. Alternatively, such antibodies or antigen binding domains can be produced by routine methods. Methods of generating an antibody are well known in the art, including methods of producing a monoclonal antibody or screening a library to obtain an antigen binding polypeptide, including screening a library of human Fabs (Winter and Harris, Immunol. Today 14:243-246 (1993); Ward et al., Nature 341:544-546 (1989); Harlow and Lane, Antibodies: A Laboratory Manual, Cold Spring Harbor Laboratory Press (1988); Hilyard et al., Protein Engineering: A practical approach (IRL Press 1992); Borrabeck, Antibody Engineering, 2nd ed. (Oxford University Press 1995); Huse et al., Science 246:1275-1281 (1989)). For the CAR, the antigen binding domain derived from an antibody can be human, humanized, chimeric, CDR-grafted, and the like, as desired. For example, if a mouse monoclonal antibody is a source antibody for generating the antigen binding domain of a CAR, such an antibody can be humanized by grafting CDRs of the mouse antibody onto a human framework (see Borrabeck, supra, 1995), which can be beneficial for administering the CAR to a human subject. In a preferred embodiment, the antigen binding domain is an scFv. The generation of scFvs is well known in the art (see, for example, Huston, et al., Proc. Nat. Acad. Sci. USA 85:5879-5883 (1988); Ahmad et al., Clin. Dev. Immunol. 2012: ID980250 (2012); U.S. Pat. Nos. 5,091,513, 5,132,405 and 4,956,778; and U.S. Patent Publication Nos. 20050196754 and 20050196754)).

With respect to obtaining a cancer antigen binding activity, one skilled in the art can readily obtain a suitable cancer antigen binding activity, such as an antibody, using any of the well known methods for generating and screening for an antibody that binds to a desired antigen, as disclosed herein, including the generation of an scFv that binds to a cancer antigen, which is particularly useful in a CAR. In addition, a number cancer antigen antibodies, in particular monoclonal antibodies, are commercially available and can also be used as a source for a cancer antigen binding activity, such as an scFv, to generate a CAR.

Alternatively to using an antigen binding domain derived from an antibody, a CAR extracellular domain can comprise a ligand or extracellular ligand binding domain of a receptor (see Sadelain et al., Cancer Discov. 3:388-398 (2013); Sharpe et al., Dis. Model Mech. 8:337-350 (2015)). In this case, the ligand or extracellular ligand binding domain of a receptor provides to the CAR the ability to target the cell expressing the CAR to the corresponding receptor or ligand. The ligand or extracellular ligand binding domain is selected such that the cell expressing the CAR is targeted to a cancer cell or tumor (see Sadelain et al., Cancer Discov. 3:388-398 (2013); Sharpe et al., Dis. Model Mech. 8:337-350 (2015), and references cited therein). In an embodiment of the invention, the ligand or extracellular ligand binding domain is selected to bind to a cancer antigen that is the corresponding receptor or ligand (see Sadelain et al, Cancer Discov. 3:388-398 (2013)).

For a CAR directed to a cancer antigen, the antigen binding domain of the CAR is selected to bind to an antigen expressed on a cancer cell. Such a cancer antigen can be uniquely expressed on a cancer cell, or the cancer antigen can be overexpressed in a cancer cell relative to noncancerous cells or tissues. The cancer antigen to be bound by the CAR is chosen to provide targeting of the cell expressing the CAR over noncancerous cells or tissues. In one embodiment of the methods of the invention for treating a cancer, an immune cell or precursor cell thereof is designed to treat a cancer patient by expressing in the cell a CAR that binds to a suitable cancer antigen of the patient's cancer, along with a DN form, as described herein.

The cancer antigen can be a tumor antigen. Any suitable cancer antigen can be chosen based on the type of cancer exhibited by a subject (cancer patient) to be treated. It is understood that the selected cancer antigen is expressed in a manner such that the cancer antigen is accessible for binding by the CAR. Generally, the cancer antigen to be targeted by a cell expressing a CAR is expressed on the cell surface of a cancer cell. However, it is understood that any cancer antigen that is accessible for binding to a CAR is suitable for targeting the CAR expressing cell to the cancer cell. Exemplary cancer antigens and exemplary cancers are provided below in Table 1.

TABLE 1
Cancer Antigens and Corresponding Cancer Targets.

| Antigen targeted | Tumors investigated | References¹ |
|---|---|---|
| B7-H3 | Sarcoma and Neuroblastoma | (1) |
| CD276 | | |
| B7-H6 | Ovarian and several solid cancers | (2-4) |
| Nkp30 | | |
| CAIX | Renal cell carcinoma | (5) |
| Carbonic Anhydrase IX | | |
| CEA | Liver metastasis from Colon cancer, Colon, | (6-20) |
| Carcinoembryonic Antigen | Pancreas, Gastric and Lung cancers | |
| CSPG4 | Melanoma, Mesothelioma, Glioblastoma, | (21-24) |
| Chondroitin sulfate proteoglycan-4 | Osteosarcoma, Breast, Head and Neck cancers | |
| DNAM-1 | Melanoma | (25) |
| DNAX Accessory Molecule | | |
| EpHA2 | Glioblastoma and Lung cancer | (26, 27) |
| Ephrin type A Receptor 2 | | |
| EpCAM | Prostate cancer | (28, 29) |
| Epithelial Cell Adhesion Molecule | | |
| ERBB family | Head and Neck and Breast cancers | (30, 31) |
| ERBB2 | Prostate, Breast, Ovarian and Pancreatic cancers, | (32-48) |
| | Glioblastoma, Meduloblastoma, Osteosarcoma, | |
| | Ewing sarcoma, Neuroectodermal tumor, | |
| | Desmoplastic small round cell tumor and | |
| | Fibrosarcoma | |
| EGFRvIII | Glioma/Glioblastoma | (49-56) |
| Epidermal Growth Factor | | |
| Receptor vIII | | |
| FAP | Tumor associated fibroblast in Lung cancer, | (27, 57-59) |
| Fibroblast Associated Protein | Mesothelioma, Breast and Pancreatic cancers | |
| FRα and β | Ovarian cancer | (60-64) |
| Folate Receptor | | |
| GD2 | Neuroblastoma, Edwing sarcoma, Melanoma | (65-71) |
| Disialoganglioside | | |
| GD3 | Melanoma and other Neuroectodermal tumors | (72, 73) |
| Gp100/HLA-A2 | Melanoma | (74, 75) |
| GPC3 | Hepatocellular carcinoma | (76) |
| Glypican 3 | | |
| HERK-V | Melanoma | (77) |
| MAGE-1/HLA-A1 | Melanoma | (78, 79) |
| Melanoma Antigen E | | |
| IL-11Rα | Osteosarcoma | (80) |
| IL-13Rα2 | Glioma/Glioblastoma | (81-87) |
| | Medullobastoma | |
| Lewis-Y | Ovarian | (88) (89, 90) |
| LMP1 | Nasopharyngeal cancer | (91) |
| Latent Membrane Protein 1 | | |
| L1-CAM | Glioblastoma, Neuroblastoma, Ovarian, Lung and | (92, 93) |
| CD271 L1-Cellular Adhesion | Renal carcinoma | |
| Molecule | | |
| Muc-1 | Prostate and Breast cancers | (43, 94-96) |
| Mucin-1 | | |
| Muc-16 | Ovarian cancer | (97, 98) |
| Mucin-16 | | |
| MSLN | Ovarian, Mesothelioma, Lung cancers | (99-107) |
| Mesothelin | | |
| N-cam | Neuroblastoma | (108) |
| CD56 Neural cell-adhesion | | |
| molecule1 | | |
| NKG2DL | Ovarian | (109, 110) |
| NKG2D Ligands | | |
| PSCA | Prostate cancer | (111-113) |
| Prostate Stem cell Antigen | | |
| PSMA | Prostate | (114-117) |
| Prostate Specific Membrane Antigen | | |
| ROR1 | Epithelial solid tumors | (117, 118) |
| Receptor tyrosisne kinase-like | | |
| Orphan Receptor | | |
| TAG72 | Gastrointestinal, Colon and Breast cancers | (119-122) |
| Tumor Associated Glycoprotein 72 | | |
| TRAIL R | Various type of cancer | (123) |
| Trail Receptor | | |
| VEGFR2 | Tumor associated vasculature | (124-127) |
| Vascular Endothelial Growth Factor | | |
| Receptor-2 | | |

¹1. Cheung et al., Hybrid Hybridomics, 22:209-18 (2003); 2. Zhang et al., J Immunol., 189:2290-9 (2012); 3. Wu et al., Gene Ther., 22:675-684 (2015); 4. Wu et al., J Immunol., 194:5305-11 (2015); 5. Lamers et al., Mol Ther., 21:904-12 (2013); 6. Darcy et al., Eur J Immunol., 28:1663-72 (1998); 7. Nolan et al., Clin Cancer Res., 5:3928-41 (1999); 8. Darcy et al., J Immunol., 164:3705-12 (2000); 9. Hombach et al., Gene Ther., 6:300-4 (1999); 10. Haynes et al., J Immunol., 166:182-7 (2001); 11. Haynes et al., J Immunol., 169:5780-6 (2002); 12. Schirrmann et al., Cancer Gene Ther., 9:390-8 (2002); 13. Arakawa et al., Anticancer Res. 2002;22:4285-9. 14. Gyobu et al., Cancer Res., 64:1490-5 (2004); 15. Shibaguchi et al., Anticancer Res., 26:4067-72 (2006); 16. Emtage et al., Clin Cancer Res. 14:8112-22 (2008); 17. Chmielewski et al., Gastroenterology, 143:1095-107 e2 (2012); 18. Chmielewski et al., Gene Ther., 20:177-86 (2013); 19. Burga et al., Cancer Immunol Immunother., 64:817-29 (2015); 20. Katz et al. Clin Cancer Res., 21:3149-59 (2015); 21. Beard et al., J Immunother Cancer, 2:25 (2014); 22. Burns et al., Cancer Res., 70:3027-33 (2010); 23. Geldres et al., Clin Cancer Res., 20:962-71 (2014); 24. Schmidt et al., Proc Natl Acad Sci U S A, 108:2474-9(2011); 25. Wu et al., Cancer Immunol Immunother., 64:409-18 (2015); 26. Chow et al., Mol Ther., 21:629-37 (2013); 27. Kakarla et al., Mol Ther., 21:1611-20 (2013); 28. Shirasu et al., J Biomed Biotechnol., 2012:853879 (2012); 29. Deng et al., BMC Immunol., 16:1(2015); 30. Davies et al., Mol Med., 18:565-76(2012); 31. Papa et al., Methods Mol Biol., 1317:365-82 (2015); 32. Stancovski et al., J Immunol., 151:6577-82 (1993); 33. Moritz et al., Proc Natl Acad Sci U S A, 91:4318-22 (1994); 34. Haynes et al., Cancer Immunol Immunother., 47:278-86 (1999); 35. Pinthus et al., Cancer Res., 63:2470-6 (2003); 36. Ahmed et al., Cancer Res., 67:5957-64 (2007); 37. Li et al., Cancer Gene Ther. 15:382-92 (2008); 38. Wang et al., Clin Cancer Res., 15:943-50 (2009); 39. Ahmed et al., Mol Ther., 17:1779-87 (2009); 40. Zhao et al., J Immunol., 183:5563-74 (2009); 41. Ahmed et al., Clin Cancer Res., 16:474-85 (2010); 42. Duong et al., Immunotherapy, 3:33-48 (2011); 43. Wilkie et al., J Clin Immunol., 32:1059-70 (2012); 44. Lanitis et al., PLoS One, 7 : e49829 (2012); 45. Maliar et al., Gastroenterology, 143:1375-84 el-5 (2012); 46. Rainusso et al., Cancer Gene Ther., 19:212-7 (2012); 47. Sun et al., Breast Cancer Res., 16:R61 (2014); 48. Ahmed et al., J Clin Oncol., 33:1688-96 (2015); 49. Ohno et al., Cancer Sci., 101:2518-24 (2010); 50. Morgan et al., Hum Gene Ther., 23:1043-53 (2012); 51. Choi et al., J Clin Neurosci., 21:189-90 (2014); 52. Ohno et al., J Immunother Cancer, 1:21 (2013); 53. Shen et al., J Hematol Oncol., 6:33 (2013); 54. Sampson et al., Clin Cancer Res., 20:972-84 (2014); 55. Miao et al., PLoS One, 9:e94281 (2014); 56. Johnson et al., Sci Transl Med., 7:275ra22 (2015); 57. Petrausch et al., BMC Cancer, 12:615 (2012); 58. Schuberth et al., J Transl Med., 11:187 (2013); 59. Wang et al., Cancer Immunol Res., 2:154-66 (2014); 60. Parker et al., Hum Gene Ther., 11:2377-87 (2000); 61. Kershaw et al., Clin Cancer Res., 12:6106-15 (2006); 62. Song et al., Cancer Res., 71:4617-27 (2011); 63. Kandalaft et al., J Transl Med., 10:157 (2012); 64. Song et al., Oncotarget, (2015); 65. Krause et al., J Exp Med., 188:619-26 (1998); 66. Rossig et al., Int J Cancer, 94:228-36 (2001); 67. Pule et al., Nat Med., 14:1264-70 (2008); 68. Yvon et al., Clin Cancer Res., 15:5852-60 (2009); 69. Louis et al., Blood, 118:6050-6 (2011); 70. Kailayangiri et al., Br J Cancer, 106:1123-33 (2012); 71. Singh et al., Cancer Immunol Res., 2:1059-70 (2014); 72. Yun et al., Neoplasia, 2:449-59 (2000); 73. Lo et al., Clin Cancer Res., 16:2769-80 (2010); 74. Zhang et al., Immunol Cell Biol., 91:615-24 (2013); 75. Zhang et al., Sci Rep., 4:3571 (2014); 76. Gao et al., Clin Cancer Res., 20:6418-28 (2014); 77. Krishnamurthy et al., Clin Cancer Res., 21:3241-51 (2015); 78. Willemsen et al., Gene Ther., 8:1601-8 (2001); 79. Willemsen et al., J Immunol., 174:7853-8 (2005); 80. Huang et al., Cancer Res., 72:271-81 (2012); 81. Stastny et al., J Pediatr Hematol Oncol., 29:669-77 (2007); 82. Chang et al., Cytotherapy, 9:771-84 (2007); 83. Lazovic et al., Clin Cancer Res., 14:3832-9 (2008); 84. Kong et al., Clin Cancer Res., 18:5949-60 (2012); 85. Hegde et al., Mol Ther., 21:2087-101(2013); 86. Krebs et al., Cytotherapy, 16:1121-31(2014); 87. Brown et al., Clin Cancer Res., (2015); 88. Westwood et al., Proc Natl Acad Sci U S A, 102:19051-6 (2005); 89. Westwood et al., J Immunother., 32:292-301 (2009); 90. Neeson et al., Gene Ther., 17:1105-16 (2010); 91. Tang et al., J Biomed Res., 28:468-75 (2014); 92. Park et al., Mol Ther., 15:825-33 (2007); 93. Hong et al., J Immunother., 37:93-104 (2014); 94. Wilkie et al., J Immunol., 180:4901-9 (2008); 95. Bakhtiari et al., Hybridoma (Larchmt)., 28:85-92 (2009); 96. Sanchez et al., Prostate Cancer Prostatic Dis., 16:123-31, S1 (2013); 97. Chekmasova et al., Discov Med., 9:62-70 (2010); 98. Koneru et al., Oncoimmunology, 4:e994446 (2015); 99. Carpenito et al., Proc Natl Acad Sci U S A, 106:3360-5 (2009); 100. Zhao et al., Cancer Res., 70:9053-61 (2010); 101. Lanitis et al., Mol Ther., 20:633-43 (2012); 102. Riese et al., Cancer Res., 73:3566-77 (2013); 103. Moon et al., Clin Cancer Res., 20:4262-73 (2014); 104. Guedan et al., Blood, 124:1070-80 (2014); 105. Beatty, Oncoimmunology, 3:e28327 (2014); 106. Adusumilli et al., Sci Transl Med., 6:261ra151 (2014); 107. Wang et al., Cancer Immunol Res., 3:815-26 (2015); 108. Gilham et al., J Immunother., 25:139-51 (2002); 109. Barber et al., J Immunol., 183:6939-47 (2009); 110. Song et al., Hum Gene Ther., 24:295-305 (2013); 111. Morgenroth et al., Prostate, 67:1121-31 (2007); 112. Hillerdal et al., BMC Cancer, 14:30 (2014); 113. Abate-Daga et al., Hum Gene Ther., 25:1003-12 (2014); 114. Maher et al., Nat Biotechnol., 20:70-5(2002); 115. Ma et al., Prostate, 61:12-25 (2004); 116. Gade et al., Cancer Res., 65:9080-8 (2005); 117. Hudecek et al., Clin Cancer Res., 19:3153-64 (2013); 118. Deniger et al., PLoS One, 10:e0128151 (2015); 119. Hombach et al., Gastroenterology, 113:1163-70 (1997); 120. McGuinness et al., Hum Gene Ther., 10:165-73 (1999); 121. Patel et al., Cancer Gene Ther. 7:1127-34 (2000); 122. Sharifzadeh et al., Cancer Lett., 334:237-44 (2013); 123. Kobayashi et al., Biochem Biophys Res Commun. 453:798-803 (2014); 124. Chinnasamy et al., Clin Cancer Res., 18:1672-83 (2012); 125. Kanagawa et al., Cancer Gene Ther., 20:57-64 (2013); 126. Chinnasamy et al., Cancer Res., 73:3371-80 (2013); 127. Wang et al., Gene Ther., 20:970-8 (2013); references 128-175, additional references of cancer antigens and corresponding cancer antigen targets; 128. Ordonez, Am J Surg Pathol., 27:1418-28 (2003); 129. Dennis et al., Clin Cancer Res., 11:3766-72 (2005); 130. Alvarez et al., Nanomedicine, 4:295-301 (2008); 131. Rizk et al., Cancer Epidemiol Biomarkers Prev., 21:482-6 (2012); 132. Ordonez, Mod Pathol., 16:192-7 (2003); 133. Frierson et al., Hum Pathol., 34:605-9 (2003); 134. Tchou et al., Breast Cancer Res Treat., 33:799-804 (2012); 135. Parinyanitikul et al., Clin Breast Cancer, 13:378-84 (2013); 136. Wang et al., J Int Med Res., 40:909-16 (2012); 137. Li et al., Breast Cancer Res Treat., 147:675-84 (2014); 138. Ordonez et al., Hum Pathol., 45:1529-40 (2014); 139. Tozbikian et al., PLoS One, 9:e114900 (2014); 140. Bayoglu et al., Biomed Pharmacother., 70:190-5 (2015); 141. Einama et al., Br J Cancer, 107:137-42 (2012); 142. Baba et al., J Surg Oncol., 105:195-9 (2012); 143. Ito et al., Oncol Rep., 31:27-33 (2014); 144. Hassan et al., Am J Clin Pathol., 124:838-45 (2005); 145. Yu et al., J Cancer, 1:141-9 (2010); 146. Kawamata et al., Int J Oncol., 41:2109-18 (2012); 147. Nomura et al., Int Surg., 98:164-9 (2013); 148. Argani Pedram et al., Clin Cancer Res., 7:3862-8 (2001); 149. Swierczynski et al., Hum Pathol. 35:357-66 (2004); 150. Inami et al., Oncol Rep., 20:1375-80 (2008); 151. Frank et al., Am J Clin Pathol., 142:313-9 (2014); 152. Scales et al., Mol Cancer Ther., 13:2630-40 (2014); 153. Liebig et al., Cancer Lett., 223:159-67 (2005); 154. Kawamata et al., J Gastroenterol., 49:81-92 (2014); 155. Miettinen et al., Am J Surg Pathol., 27:150-8 (2003); 156. Ordonez, Am J Surg Pathol., 27:1031-51(2003); 157. Ordonez, Mod Pathol. 19:417-28 (2006); 158. Kushitani et al., Pathol Int., 57:190-9 (2007); 159. Pu et al., Diagn Cytopathol., 36:20-5 (2008); 160. Kachala et al., Clin Cancer Res., 20:1020-8 (2014); 161. Anish et al., Oncotarget, (2015); 162. Pan et al., Hum Pathol., 34:1155-62 (2003); 163. Yuanbin et al., 2014 ASCO Annual Meeting, (2014); 164. Ordonez, Hum Pathol., 35:697-710 (2004); 165. Galloway et al., Histopathology, 48:767-9 (2006); 166. Roe et al., Lung Cancer, 61:235-43 (2008); 167. Tan et al., Hum Pathol., 41:1330-8 (2010); 168. Servais et al., Clin Cancer Res., 18:2478-89 (2012); 169. Drapkin et al., Hum Pathol., 35:1014-21 (2004); 170. Rosen et al., Gynecol Oncol. 99:267-77 (2005); 171. Hassan et al., Appl Immunohistochem Mol Morphol. 13:243-7 (2005); 172. Cao et al., Int J Gynecol Pathol., 24:67-72 (2005); 173. Yen et al., Clin Cancer Res., 12:827-31 (2006); 174. Dainty et al., Gynecol Oncol., 105:563-70 (2007); 175. Obulhasim et al., Eur J Gynaecol Oncol., 31:63-71 (2010).

Suitable antigens include, but are not limited to, mesothelin (MSLN), prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-α and β (FRα and β), Ganglioside G2 (GD2), Ganglioside G3 (GD3), human Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13R(x2), κ-light chain, kinase insert domain receptor (KDR), Lewis A (CA19.9), Lewis Y (LeY), L1 cell adhesion molecule (LiCAM), melanoma-associated antigen 1 (melanoma antigen family Al, MAGE-A1), Mucin 16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLLA-A2, Glypican 3 (GPC3), HA-1H, HERK-V, IL-11Rα, Latent Membrane Protein 1 (LMP1), Neural cell-adhesion molecule (N-CAM/CD56), and Trail Receptor (TRAIL R). It is understood that these or other cancer antigens can be utilized for targeting by a cancer antigen CAR.

In some embodiments of the invention, the CAR is designed to bind to and target cancer cells expressing mesothelin. Mesothelin (MSLN) is an immunogenic cell surface antigen (Ho et al., Clin. Cancer Res. 11:3814-3820 (2005); Hassan et al., Eur. J. Cancer 44:46-53 (2008)) that is highly expressed in solid cancers (Hassan et al., R. & Ho, M. Mesothelin targeted cancer immunotherapy. Eur. J. Cancer 44, 46-53 (2008); Zervos et al., Curr. Opin. Pulm. Med. 14:303-309 (2008); Palumbo et al., Curr. Med. Chem. 15:855-867 (2008); Roe et al., Lung Cancer 61:235-243 (2008); Pass et al., Ann. Thorac. Surg. 85:265-272 (2008); Rodriguez Portal et al., Cancer Epidemiol. Biomarkers Prev. 18(2):646-650 (2009)). MSLN is involved in cell proliferation (Bharadwaj et al., Mol. Cancer Res. 6:1755-1765 (2008)), adhesion (Uehara et al., Mol. Cancer Res. 6:186-193 (2008); Kaneko et al., J. Biol. Chem. 284:3739-3749 (2009)), invasion (Servais et al., C/in. Cancer Res. 18:2478-2489 (2012); Wang et al., J. Int. Med. Res. 40:2109-2116 (2012); Wang et al., J. Int. Med. Res. 40:909-916 (2012)), cell signaling (Uehara et al., N., Mol. Cancer Res. 6:186-193 (2008)), and metastasis (Wu et al., Clin. Cancer Res. 14:1938-1946 (2008)). Studies have demonstrated that serum soluble MSLN-related peptide (SMRP) secreted by MSLN-expressing tumors can be measured in both humans (Pass et al., Ann. Thorac. Surg. 85:265-272 (2008); Cancer Epidemiol. Biomarkers Prev. 18(2):646-650 (2009); Robinson et al., Lung Cancer 49 Suppl 1:S109-S111 (2005); Tajima et al., Anticancer Res. 28:3933-3936 (2008); Park et al., Am. J. Respir. Crit. Care Med. 178:832-837 (2008); Segawa et al., Biochem. Biophys. Res. Commun. 369:915-918 (2008); Amati et al., Cancer Epidemiol. Biomarkers Prev. 17:163-170 (2008); van den Heuvel et al., Lung Cancer 59, 350-354 (2008); Rizk et al., Cancer Epidemiol. Biomarkers Prev. 21:482-486 (2012)) and mice, and has been shown to correlate with therapy response and prognosis. In normal tissues, MSLN is expressed only in the pleura, pericardium, and peritoneum, at low levels (Hassan et al., Eur. J. Cancer 44:46-53 (2008); Bera et al., Mol. Cell. Biol. 20:2902-2906 (2000)). The anti-MSLN recombinant immunotoxin SS1P has shown in vivo specificity and significant antitumor activity in patients (Kelly et al., Mol. Cancer Ther. 11:517-525 (2012); Hassan et al., Clin. Cancer Res. 13:5144-5149 (2007)). In a pancreatic cancer vaccine trial, patients with survival advantage had consistent CD8⁺ T cell responses to MSLN associated with vaccine-induced delayed-type hypersensitivity response (Thomas et al., J Exp. Med. 200:297-306 (2004)). Specific T cell epitopes derived from MSLN were shown to activate human T cells to efficiently lyse human tumors expressing MSLN (Yokokawa et al., Clin. Cancer Res. 11:6342-6351 (2005)).

MSLN-specific CARs have shown efficacy against ovarian cancer, malignant pleural mesothelioma (MPM), and triple-negative breast cancer (TNBC) in both in vitro and in vivo settings (Lanitis et al., Mol. Ther. 20:633-643 (2012); Moon et al., Clin. Cancer Res. 17:4719-4730 (2011); Zhao et al., Cancer Res. 70:9053-9061 (2010); Riese et al., Cancer Res. 73:3566-3577 (2013); Tchou et al., Breast Cancer Res. Treat. 133:799-804 (2012)). Two Phase I clinical trials have used anti-MSLN CAR-transduced T cells. An NCI Phase I clinical trial (ClinicalTrials.gov record NCT01583686) treats metastatic or unresectable cancers that express MSLN with CAR T cells, in combination with myeloablative chemotherapy and/or aldesleukin (an IL-2 analogue) to augment CAR T cell persistence. A University of Pennsylvania Phase I clinical trial (ClinicalTrials.gov record NCTO1355965) gives mesothelioma patients 1 to 3 doses of MSLN-targeted CAR T cells. In the latter study, a human anti-mouse antibody (HAMA) response was observed in the third treated patient (Maus et al., Cancer Immunol. Res. 1(1):26-31 (2013)). In one embodiment, a MSLN-targeted CAR is derived from a human Fab (Feng et al., Mol. Cancer Ther. 8:1113-1118 (2009)), and thus, affords a much decreased risk of immunogenicity, compared with CARs derived from murine antibodies (see Maus et al., Cancer Immunol. Res. 1(1):26-31 (2013)).

In a specific embodiment, one or more nucleic acids encoding a CAR and a DN form are used to transduce both CD4⁺ and CD8⁺ T cells. In such an embodiment, administration of the transduced T cells to a subject should generate both helper and cytotoxic T lymphocyte (CTL) responses in the subject, resulting in a sustained anti-tumor response.

As described above, a CAR also contains a signaling domain that functions in the immune cell, or precursor cell thereof, expressing the CAR. Such a signaling domain can be, for example, derived from CDC or Fc receptor γ (see Sadelain et al., Cancer Discov. 3:388-398 (2013)). In general, the signaling domain will induce persistence, trafficking and/or effector functions in the transduced immune cells such as T cells, or precursor cells thereof (Sharpe et al., Dis. Model Mech. 8:337-350 (2015); Finney et al., J Immunol. 161:2791-2797 (1998); Krause et al., J. Exp. Med. 188:619-626 (1998)). In the case of CDC or Fc receptor γ, the signaling domain corresponds to the intracellular domain of the respective polypeptides, or a fragment of the intracellular domain that is sufficient for signaling. Exemplary signaling domains are described below in more detail.

Exemplary polypeptides are described herein with reference to GenBank numbers, GI numbers and/or SEQ ID NOS. It is understood that one skilled in the art can readily identify homologous sequences by reference to sequence sources, including but not limited to GenBank (ncbi.nlm.nih.gov/genbank/) and EMBL (embl.org/).

CD3ζ. In a non-limiting embodiment, a CAR can comprise a signaling domain derived from a CD3ζ polypeptide, for example, a signaling domain derived from the intracellular domain of CD3ζ, which can activate or stimulate an immune cell, for example, a T cell, or precursor cell thereof. CD3ζ comprises 3 Immune-receptor-Tyrosine-based-Activation-Motifs (ITAMs), and transmits an activation signal to the cell, for example, a cell of the lymphoid lineage such as a T cell, after antigen is bound. A CD3ζ polypeptide can have an amino acid sequence corresponding to the sequence having GenBank No. NP_932170 (NP_932170.1, GI:37595565; see below), or fragments thereof. In one embodiment, the CD3ζ polypeptide has an amino acid sequence of amino acids 52 to 164 of the CD3ζ polypeptide sequence provided below, or a fragment thereof that is sufficient for signaling activity. An exemplary CAR is Mz, which has an intracellular domain comprising a CD3ζ polypeptide comprising amino acids 52 to 164 of the CD3ζ polypeptide sequence provided below. Another exemplary CAR is M28z, which has an intracellular domain comprising a CD3ζ polypeptide comprising amino acids 52 to 164 of the CD3ζ polypeptide provided below. Still another exemplary CAR is MBBz, which has an intracellular domain comprising a CD3ζ polypeptide comprising amino acids 52 to 164 of the CD3ζ polypeptide provided below. Yet another exemplary CAR is P28z, which has an intracellular domain derived from a CD3ζ polypeptide. See GenBank NP_932170 for reference to domains within CD3ζ, for example, signal peptide, amino acids 1 to 21; extracellular domain, amino acids 22 to 30; transmembrane domain, amino acids 31 to 51; intracellular domain, amino acids 52 to 164.

| (NP_932170; SEQ ID NO: 1) | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP QRRKNPQEGL YNELQKDKMA |
| 121 | EAYSEIGMKG ERRRGKGHDG LYQGLSTATK DTYDALHMQA LPPR |

It is understood that a “CD3ζ nucleic acid molecule” refers to a polynucleotide encoding a CD3ζ polypeptide. In one embodiment, the CD3ζ nucleic acid molecule encoding the CD3ζ polypeptide comprised in the intracellular domain of a CAR, including exemplary CARs Mz, M28z, or MBBz, comprises a nucleotide sequence as set forth below.

(SEQ ID NO: 2)
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGCTAA

In certain non-limiting embodiments, an intracellular domain of a CAR can further comprise at least one co-stimulatory signaling domain. Such a co-stimulatory signaling domain can provide increased activation of an immune cell or precursor cell thereof. A co-stimulatory signaling domain can be derived from a CD28 polypeptide, a 4-1BB polypeptide, an OX40 polypeptide, an ICOS polypeptide, a DAP10 polypeptide, a 2B4 polypeptide, and the like. CARs comprising an intracellular domain that comprises a co-stimulatory signaling region comprising 4-1BB, ICOS or DAP-10 have been described previously (see U.S. Pat. No. 7,446,190, which is incorporated herein by reference, which also describes representative sequences for 4-1BB, ICOS and DAP-10). In some embodiments, the intracellular domain of a CAR can comprise a co-stimulatory signaling region that comprises two co-stimulatory molecules, such as CD28 and 4-1BB (see Sadelain et al., Cancer Discov. 3(4):388-398 (2013)), or CD28 and OX40, or other combinations of co-stimulatory ligands, as disclosed herein.

CD28. Cluster of Differentiation 28 (CD28) is a protein expressed on T cells that provides co-stimulatory signals for T cell activation and survival. CD28 is the receptor for CD80 (B7.1) and CD86 (B7.2) proteins. In one embodiment, a CAR can comprise a co-stimulatory signaling domain derived from CD28. For example, as disclosed herein, a CAR can include at least a portion of an intracellular/cytoplasmic domain of CD28, for example an intracellular/cytoplasmic domain that can function as a co-stimulatory signaling domain (see FIG. 1B). A CD28 polypeptide can have an amino acid sequence corresponding to the sequence having GenBank No. P10747 (P10747.1, GI:1 15973) or NP_006130 (NP_006130.1, GI:5453611), as provided below, or fragments thereof. If desired, CD28 sequences additional to the intracellular domain can be included in a CAR of the invention. For example, a CAR can comprise the transmembrane of a CD28 polypeptide. In one embodiment, a CAR can have an amino acid sequence comprising the intracellular domain of CD28 corresponding to amino acids 180 to 220 of CD28, or a fragment thereof. In another embodiment, a CAR can have an amino acid sequence comprising the transmembrane domain of CD28 corresponding to amino acids 153 to 179, or a fragment thereof M28z is an exemplary CAR, which comprises a co-stimulatory signaling domain corresponding to an intracellular domain of CD28 (see FIG. 1B). M28z also comprises a transmembrane domain derived from CD28 (see FIG. 1B). Thus, M28z exemplifies a CAR that comprises two domains from CD28, a co-stimulatory signaling domain and a transmembrane domain. In one embodiment, a CAR has an amino acid sequence comprising the transmembrane domain and the intracellular domain of CD28 and comprises amino acids 153 to 220 of CD28. In another embodiment, a CAR is exemplified by M28z CAR and comprises amino acids 117 to 220 of CD28. Another exemplary CAR having a transmembrane domain and intracellular domain of CD28 is P28z (see FIG. 1B). In one embodiment, a CAR can comprise a transmembrane domain derived from a CD28 polypeptide comprising amino acids 153 to 179 of the CD28 polypeptide provided below. See GenBank NP_006130 for reference to domains within CD28, for example, signal peptide, amino acids 1 to 18; extracellular domain, amino acids 19 to 152; transmembrane domain, amino acids 153 to 179; intracellular domain, amino acids 180 to 220. It is understood that sequences of CD28 that are shorter or longer than a specific delineated domain can be included in a CAR, if desired.

| (NP_006130; SEQ ID NO: 3) | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

It is understood that a “CD28 nucleic acid molecule” refers to a polynucleotide encoding a CD28 polypeptide. In one embodiment, the CD28 nucleic acid molecule encoding the CD28 polypeptide of M28z comprising the transmembrane domain and the intracellular domain, for example, the co-stimulatory signaling region, comprises a nucleotide sequence as set forth below.

(SEQ ID NO: 4)
ATTGAAGTTATGTATCCTCCTCCTTACCTAGACAATGAGAAGAGCAATGG
AACCATTATCCATGTGAAAGGGAAACACCTTTGTCCAAGTCCCCTATTTC
CCGGACCTTCTAAGCCCTTTTGGGTGCTGGTGGTGGTTGGTGGAGTCCTG
GCTTGCTATAGCTTGCTAGTAACAGTGGCCTTTATTATTTTCTGGGTGAG
GAGTAAGAGGAGCAGGCTCCTGCACAGTGACTACATGAACATGACTCCCC
GCCGCCCCGGGCCCACCCGCAAGCATTACCAGCCCTATGCCCCACCACGC
GACTTCGCAGCCTATCGCTCC

4-1BB. 4-1BB, also referred to as tumor necrosis factor receptor superfamily member 9, can act as a tumor necrosis factor (TNF) ligand and have stimulatory activity. In one embodiment, a CAR can comprise a co-stimulatory signaling domain derived from 4-1BB. A 4-1BB polypeptide can have an amino acid sequence corresponding to the sequence having GenBank No. P41273 (P41273.1, GI:728739) or NP_001552 (NP_001552.2, GI:5730095) or fragments thereof. In one embodiment, a CAR can have a co-stimulatory domain comprising the intracellular domain of 4-1BB corresponding to amino acids 214 to 255, or a fragment thereof In another embodiment, a CAR can have a transmembrane domain of 4-1BB corresponding to amino acids 187 to 213, or a fragment thereof. An exemplary CAR is MBBz, which has an intracellular domain comprising a 4-1BB polypeptide (for example, amino acids 214 to 255 of NP_001552, SEQ ID NO:5) (see FIG. 1B). See GenBank NP_001552 for reference to domains within 4-1BB, for example, signal peptide, amino acids 1 to 17; extracellular domain, amino acids 18 to 186; transmembrane domain, amino acids 187 to 213; intracellular domain, amino acids 214 to 255. It is understood that sequences of 4-1BB that are shorter or longer than a specific delineated domain can be included in a CAR, if desired. It is also understood that a “4-1BB nucleic acid molecule” refers to a polynucleotide encoding a 4-1BB polypeptide.

| (NP_001552; SEQ ID NO: 5) | |
|---|---|
| 1 | MGNSCYNIVA TLLLVLNFER TRSLQDPCSN CPAGTFCDNN RNQICSPCPP NSFSSAGGQR |
| 61 | TCDICRQCKG VFRTRKECSS TSNAECDCTP GFHCLGAGCS MCEQDCKQGQ ELTKKGCKDC |
| 121 | CFGTFNDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

OX40. OX40, also referred to as tumor necrosis factor receptor superfamily member 4 precursor or CD134, is a member of the TNFR-superfamily of receptors. In one embodiment, a CAR can comprise a co-stimulatory signaling domain derived from OX40. An OX40 polypeptide can have an amino acid sequence corresponding to the sequence having GenBank No. P43489 (P43489.1, GI:1171933) or NP_003318 (NP_003318.1, GI:4507579), provided below, or fragments thereof. In one embodiment, a CAR can have a co-stimulatory domain comprising the intracellular domain of OX40 corresponding to amino acids 236 to 277, or a fragment thereof In another embodiment, a CAR can have an amino acid sequence comprising the transmembrane domain of OX40 corresponding to amino acids 215 to 235 of OX40, or a fragment thereof. See GenBank NP_003318 for reference to domains within OX40, for example, signal peptide, amino acids 1 to 28; extracellular domain, amino acids 29 to 214; transmembrane domain, amino acids 215 to 235; intracellular domain, amino acids 236 to 277. It is understood that sequences of OX40 that are shorter or longer than a specific delineated domain can be included in a CAR, if desired. It is also understood that an “OX40 nucleic acid molecule” refers to a polynucleotide encoding an OX40 polypeptide.

| (NP_003318; SEQ ID NO: 6) | |
|---|---|
| 1 | MCVGARRLGR GPCAALLLLG LGLSTVTGLH CVGDTYPSND RCCHECRPGN GMVSRCSRSQ |
| 61 | NTVCRPCGPG FYNDVVSSKP CKPCTWCNLR SGSERKQLCT ATQDTVCRCR AGTQPLDSYK |
| 121 | PGVDCAPCPP GHFSPGDNQA CKPWTNCTLA GKHTLQPASN SSDAICEDRD PPATQPQETQ |
| 181 | GPPARPITVQ PTEAWPRTSQ GPSTRPVEVP GGRAVAAILG LGLVLGLLGP LAILLALYLL |
| 241 | RRDQRLPPDA HKPPGGGSFR TPIQEEQADA HSTLAKI |

ICOS. Inducible T-cell costimulator precursor (ICOS), also referred to as CD278, is a CD28-superfamily costimulatory molecule that is expressed on activated T cells. In one embodiment, a CAR can comprise a co-stimulatory signaling domain derived from ICOS. An ICOS polypeptide can have an amino acid sequence corresponding to the sequence having GenBank No. NP_036224 (NP_036224.1, GI:15029518), provided below, or fragments thereof. In one embodiment, a CAR can have a co-stimulatory domain comprising the intracellular domain of ICOS corresponding to amino acids 162 to 199 of ICOS. In another embodiment, a CAR can have an amino acid sequence comprising the transmembrane domain of ICOS corresponding to amino acids 141 to 161 of ICOS, or a fragment thereof. See GenBank NP_036224 for reference to domains within ICOS, for example, signal peptide, amino acids 1 to 20; extracellular domain, amino acids 21 to 140; transmembrane domain, amino acids 141 to 161; intracellular domain, amino acids 162 to 199. It is understood that sequences of ICOS that are shorter or longer than a specific delineated domain can be included in a CAR, if desired. It is also understood that an “ICOS nucleic acid molecule” refers to a polynucleotide encoding an ICOS polypeptide.

| (NP_036224; SEQ ID NO: 7) | |
|---|---|
| 1 | MKSGLWYFFL FCLRIKVLTG EINGSANYEM FIFHNGGVQI LCKYPDIVQQ FKMQLLKGGQ |
| 61 | ILCDLIKTKG SGNTVSIKSL KFCHSQLSNN SVSFFLYNLD HSHANYYFCN LSIFDPPPFK |
| 121 | VTLTGGYLHI YESQLCCQLK FWLPIGCAAF VVVCILGCIL ICWLTKKKYS SSVHDPNGEY |
| 181 | MFMRAVNTAK KSRLTDVTL |

DAP10. DAP10, also referred to as hematopoietic cell signal transducer, is a signaling subunit that associates with a large family of receptors in hematopoietic cells. In one embodiment, a CAR can comprise a co-stimulatory domain derived from DAP10. A DAP10 polypeptide can have an amino acid sequence corresponding to the sequence having GenBank No. NP_055081.1 (GI:15826850), provided below, or fragments thereof. In one embodiment, a CAR can have a co-stimulatory domain comprising the intracellular domain of DAP10 corresponding to amino acids 70 to 93, or a fragment thereof In another embodiment, a CAR can have a transmembrane domain of DAP10 corresponding to amino acids 49 to 69, or a fragment thereof See GenBank NP_055081.1 for reference to domains within DAP10, for example, signal peptide, amino acids 1 to 19; extracellular domain, amino acids 20 to 48; transmembrane domain, amino acids 49 to 69; intracellular domain, amino acids 70 to 93. It is understood that sequences of DAP10 that are shorter or longer than a specific delineated domain can be included in a CAR, if desired. It is also understood that a “DAP10 nucleic acid molecule” refers to a polynucleotide encoding an DAP10 polypeptide.

| (NP_055081.1; SEQ ID NO: 8) | |
|---|---|
| 1 | MIHLGHILFL LLLPVAAAQT TPGERSSLPA FYPGTSGSCS GCGSLSLPLL AGLVAADAVA |
| 61 | SLLIVGAVFL CARPRRSPAQ EDGKVYINMP GRG |

The extracellular domain of a CAR can be fused to a leader or a signal peptide that directs the nascent protein into the endoplasmic reticulum and subsequent translocation to the cell surface. It is understood that, once a polypeptide containing a signal peptide is expressed at the cell surface, the signal peptide has generally been proteolytically removed during processing of the polypeptide in the endoplasmic reticulum and translocation to the cell surface. Thus, a polypeptide such as a CAR is generally expressed at the cell surface as a mature protein lacking the signal peptide, whereas the precursor form of the polypeptide includes the signal peptide. A signal peptide or leader can be essential if a CAR is to be glycosylated and/or anchored in the cell membrane. The signal sequence or leader is a peptide sequence generally present at the N-terminus of newly synthesized proteins that directs their entry into the secretory pathway. The signal peptide is covalently joined to the N-terminus of the extracellular antigen-binding domain of a CAR as a fusion protein. In one embodiment, the signal peptide comprises a CD8 polypeptide comprising amino acids MALPVTALLLPLALLLHAARP (SEQ ID NO:9). It is understood that use of a CD8 signal peptide is exemplary. Any suitable signal peptide, as are well known in the art, can be applied to a CAR to provide cell surface expression in an immune cell (see Gierasch Biochem. 28:923-930 (1989); von Heijne, J. Mol. Biol. 184 (1):99-105 (1985)). Particularly useful signal peptides can be derived from cell surface proteins naturally expressed in the immune cell or precursor cell thereof, including any of the signal peptides of the polypeptides disclosed herein. Thus, any suitable signal peptide can be utilized to direct a CAR to be expressed at the cell surface of an immune cell or precursor cell thereof.

In certain non-limiting embodiments, an extracellular antigen-binding domain of a CAR can comprise a linker sequence or peptide linker connecting the heavy chain variable region and light chain variable region of the extracellular antigen-binding domain. In one non-limiting example, the linker comprises amino acids having the sequence set forth in GGGGSGGGGSGGGGS (SEQ ID NO: 10).

In certain non-limiting embodiments, a CAR can also comprise a spacer region or sequence that links the domains of the CAR to each other. For example, a spacer can be included between a signal peptide and an antigen binding domain, between the antigen binding domain and the transmembrane domain, between the transmembrane domain and the intracellular domain, and/or between domains within the intracellular domain, for example, between a stimulatory domain and a co-stimulatory domain. The spacer region can be flexible enough to allow interactions of various domains with other polypeptides, for example, to allow the antigen binding domain to have flexibility in orientation in order to facilitate antigen recognition. The spacer region can be, for example, the hinge region from an IgG, the CH₂CH₃ (constant) region of an immunoglobulin, and/or portions of CD3 (cluster of differentiation 3) or some other sequence suitable as a spacer.

The transmembrane domain of a CAR generally comprises a hydrophobic alpha helix that spans at least a portion of the membrane. Different transmembrane domains result in different receptor stability. After antigen recognition, receptors cluster and a signal is transmitted to the cell. In an embodiment, the transmembrane domain of a CAR can be derived from another polypeptide that is naturally expressed in the immune cell or precursor cell thereof. In one embodiment, a CAR can have a transmembrane domain derived from CD8, CD28, CD3ζ, CD4, 4-1BB, OX40, ICOS, CTLA-4, PD-1, LAG-3, 2B4, BTLA, or other polypeptides expressed in the immune cell, or precursor cell thereof, having a transmembrane domain, including others as disclosed herein. Optionally, the transmembrane domain can be derived from a polypeptide that is not naturally expressed in the immune cell or precursor cell thereof, so long as the transmembrane domain can function in transducing signal from antigen bound to the CAR to the intracellular signaling and/or co-stimulatory domains. It is understood that the portion of the polypeptide that comprises a transmembrane domain of the polypeptide can include additional sequences from the polypeptide, for example, additional sequences adjacent on the N-terminal or C-terminal end of the transmembrane domain, or other regions of the polypeptide, as desired.

CD8. Cluster of differentiation 8 (CD8) is a transmembrane glycoprotein that serves as a co-receptor for the T cell receptor (TCR). CD8 binds to a major histocompatibility complex (MHC) molecule and is specific for the class I MHC protein. In one embodiment, a CAR can comprise a transmembrane domain derived from CD8. A CD8 polypeptide can have an amino acid sequence corresponding to the sequence having GenBank No. NP_001139345.1 (GI:225007536), as provided below, or fragments thereof. In one embodiment, a CAR can have an amino acid sequence comprising the transmembrane domain of CD8 corresponding to amino acids 183 to 203, or fragments thereof. In one embodiment, an exemplary CAR is Mz, which has a transmembrane domain derived from a CD8 polypeptide (see FIG. 1B). In another embodiment, an exemplary CAR is MBBz, which has a transmembrane domain derived from a CD8 polypeptide (see FIG. 1B). In one non-limiting embodiment, a CAR can comprise a transmembrane domain derived from a CD8 polypeptide comprising amino acids 183 to 203. In addition, a CAR can comprise a hinge domain comprising amino acids 137-182 of the CD8 polypeptide provided below. In another embodiment, a CAR can comprise amino acids 137-203 of the CD8 polypeptide provided below. In yet another embodiment, a CAR can comprise amino acids 137 to 209 of the CD8 polypeptide provided below. See GenBank NP_001139345.1 for reference to domains within CD8, for example, signal peptide, amino acids 1 to 21; extracellular domain, amino acids 22 to 182; transmembrane domain amino acids, 183 to 203; intracellular domain, amino acids 204 to 235. It is understood that additional sequence of CD8 beyond the transmembrane domain of amino acids 183 to 203 can be included in a CAR, if desired. It is further understood that sequences of CD8 that are shorter or longer than a specific dilineated domain can be included in a CAR, if desired. It also is understood that a “CD8 nucleic acid molecule” refers to a polynucleotide encoding a CD8 polypeptide.

| (NP_001139345.1; SEQ ID NO: 11) | |
|---|---|
| 1 | MALPVTALLL PLALLLHAAR PSQFRVSPLD RTWNLGETVE LKCQVLLSNP TSGCSWLFQP |
| 61 | RGAAASPTFL LYLSQNKPKA AEGLDTQRFS GKRLGDTFVL TLSDFRRENE GYYFCSALSN |
| 121 | SIMYFSHFVP VFLPAKPTTT PAPRPPTPAP TIASQPLSLR PEACRPAAGG AVHTRGLDFA |
| 181 | CDIYIWAPLA GTCGVLLLSL VITLYCNHRN RRRVCKCPRP VVKSGDKPSL SARYV |

CD4. Cluster of differentiation 4 (CD4), also referred to as T-cell surface glycoprotein CD4, is a glycoprotein found on the surface of immune cells such as T helper cells, monocytes, macrophages, and dendritic cells. In one embodiment, a CAR can comprise a transmembrane domain derived from CD4. CD4 exists in various isoforms. It is understood that any isoform can be selected to achieve a desired function. Exemplary isoforms include isoform 1 (NP_000607.1, GI:10835167), isoform 2 (NP_001181943.1, GI:303522479), isoform 3 (NP_001181944.1, GI:303522485; or NP_001181945.1, GI:303522491; or NP_001181946.1, GI:303522569), and the like. One exemplary isoform sequence, isoform 1, is provided below. In one embodiment, a CAR can have an amino acid sequence comprising the transmembrane domain of CD4 corresponding to amino acids 397 to 418, or fragments thereof. See GenBank NP_000607.1 for reference to domains within CD4, for example, signal peptide, amino acids 1 to 25; extracellular domain, amino acids 26 to 396; transmembrane domain amino acids, 397 to 418; intracellular domain, amino acids 419 to 458. It is understood that additional sequence of CD4 beyond the transmembrane domain of amino acids 397 to 418 can be included in a CAR, if desired. It is further understood that sequences of CD4 that are shorter or longer than a specific dilineated domain can be included in a CAR, if desired. It also is understood that a “CD4 nucleic acid molecule” refers to a polynucleotide encoding a CD4 polypeptide.

| (NP_000607.1; SEQ ID NO: 12) | |
|---|---|
| 1 | MNRGVPFRHL LLVLQLALLP AATQGKKVVL GKKGDTVELT CTASQKKSIQ FHWKNSNQIK |
| 61 | ILGNQGSFLT KGPSKLNDRA DSRRSLWDQG NFPLIIKNLK IEDSDTYICE VEDQKEEVQL |
| 121 | LVFGLTANSD THLLQGQSLT LTLESPPGSS PSVQCRSPRG KNIQGGKTLS VSQLELQDSG |
| 181 | TWTCTVLQNQ KKVEFKIDIV VLAFQKASSI VYKKEGEQVE FSFPLAFTVE KLTGSGELWW |
| 241 | QAERASSSKS WITFDLKNKE VSVKRVTQDP KLQMGKKLPL HLTLPQALPQ YAGSGNLTLA |
| 301 | LEAKTGKLHQ EVNLVVMRAT QLQKNLTCEV WGPTSPKLML SLKLENKEAK VSKREKAVWV |
| 361 | LNPEAGMWQC LLSDSGQVLL ESNIKVLPTW STPVQPMALI VLGGVAGLLL FIGLGIFFCV |
| 421 | RCRHRRRQAE RMSQIKRLLS EKKTCQCPHR FQKTCSPI |

As disclosed herein, mesothelin CARs exemplify CARs that can target a cancer antigen, and CARs directed to other cancer antigens can be generated using similar methods and others well known in the art, as described above. It is understood that domains of the polypeptides described herein can be used in a cancer antigen CAR, as useful to provide a desired function such as a signal peptide, antigen binding domain, transmembrane domain, intracellular signaling domain and/or co-stimulatory domain. For example, a domain can be selected such as a signal peptide, a transmembrane domain, an intracellular signaling domain, or other domain, as desired, to provide a particular function to a CAR of the invention. Possible desirable functions can include, but are not limited to, providing a signal peptide and/or transmembrane domain.

In one embodiment, the invention provides CARs directed to mesothelin. In certain non-limiting embodiments, MSLN is human mesothelin having the sequence with an NCBI Reference No: AAV87530.1 (GI:56406362), or fragments thereof, as provided below:

(GenBank AAV87530.1; SEQ ID NO: 13)
MALPTARPLL GSCGTPALGS LLFLLFSLGW VQPSRTLAGE
TGQEAAPLDG VLANPPNISS LSPRQLLGFP CAEVSGLSTE
RVRELAVALA QKNVKLSTEQ LRCLAHRLSE PPEDLDALPL
DLLLFLNPDA FSGPQACTHF FSRITKANVD LLPRGAPERQ
RLLPAALACW GVRGSLLSEA DVRALGGLAC DLPGRFVAES
AEVLLPRLVS CPGPLDQDQQ EAARAALQGG GPPYGPPSTW
SVSTMDALRG LLPVLGQPII RSIPQGIVAA WRQRSSRDPS
WRQPERTILR PRFRREVEKT ACPSGKKARE IDESLIFYKK
WELEACVDAA LLATQMDRVN AIPFTYEQLD VLKHKLDELY
PQGYPESVIQ HLGYLFLKMS PEDIRKWNVT SLETLKALLE
VNKGHEMSPQ VATLIDRFVK GRGQLDKDTL DTLTAFYPGY
LCSLSPEELS SVPPSSIWAV RPQDLDTCDP RQLDVLYPKA
RLAFQNMNGS EYFVKIQSFL GGAPTEDLKA LSQQNVSMDL
ATFMKLRTDA VLPLTVAEVQ KLLGPHVEGL KAEERHRPVR
DWILRQRQDD LDTLGLGLQG GIPNGYLVLD LSVQEALSGT
PCLLGPGPVL TVLALLLAST LA

In certain embodiments, the extracellular antigen-binding domain of the anti-mesothelin CAR comprises a human anti-mesothelin antibody or an antigen-binding portion thereof described in U.S. Pat. No. 8,357,783, which is herein incorporated by reference in its entirety. In some embodiments, the extracellular antigen-binding domain is derived from a heavy chain variable region and a light chain variable region of an antibody that binds to human mesothelin, for example, antibody m912 as disclosed in Feng et al., Mol. Cancer Therapy 8(5):1113-1118 (2009), which is herein incorporated by reference in its entirety. Antibody m912 was isolated from a human Fab library by panning against recombinant mesothelin. In other embodiments, the extracellular antigen-binding domain is derived from an Fab, for example, from human or mouse Fab libraries.

In certain embodiments, the extracellular antigen-binding domain or an MSLN CAR comprises a heavy chain variable region comprising amino acids having the sequence set forth below.

(SEQ ID NO: 14)
QVQLQESGPGLVKPSETLSLTCTVSGGSVSSGSYYWSWIRQPPGKGLE
WIGYIYYSGSTNYNPSLKSRVTISVDTSKNQFSLKLSSVTAADTAVYY
CAREGKNGAFDIWGQGTMVTVSSASTKGPSVFPLAPSSKSTSGGTAAL
GCLVKDYFPEPVTVSWNSGALTSGVHTFPAVLQSSGLYSLSSVVTVPS
SSLGTQTYICNVNHKPSNTKVDKKVEPKSCDKTSGQAG

The nucleic acid sequence encoding the amino acid sequence above is set forth below.

| (SEQ ID NO: 15) | |
|---|---|
| caggtgcagctgcaggagtccggcccaggactggtgaagccttcggagaccctgtccctc | 60 |
| acctgcactgtctctggtggctccgtcagcagtggtagttactactggagctggatccgg | 120 |
| cagcccccagggaagggactggagtggattgggtatatctattacagtgggagcaccaac | 180 |
| tacaacccctccctcaagagtcgagtcaccatatcagtagacacgtccaagaaccagttc | 240 |
| tccctgaagctgagctctgtgaccgctgcggacacggccgtgtattactgtgcgagagag | 300 |
| gggaagaatggggcttttgatatctggggccaagggacaatggtcaccgtctcttcagcc | 360 |
| tccaccaagggcccatcggtcttccccctggcaccctcctccaagagcacctctgggggc | 420 |
| acagcggccctgggctgcctggtcaaggactacttccccgaaccggtgacggtgtcgtgg | 480 |
| aactcaggcgccctgaccagcggcgtgcacaccttcccggctgtcctacagtcctcagga | 540 |
| ctctactccctcagcagcgtggtgaccgtgccctccagcagcttgggcacccagacctac | 600 |
| atctgcaacgtgaatcacaagcccagcaacaccaaggtggacaagaaagttgagcccaaa | 660 |
| tcttgtgacaaaactagtggccaggccggccac | 693 |

In some embodiments, the extracellular antigen-binding domain comprises a light chain variable region comprising amino acids having the sequence set forth below.

(SEQ ID NO: 16)
DIQMTQSPSSLSASVGDRVTITCRASQSISSYLNWYQQKPGKAPKLLI
YAASSLQSGVPSGFSGSGSGTDFTLTISSLQPEDFATYYCQQSYSTPL
TEGGGTKVEIKRTVAAPSVFIFPPSDEQLKSGTASVVCLLNNEYPREA
KVQWKVDNALQSGNSQESVTEQDSKDSTYSLSSTLTLSKADYEKHKVY
ACEVTHQGLSSPVTKSFNRGEC

The nucleic acid sequence encoding the amino acid sequence above is set forth below.

| (SEQ ID NO: 17) | |
|---|---|
| gacatccagatgacccagtctccatcctccctgtctgcatctgtaggagacagagtcacc | 60 |
| atcacttgccgggcaagtcagagcattagcagctatttaaattggtatcagcagaaacca | 120 |
| gggaaagcccctaagctcctgatctatgctgcatccagtttgcaaagtggggtcccatca | 180 |
| gggttcagtggcagtggatctgggacagatttcactctcaccatcagcagtctgcaacct | 240 |
| gaagattttgcaacttactactgtcaacagagttacagtaccccgctcactttcggcgga | 300 |
| gggaccaaggtggagatcaaacgaactgtggctgcaccatctgtcttcatcttcccgcca | 360 |
| tctgatgagcagttgaaatctggaactgcctctgttgtgtgcctgctgaataacttctat | 420 |
| cccagagaggccaaagtacagtggaaggtggataacgccctccaatcgggtaactcccag | 480 |
| gagagtgtcacagagcaggacagcaaggacagcacctactgcctcagcagcaccctgacg | 540 |
| ctgagcaaagcagactacgagaaacacaaactctacgcctgcgaagtcacccatcagggc | 600 |
| ctgagctcgcccgtcacaaagagcttcaacaggggagagt | |

In some embodiments, the extracellular antigen-binding domain comprises a light chain variable region comprising amino acids having the sequence set forth below.

(SEQ ID NO: 18)
RHQMTQSPSSLSASVGDRVTITCRASQSISSYLNWYQQKPGKAPKLLIYA
ASSLQSGVPSGFSGSGSGTDFTLTISSLQPEDFATYYCQQSYSTPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

In certain embodiments, the extracellular antigen-binding domain of an MSLN CAR comprises a single-chain variable fragment (scFv). In one specific embodiment, the extracellular antigen-binding domain of a CAR comprises a human scFV. In one embodiment, the human scFV comprises a heavy chain variable region comprising amino acids 1-119 of the MSLN CAR described above (SEQ ID NO: 14). In another embodiment, the human scFV of an MSLN CAR comprises a heavy chain variable region comprising amino acids having the sequence set forth below.

(SEQ ID NO: 19)
QVQLQESGPGLVKPSETLSLTCTVSGGSVSSGSYYWSWIRQPPGKGLEWI
GYIYYSGSTNYNPSLKSRVTISVDTSKNQFSLKLSSVTAADTAVYYCARE
GKNGAFDIWGQGTMVTVSSS

In one embodiment, the human scFV comprises a light chain variable region comprising amino acids 1-107 of SEQ ID NO: 16. In one embodiment, the human scFV comprises a light chain variable region comprising amino acids 1-107 of SEQ ID NO:18.

In certain embodiments, the human scFV comprises amino acids having the sequence set forth below.

(SEQ ID NO: 20)
Q V Q L Q E S G P G L V K P S E T L S L T C T V S
G G S V S S G S Y Y W S W I R Q P P G K G L E W I
G Y I Y Y S G S T N Y N P S L K S R V T I S V D T
S K N Q F S L K L S S V T A A D T A V Y Y C A R E
G K N G A F D I W G Q G T M V T V S S S G G G G S
G G G G S G G G G S R H Q M T Q S P S S L S A S V
G D R V T I T C R A S Q S I S S Y L N W Y Q Q K P
G K A P K L L I Y A A S S L Q S G V P S R F S G S
G S G T D F T L T I S S L Q P E D F A T Y Y C Q Q
S Y S T P L T F G G G T K V E I K G Q A G H H H H
H H G D Y K D D D D K G

In one embodiment, the nucleic acid sequence encoding the amino acid sequence above is set forth below.

(SEQ ID NO: 21)
atggccttaccagtgaccgccttgctcctgccgctggccttgctgctcca
cgccgccaggccgcaggtgcagctgcaggagtccggcccaggactggtga
agccttcggagaccctgtccctcacctgcactgtctctggtggctccgtc
agcagtggtagttactactggagctggatccggcagcccccagggaaggg
actggagtggattgggtatatctattacagtgggagcaccaactacaacc
cctccctcaagagtcgagtcaccatatcagtagacacgtccaagaaccag
ttctccctgaagctgagctctgtgaccgctgcggacacggccgtgtatta
ctgtgcgagagaggggaagaatggggcttttgatatctggggccaaggga
caatggtcaccgtctcttcaggtggaggcggttcaggcggaggtggctct
ggcggtggcggatcacgacatcagatgacccagtctccatcctccctgtc
tgcatctgtaggagacagagtcaccatcacttgccgggcaagtcagagca
ttagcagctatttaaattggtatcagcagaaaccagggaaagcccctaag
ctcctgatctatgctgcatccagtttgcaaagtggggtcccatcaaggtt
cagtggcagtggatctgggacagatttcactctcaccatcagcagtctgc
aacctgaagattttgcaacttactactgtcaacagagttacagtaccccg
ctcactttcggcggagggaccaaggtggagatcaaacggactgcggccgc
a

In another embodiment, a nucleic acid sequence encoding the amino acid sequence of SEQ ID NO:20 is as provided below. The nucleic acid sequence set forth below is synthetically optimized for codon usage, which can increase the expression of the CAR, as disclosed herein.

(SEQ ID NO: 22)
ATGGCGCTGCCGGTGACCGCGCTGCTGCTGCCGCTGGCGCTGCTGCTGCAT
GCGGCGCGCCCGCAGGTGCAGCTGCAGGAAAGCGGCCCGGGCCTGGTGAAA
CCGAGCGAAACCCTGAGCCTGACCTGCACCGTGAGCGGCGGCAGCGTGAGC
AGCGGCAGCTATTATTGGAGCTGGATTCGCCAGCCGCCGGGCAAAGGCCTG
GAATGGATTGGCTATATTTATTATAGCGGCAGCACCAACTATAACCCGAGC
CTGAAAAGCCGCGTGACCATTAGCGTGGATACCAGCAAAAACCAGTTTAGC
CTGAAACTGAGCAGCGTGACCGCGGCGGATACCGCGGTGTATTATTGCGCG
CGCGAAGGCAAAAACGGCGCGTTTGATATTTGGGGCCAGGGCACCATGGTG
ACCGTGAGCAGCGGCGGCGGCGGCAGCGGCGGCGGCGGCAGCGGCGGCGGC
GGCAGCCGCCATCAGATGACCCAGAGCCCGAGCAGCCTGAGCGCGAGCGTG
GGCGATCGCGTGACCATTACCTGCCGCGCGAGCCAGAGCATTAGCAGCTAT
CTGAACTGGTATCAGCAGAAACCGGGCAAAGCGCCGAAACTGCTGATTTAT
GCGGCGAGCAGCCTGCAGAGCGGCGTGCCGAGCCGCTTTAGCGGCAGCGGC
AGCGGCACCGATTTTACCCTGACCATTAGCAGCCTGCAGCCGGAAGATTTT
GCGACCTATTATTGCCAGCAGAGCTATAGCACCCCGCTGACCTTTGGCGGC
GGCACCAAAGTGGAAATTAAACGCACCGCGGCGGCG

In yet another embodiment, a nucleic acid sequence encoding the amino acid sequence of SEQ ID NO:20 is as provided below. The nucleic acid sequence as set forth below is synthetically optimized for codon usage, which can increase the expression of the CAR.

(SEQ ID NO: 23)
atggccCTCCCGGTAACGGCTCTGCTGCTTCCACTCGCACTGCTCTTGCAT
GCTGCCAGACCACAGGTCCAGCTGCAGGAGAGTGGGCCTGGACTGGTTAAG
CCGAGTGAGACACTTTCCTTGACGTGCACTGTGAGCGGGGGAAGTGTGTCC
TCAGGTAGTTATTACTGGTCCTGGATTCGCCAGCCACCAGGAAAGGGACTG
GAGTGGATAGGTTATATCTATTATTCTGGCAGCACTAATTACAATCCTTCT
CTCAAAAGTAGGGTGACAATTTCAGTGGATACTTCCAAAAATCAGTTTAGT
CTGAAGCTCAGCTCTGTGACAGCTGCTGATACTGCAGTTTACTACTGCGCC
AGGGAGGGGAAGAATGGCGCCTTCGATATTTGGGGACAGGGCACTATGGTG
ACTGTATCAAGCGGAGGCGGTGGCAGCGGCGGGGGAGGGAGTGGAGGCGGC
GGGTCTCGACATCAGATGACACAGAGCCCATCATCACTTAGCGCCAGCGTT
GGCGACCGGGTTACGATAACATGCAGGGCTTCCCAATCTATCAGTTCTTAT
CTGAACTGGTATCAGCAGAAACCAGGTAAGGCCCCCAAGCTGCTCATCTAC
GCAGCCTCATCCCTGCAGAGCGGCGTCCCTAGTCGATTTTCCGGTAGTGGG
TCAGGGACAGATTTTACCCTGACTATCAGTTCACTGCAGCCCGAGGACTTC
GCGACATACTATTGCCAACAGTCCTATAGTACACCCTTGACATTTGGCGGC
GGGACTAAAGTAGAAATTAAACGCACCgcggccgca

In certain embodiments, the extracellular antigen-binding domain of a CAR comprises a heavy chain variable region CDR1 comprising the amino acids GGSVSSGSYY (SEQ ID NO:24), a heavy chain variable region CDR2 comprising the amino acids IYYSGST (SEQ ID NO:25), and a heavy chain variable region CDR3 comprising the amino acids AREGKNGAFDIW (SEQ ID NO:26). In some embodiments, the extracellular antigen-binding domain comprises a light chain variable region CDR1 comprising the amino acids QSISSY (SEQ ID NO:27), a light chain variable region CDR2 comprising the amino acids AASS (SEQ ID NO:28), and a light chain variable region CDR3 comprising the amino acids QQSYSTPLTF (SEQ ID NO:29). In one non-limiting, exemplary embodiment, the extracellular antigen-binding domain is a human scFv derived from a fully human anti-MSLN antibody m912 as disclosed in Feng et al., Mol. Cancer Therapy 8(5):1113-1118 (2009), which is incorporated herein by reference.

In one embodiment, an exemplary CAR is Mz, which comprises an extracellular antigen binding domain that specifically binds to human mesothelin, a transmembrane domain comprising a CD8 polypeptide, and an intracellular domain comprising a CD3ζ polypeptide (see FIG. 1B). Mz also comprises a signal peptide covalently joined to the N-terminus of the extracellular antigen-binding domain. The signal peptide comprises a CD8 polypeptide comprising amino acids having the sequence MALPVTALLLPLALLLHAARP (SEQ ID NO:30).

In one embodiment, an exemplary CAR is M28z, which comprises an extracellular antigen binding domain that specifically binds to human mesothelin, a transmembrane domain comprising a CD28 polypeptide, and an intracellular domain comprising a CD3ζ polypeptide and a co-stimulatory signaling region comprising a CD28 polypeptide (see FIG. 1B). M28z also comprises a signal peptide covalently joined to the N-terminus of the extracellular antigen-binding domain. The signal peptide comprises a CD8 polypeptide comprising amino acids having the sequence MALPVTALLLPLALLLHAARP (SEQ ID NO:31).

In one embodiment, an exemplary CAR is MBBz, which comprises an extracellular antigen binding domain that specifically binds to human mesothelin, a transmembrane domain comprising a CD8 polypeptide, and an intracellular domain comprising a CD3ζ polypeptide and a co-stimulatory signaling region comprising a 4-1BB polypeptide (see FIG. 1B). MBBz also comprises a signal peptide covalently joined to the N-terminus of the extracellular antigen-binding domain. The signal peptide comprises a CD8 polypeptide comprising amino acids having the sequence MALPVTALLLPLALLLHAARP (SEQ ID NO:32).

### 6.3. Dominant Negative Forms of an Inhibitor of a Cell-Mediated Immune Response

According to the invention, an immune cell, such as a T cell, or a precursor cell thereof, is engineered to express a dominant negative form (DN form) of an inhibitor of a cell-mediated immune response.

Malignant cells adapt to generate an immunosuppressive microenvironment that protects the cells from immune recognition and elimination (Sharpe et al., Dis. Model Mech. 8:337-350 (2015)). The immunosuppressive microenvironment puts limitations on immunotherapy methods. The present invention addresses this limitation by expressing in an immune cell, or precursor cell thereof, a DN form of an inhibitor of a cell-mediated immune response.

An inhibitor of a cell-mediated immune response of the immune cell or precursor cell thereof refers to a molecule that acts to inhibit or suppress the immune response effected by the immune cell or precursor cell thereof. In one embodiment, the inhibitor of a cell-mediated immune response is an immune checkpoint inhibitor, also referred to as a checkpoint blockade.

In one embodiment, the invention provides immune cells, such as T cells, or precursor cells thereof, that co-express a cancer antigen CAR and a dominant negative form of an inhibitor of a cell-mediated immune response of the immune cell, for example, a receptor that functions in an immune checkpoint inhibitor pathway. Immune checkpoint pathways are inhibitory pathways that suppress the immune response of an immune cell. The pathways deliver negative signals to the immune cells, such as T cells, and attenuate TCR-mediated signals, leading to decreased cell proliferation, cytokine production and cell cycle progression (see Pardoll, Nat. Rev. 12:252-264 (2012); Wu et al., Int. J Biol. Sci. 8:1420-1430 (2012)). The immune checkpoint inhibitor pathway generally involves a ligand-receptor pair. Exemplary immune checkpoint inhibitor pathway receptors include, for example, PD-1, CTLA-4, BTLA, TIM-3, LAG-3, CD160, TIGIT, LAIRI, 2B4, and the like (see Chen et al., Nat. Rev. Immunol. 13(4):227-242 (2013)). The corresponding ligands for these receptors include, for example, PD-L1 (for PD-1); PD-L2 (for PD-1); CD80, CD86 (for CTLA-4); HVEM (for BTLA); Galectin-9, HMGB1 (for TIM-3); MHC II (for LAG-3); HVEM (for CD160); CD155, CD112, CD113 (for TIGIT); C1q, collagen (for LAIR1); CD48 (for 2B4), and the like (Chen et al., supra, 2013). Expression of a DN form in the immune cell, such as a T cell, or precursor cell thereof, provides for inhibition of a checkpoint inhibitor pathway that is intrinsic to the cell.

In one embodiment of the invention, a dominant negative form (“DN form”) of an immune checkpoint inhibitor pathway receptor is provided, as disclosed herein.

A DN form of an inhibitor of a cell-mediated immune response that is a cell-surface receptor such as an immune checkpoint inhibitor pathway receptor can be generated by deleting some portion of the receptor to prevent intracellular signaling, thereby suppressing the immune checkpoint pathway and sustaining activation of the immune cell, such as a T cell. A DN form of the invention is a polypeptide comprising (a) at least a portion of an extracellular domain of an immune checkpoint inhibitor, where the portion comprises the ligand binding region, and (b) a transmembrane domain, where the polypeptide is a dominant negative form of the immune checkpoint inhibitor. Generally, a DN form of an inhibitor of an immune checkpoint inhibitor pathway receptor retains most or all of an extracellular domain of the receptor such that the extracellular domain retains sufficient protein interaction activity to bind to its respective ligand. Thus, in a specific embodiment, a polypeptide encoding a DN form comprises substantially all of an extracellular domain of an immune checkpoint inhibitor. It is understood that a polypeptide comprising “substantially all” of an extracellular domain includes a polypeptide that comprises the entire extracellular domain or a portion of the extracellular domain in which one to a few amino acids have been deleted from the N-terminus and/or C-terminus of the extracellular domain, for example deletion of 1, 2, 3, 4, or 5 amino acids from the N-terminus and/or C-terminus, so long as the remaining portion of the extracellular domain retains sufficient protein interaction activity to bind to its respective ligand. A DN form of the invention generally also lacks some portion or all of a signaling domain, such as the intracellular/cytoplasmic domain, such that the DN form has reduced activity or is inactive for signaling in the immune checkpoint pathway. Without being bound by a particular mechanism or theory, binding of the ligand to the DN form decreases binding of the ligand to the intact endogenous receptor, and/or the DN form complexes with signaling molecules, including the endogenous receptor, resulting in decreased signaling of an immune checkpoint pathway.

A DN form of the invention generally has certain functional characteristics including, but not limited to, the ability to be expressed at the cell surface of an immune cell such as a T cell, or precursor cell thereof, the ability to bind to its respective ligand, and the inability or reduced ability to propagate an intracellular signal of an immune checkpoint pathway. One skilled in the art can readily generate a DN form of an inhibitor of a cell-mediated immune response by engineering the inhibitor to have such functional characteristics. In one embodiment, a DN form is constructed to retain the extracellular domain of inhibitor of a cell-mediated immune response, or at least a sufficient portion of the extracellular domain to retain ligand binding activity. In an exemplary embodiment, a DN form can be constructed using the extracellular domain of an inhibitor of a cell-mediated immune response, including, but not limited to, the extracellular domains of PD-1, CTLA-4, BTLA, TIM-3, LAG-3, CD160, TIGIT, LAIRI, 2B4, as disclosed herein. One skilled in the art will readily understand that it is not required to retain the entire extracellular domain of an inhibitor of a cell-mediated immune response, and that deletions from the N-terminus and/or C-terminus of the extracellular domain can be introduced so long as ligand binding activity is retained. One skilled in the art can readily determine the appropriateness of such N-terminal and/or C-terminal deletions based on the analysis of the receptor sequence to identify protein motifs known to provide ligand binding activity (see, for example, ExPASy (expasy.org), in particular PROSITE (prosite.expasy.org)). In addition or alternatively, suitable N-terminal and/or C-terminal deletions can be determined empirically by introducing deletions in a polypeptide and measuring binding activity for the respective ligand. Thus, one skilled in the art can readily determine an appropriate sequence of an inhibitor of a cell-mediated immune response to provide ligand binding activity to a DN form of the invention.

It is understood that, whether an entire extracellular domain or a portion of the extracellular domain of a receptor is used in a DN form, additional sequences can optionally be included in the extracellular domain of the DN form. Such additional sequences can be derived from the parent polypeptide of the DN form, or the additional sequences can be derived from a different polypeptide. Such a polypeptide comprising sequences from a parent polypeptide and a different polypeptide is a non-naturally occurring, chimeric polypeptide. For example, a signal peptide or leader peptide is generally included so that the DN form will be expressed at the cell surface of the immune cell such as a T cell, or precursor cell thereof. It is understood that, once a polypeptide containing a signal peptide is expressed at the cell surface, the signal peptide has generally been proteolytically removed during processing of the polypeptide in the endoplasmic reticulum and translocation to the cell surface. Thus, a polypeptide such as a DN form is generally expressed at the cell surface as a mature protein lacking the signal peptide, whereas the precursor form of the polypeptide includes the signal peptide. The signal peptide can be the naturally occurring signal peptide of the receptor, or alternatively can be derived from a different protein. Exemplary signal peptides are described herein, including those described herein as being suitable for a CAR. To additionally provide expression at the cell surface, the DN form will generally include a transmembrane domain that provides for retention of the DN form at the cell surface. The transmembrane domain can be the naturally occurring transmembrane of the receptor, or alternatively can be derived from a different protein. In a particular embodiment, the transmembrane domain derived from another protein is derived from another receptor expressed on the cell surface of the immune cell such as a T cell, or precursor cell thereof. Exemplary transmembrane domains are described herein, including those described herein as being suitable for a CAR.

In the case of an immune checkpoint pathway receptor, generally the signaling domain resides within the intracellular/cytoplasmic domain. The signaling activity of an immune checkpoint pathway receptor is generally mediated by protein-protein interactions with cell surface receptor(s) and/or intracellular signaling molecules. In one embodiment, a DN form lacks the entire intracellular domain, or a portion thereof, that functions in propagating the signal of an immune checkpoint pathway. It is understood that it is not necessary to delete the entire intracellular domain of the receptor so long as a sufficient portion of the intracellular signaling domain is deleted to inhibit or reduce signaling from the DN form. In addition or alternatively, mutations can be introduced into the intracellular signaling domain to inhibit or reduce signaling from the DN form. In addition or alternatively, a heterologous sequence with no signaling activity can be substituted for the intracellular signaling domain of the receptor to generate a DN form. One skilled in the art will readily understand that these and other well known methods can be utilized to generate a DN form of the invention.

One exemplary embodiment of a dominant negative form of an immune checkpoint inhibitor is a dominant negative form of PD-1. As disclosed herein, a dominant negative form of PD-1 was co-expressed in a CAR T cell with a mesothelin CAR and found to increase tumor elimination and prolong mouse survival (see Example). A dominant negative form of PD-1 is exemplary of a DN form of an inhibitor of a cell-mediated immune response, including an immune checkpoint inhibitor. The results disclosed herein indicate that co-expressing a dominant negative form of an inhibitor of a cell-mediated immune response can enhance the effectiveness of a CAR T cell, or other immune cell or precursor cell thereof, expressing a cancer antigen CAR. It is understood that a PD-1 DN form as disclosed herein is exemplary. Based on the teachings disclosed herein, one skilled in the art can readily prepare a DN form of an inhibitor of a cell-mediated immune response, including an immune checkpoint pathway receptor.

As described herein, a DN form of an inhibitor of a cell-mediated immune response is designed to have reduced or inhibited intracellular signaling. The DN forms of the invention are generally based on inhibiting a receptor of an immune checkpoint pathway, which function to inhibit activation of an immune cell, such as T cell, for example, cell proliferation, cytokine production and/or cell cycle progression. The DN forms of the invention are designed to remove the intracellular signaling domain, or a portion thereof, so that the signaling ability of the receptor is reduced or inhibited. The DN form also functions to inhibit signaling of the endogenous receptor. In a particular embodiment, the reduced or inhibited signaling overcomes the checkpoint blockade, resulting in sustained signaling and activation of the immune cell, such as a T cell, or precursor cell thereof. It is understood that the signaling activity of the DN form can be completely knocked out or partially knocked out, so long as the partial reduction in activity is sufficient for the effect of providing enhanced activation of the immune cell, or precursor cell thereof, in comparison to the absence of the DN form. Also, the DN form is not required to result in complete inactivation of signaling from the endogenous receptor but can reduce the activation of the endogenous receptor sufficient to overcome the checkpoint blockade and allow activation of the immune cell, such as a T cell, or precursor cell thereof. One skilled in the art can readily determine the effect of a DN form on the activity of a parent receptor using assay methods well known in the art, including assays using in vivo models, such as animal models, to assess the effect of the DN form on the effectiveness of CAR expressing cells, as disclosed herein.

As with a CAR for use in the invention, optional linker or spacer sequences can be included in a DN form, for example, a linker or spacer between a signal peptide and the extracellular ligand binding domain, particularly when heterologous sequences are fused. A linker or spacer can also optionally be included between the extracellular ligand binding domain and the transmembrane domain. Similarly, a linker or spacer can optionally be included between the transmembrane domain and any remaining intracellular domain. Such optional linkers or spacers are described herein. In addition, such linkers or spacers can be derived from a heterologous sequence. For example, as described above, a transmembrane domain derived from a heterologous polypeptide can optionally include additional sequences at the N-terminus and/or C-terminus derived from the heterologous polypeptide. Such additional sequences can function as a linker or spacer.

Exemplary DN forms of immune checkpoint inhibitors are described below in more detail. DN forms consisting essentially of the described sequences are also envisioned.

PD-1. Programmed cell death protein 1 (PD-1) is a negative immune regulator of activated T cells upon engagement with its corresponding ligands, PD-L1 and PD-L2, expressed on endogenous macrophages and dendritic cells. PD-1 is a type 1 membrane protein of 268 amino acids. PD-1 has two ligands, PD-L1 and PD-L2, which are members of the B7 family. The protein's structure comprises an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif. PD-1 negatively regulates TCR signals. SHP-1 and SHP-2 phosphatases bind to the cytoplasmic tail of PD-1 upon ligand binding. Upregulation of PD-L1 is one mechanism tumor cells use to evade the host immune system. In pre-clinical and clinical trials, PD-1 blockade by antagonistic antibodies induced anti-tumor responses mediated through the host endogenous immune system.

A PD-1 polypeptide can have an amino acid corresponding to GenBank No. NP_005009.2 (GI:167857792), as provided below, or fragments thereof. See GenBank NP_005009.2 for reference to domains within PD-1, for example, signal peptide, amino acids 1 to 20; extracellular domain, amino acids 21 to 170; transmembrane domain, amino acids 171 to 191; intracellular domain, amino acids 192 to 288. It is understood that an “PD-1 nucleic acid molecule” refers to a polynucleotide encoding an PD-1 polypeptide.

| (NP_005009.2; SEQ ID NO: 33) | |
|---|---|
| 1 | MQIPQAPWPV VWAVLQLGWR PGWFLDSPDR PWNPPTFSPA LLVVTEGDNA TFTCSFSNTS |
| 61 | ESFVLNWYRM SPSNQTDKLA AFPEDRSQPG QDCRFRVTQL PNGRDFHMSV VRARRNDSGT |
| 121 | YLCGAISLAP KAQIKESLRA ELRVTERRAE VPTAHPSPSP RPAGQFQTLV VGVVGGLLGS |
| 181 | LVLLVWVLAV ICSRAARGTI GARRTGQPLK EDPSAVPVFS VDYGELDFQW REKTPEPPVP |
| 241 | CVPEQTEYAT IVFPSGMGTS SPARRGSADG PRSAQPLRPE DGHCSWPL |

In one embodiment, the invention provides an inhibitor of a cell-mediated immune response that is a PD-1 dominant negative form (DN form). In one embodiment, the PD-1 DN form comprises the extracellular ligand binding domain of PD-1. In one embodiment, the PD-1 DN form comprises the extracellular ligand binding domain of PD-1 and a transmembrane domain (e.g., mature form). In another embodiment, the PD-1 DN form comprises the extracellular ligand binding domain of PD-1, a transmembrane domain and a signal peptide (e.g., precursor form). The invention also provides encoding polypeptides and nucleic acids of the PD-1 DN forms of the invention. In a particular embodiment, the PD-1 extracellular ligand binding domain is fused to one or more heterologous polypeptide sequences, that is, the PD-1 DN form is a chimeric sequence. For example, the PD-1 extracellular ligand binding domain can be fused at its N-terminus to a signal peptide that is optionally a heterologous signal peptide, including various signal peptides described herein. In addition, a PD-1 DN form can comprise a transmembrane domain that is optionally a heterologous transmembrane domain, including any of various transmembrane domains described herein. Although the PD-1 DN form exemplified in the Example herein comprises heterologous sequences fused to the extracellular domain of PD-1, it is understood that a PD-1 DN form can comprise PD-1 sequence only.

In one embodiment, the invention provides a PD-1 DN form that comprises the extracellular domain, or a ligand binding portion thereof, of PD-1, for example, amino acids 21 to 170 corresponding to the extracellular domain of PD-1 (GenBank NP_005009.2; SEQ ID NO:33). A cell expressing such a PD-1 DN form should lack the ability or have reduced ability to signal in a PD-1 immune checkpoint pathway. In one embodiment, a PD-1 DN form is a deletion mutant having a deletion of the intracellular domain, for example, amino acids 192 to 288 of PD-1 (GenBank NP_005009.2; SEQ ID NO:33), or a portion thereof, such that intracellular signaling of the immune checkpoint pathway mediated by PD-1 is reduced or inhibited. Additional embodiments of a DN form of PD-1 are described below.

In one embodiment, a PD-1 DN form comprises an amino acid sequence comprising the extracellular domain of PD-1 fused to the transmembrane and hinge domains of CD8. In one embodiment, a PD-1 DN form comprises amino acids 21 to 165 of a PD-1 sequence (NP_005009.2; SEQ ID NO:33). Such a PD-1 DN form comprises the extracellular domain of PD-1. In another embodiment, the invention provides a PD-1 DN form comprising amino acids 1 to 165 (precursor form) or amino acids 21 to 165 (mature form) of a PD-1 sequence (NP_005009.2; SEQ ID NO:33). Such a DN form comprises the signal peptide of PD-1, amino acids 1 to 20, and extracellular domain amino acids 21 to 165, whereas the mature form lacks the signal peptide. In one embodiment, a PD-1 DN form comprises amino acids 21 to 151 of a PD-1 sequence (NP_005009.2; SEQ ID NO:33). In another embodiment, the invention provides a PD-1 DN form comprising amino acids 1 to 151 (precursor form) or amino acids 21 to 151 (mature form) of a PD-1 sequence (NP_005009.2; SEQ ID NO:33). Optionally, a PD-1 DN form comprises an extracellular ligand binding domain starting at amino acid 21 through an amino acid between amino acids 151 to 165 of a PD-1 sequence (NP_005009.2; SEQ ID NO:33). In another embodiment, a PD-1 DN form comprises the transmembrane domain of CD8, amino acids 183 to 203 of a CD8 sequence (NP_001139345.1; SEQ ID NO:11). Such an embodiment is representative of a chimeric DN form comprising a transmembrane domain from a different (heterologous) polypeptide. As described above, a DN form comprising a heterologous domain such as a transmembrane domain can optionally include additional sequence from the heterologous polypeptide. In one such embodiment, a DN form is provided that comprises additional sequence from the heterologous polypeptide N-terminal of the transmembrane domain. In one embodiment, the DN form comprises the hinge domain of CD8. In a particular embodiment, the heterologous sequence comprises additional N-terminal sequence of amino acids 137 to 182, or optionally starting at amino acids 138 or 139, of a CD8 sequence (NP_001139345.1; SEQ ID NO: 11). In another embodiment, a DN form is provided that comprises additional sequence from the heterologous polypeptide C-terminal of the transmembrane domain. In a particular embodiment, the heterologous sequence comprises additional C-terminal sequence from amino acids 204 to 209 of a CD8 sequence (NP_001139345.1; SEQ ID NO:11). In one embodiment, the PD-1 DN form comprises the transmembrane domain of CD8, amino acids 183 to 203, optionally a hinge domain comprising amino acids 137 to 182 (or optionally starting at amino acids 138 or 139), and/or additional C-terminal sequence comprising amino acids 204 to 209. In a particular embodiment of the invention, a PD-1 DN form is provided that comprises amino acids 1 to 165 of a PD-1 sequence (NP_005009.2; SEQ ID NO:33), and amino acids 137 to 209, optionally starting at amino acids 138 or 139, of a CD8 sequence (NP_001139345.1; SEQ ID NO:11).

In a further particular embodiment, the invention provides a PD-1 DN form comprising the sequence provided below, where the underlined sequence is derived from PD-1 and the italicized sequence is derived from CD8.

(SEQ ID NO: 43)
MQIPQAPWPVVWAVLQLGWRPGWFLDSPDRPWNPPTFSPALLVVTEGDNAT
FTCSFSNTSESFVLNWYRMSPSNQTDKLAAFPEDRSQPGQDCRFRVTQLPN
GRDFHMSVVRARRNDSGTYLCGAISLAPKAQIKESLRAELRVTERRAEVPT
ARPSPSPRPAGQAAAPTTTPAPRPPTPAPTIASQPLSLRPEACRPAAGGAV
HTRGLDFACDIYIWAPLAGTCGVLLLSLVITLYCNHRRIQ

In an additional embodiment, a DN form of the invention optionally comprises a P2A sequence, which provides for optional co-expression of a reporter molecule. P2A is a sequence used for bicistronic or multicistronic expression of protein sequences (see Szymczak et al., Expert Opin. Biol. Therapy 5(5):627-638 (2005)). An exemplary P2A sequence is GSGATNFSLLKQAGDVEENPGPM (SEQ ID NO:44). In a further embodiment, a DN form of the invention is co-expressed with a reporter protein. In a particular embodiment, the reporter protein is mCherry fluorescent protein. In a particular embodiment, the mCherry polypeptide sequence is as provided below. It is understood that mCherry is merely exemplary and that any desired reporter molecule, such as a fluorescent protein can be included as a reporter, as described herein.

(SEQ ID NO: 45)
MVSKGEEDNMAIIKEFMRFKVHMEGSVNGHEFEIEGEGEGRPYEGTQTAKL
KVTKGGPLPFAWDILSPQFMYGSKAYVKHPADIPDYLKLSFPEGFKWERVM
NFEDGGVVTVTQDSSLQDGEFIYKVKLRGTNFPSDGPVMQKKTMGWEASSE
RMYPEDGALKGEIKQRLKLKDGGHYDAEVKTTYKAKKPVQLPGAYNVNIKL
DITSHNEDYTIVEQYERAEGRHSTGGMDELYK

In a further particular embodiment, a PD-1 DN form is expressed as a polypeptide construct as provided below, where the underlined sequence is derived from PD-1, the italicized sequence is derived from CD8, the P2A sequence is double underlined, and the mCherry sequence is underlined and italicized.

(SEQ ID NO: 46)
MQIPQAPWPVVWAVLQLGWRPGWFLDSPDRPWNPPTFSPALLVVTEGDNAT
FTCSFSNTSESFVLNWYRMSPSNQTDKLAAFPEDRSQPGQDCRFRVTQLPN
GRDFHMSVVRARRNDSGTYLCGAISLAPKAQIKESLRAELRVTERRAEVPT
AHPSPSPRPAGQAAAPTTTPAPRPPTPAPTIASQPLSLRPEACRPAAGGAV
HTRGLDFACDIYIWAPLAGTCGVLLLSLVITLYCNHRRIQGSGATNFSLLK
QAGDVEENPGPMVSKGEEDNMAIIKEFMRFKVHMEGSVNGHEFEIEGEGEG
RPYEGTQTAKLKVTKGGPLPFAWDILSPQFMYGSKAYVKHPADIPDYLKLS
FPEGFKWERVMNFEDGGVVTVTQDSSLQDGEFIYKVKLRGTNFPSDGPMQK
KTMGWEASSERMYPEDGALKGEIKQRLKLKDGGHYDAEVKTTYKAKKPVQL
PGAYNVNIKLDITSHNEDYTIVEQYERAEGRHSTGGMDELYK

In a particular embodiment, a nucleic acid encoding a PD-1 DNR form construct is provided below, where the underlined sequence encodes amino acids derived from PD-1 DN, the italicized sequence encodes amino acids derived from CD8, the P2A encoding sequence is double underlined, the mCherry encoding sequence is underlined and italicized, a Kozak sequence is bolded with a dashed underline, and restriction sites Age I and Xho I are underlined with a dotted line at the 5′ and 3′ ends, respectively.

(SEQ ID NO: 47)

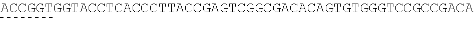

CCAGACTAAGAACCTAGAACCTCGCTGGAAAGGACCTTACACAGTCCTGCTGAC
CACCCCCACCGCCCTCAAAGTAGACGGCATCGCAGCTTGGATACACGCCGCCCA

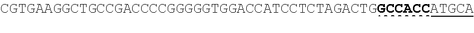

GATCCCACAGGCGCCCTGGCCAGTCGTCTGGGCGGTGCTACAACTGGGCTGGCG
GCCAGGATGGTTCTTAGACTCCCCAGACAGGCCCTGGAACCCCCCCACCTTCTCC
CCAGCCCTGCTCGTGGTGACCGAAGGGGACAACGCCACCTTCACCTGCAGCTTCT
CCAACACATCGGAGAGCTTCGTGCTAAACTGGTACCGCATGAGCCCCAGCAACC
AGACGGACAAGCTGGCCGCTTTCCCCGAGGACCGCAGCCAGCCCGGCCAGGACT
GCCGCTTCCGTGTCACACAACTGCCCAACGGGCGTGACTTCCACATGAGCGTGGT
CAGGGCCCGGCGCAATGACAGCGGCACCTACCTCTGTGGGGCCATCTCCCTGGC
CCCCAAGGCGCAGATCAAAGAGAGCCTGCGGGCAGAGCTCAGGGTGACAGAGA
GAAGGGCAGAAGTGCCCACAGCCCACCCCAGCCCCTCACCCAGGCCAGCCGGCC
AGGCGGCCGCACCCACCACGACGCCAGCGCCGCGACCACCAACCCCGGCGCCCAC
GATCGCGTCGCAGCCCCTGTCCCTGCGCCCAGAGGCGTGCCGGCCAGCGGCGGGG
GGCGCAGTGCACACGAGGGGGCTGGACTTCGCCTGTGATATCTACATCTGGGCGCCC
CTGGCCGGGACTTGTGGGGTCCTTCTCCTGTCACTGGTTATCACCCTTTACTGCAACC
ACAGGCGGATCCAAGGATCTGGAGCAACAAACTTCTCACTACTCAAACAAGCAG
GTGACGTGGAGGAGAATCCCGGCCCCATGGTGAGCAAGGGCGAGGAGGATAACAT
GGCCATCATCAAGGAGTTCATGCGCTTCAAGGTGCACATGGAGGGCTCCGTGAACGG
CCACGAGTTCGAGATCGAGGGCGAGGGCGAGGGCCGCCCCTACGAGGGCACCCAGA
CCGCCAAGCTGAAGGTGACCAAGGGTGGCCCCCTGCCCTTCGCCTGGGACATCCTGT
CCCCTCAGTTCATGTACGGCTCCAAGGCCTACGTGAAGCACCCCGCCGACATCCCCG
ACTACTTGAAGCTGTCCTTCCCCGAGGGCTTCAAGTGGGAGCGCGTGATGAACTTCGA
GGACGGCGGCGTGGTGACCGTGACCCAGGACTCCTCCCTGCAGGACGGCGAGTTCA
TCTACAAGGTGAAGCTGCGCGGCACCAACTTCCCCTCCGACGGCCCCGTAATGCAGA
AGAAGACCATGGGCTGGGAGGCCTCCTCCGAGCGGATGTACCCGAGGACGGCGCC
CTGAAGGGCGAGATCAAGCAGAGGCTGAAGCTGAAGGACGGCGGCCACTACGACGC
TGAGGTCAAGACCACCTACAAGGCCAAGAAGCCCGTGCAGCTGCCCGGCGCCTACAA
CGTCAACATCAAGTTGGACATCACCTCCCACAACGAGGACTACACCATCGTGGAACAG
TACGAACGCGCCGAGGGCCGCCACTCCACGGCGGCATATGGACGAGCTGTACAAGTA

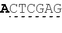

CTLA-4. Cytotoxic T-lymphocyte-associated protein 4 (CTLA-4) is an inhibitory receptor expressed by activated T cells, which when engaged by its corresponding ligands (CD80 and CD86; B7-1 and β7-2, respectively), mediates activated T cell inhibition or anergy. In both preclinical and clinical studies, CTLA-4 blockade by systemic antibody infusion enhanced the endogenous anti-tumor response albeit, in the clinical setting, with significant unforeseen toxicities. CTLA-4 contains an extracellular V domain, a transmembrane domain, and a cytoplasmic tail. Alternate splice variants, encoding different isoforms, have been characterized. The membrane-bound isoform functions as a homodimer interconnected by a disulfide bond, while the soluble isoform functions as a monomer. The intracellular domain is similar to that of CD28, in that it has no intrinsic catalytic activity and contains one YVKM motif able to bind P13K, PP2A and SHP-2 and one proline-rich motif able to bind SH3 containing proteins. One role of CTLA-4 in inhibiting T cell responses seems to be directly via SHP-2 and PP2A dephosphorylation of TCR-proximal signaling proteins such as CD3 and LAT. CTLA-4 can also affect signaling indirectly via competing with CD28 for CD80/86 binding. CTLA-4 has also been shown to bind and/or interact with PI3K, CD80, AP2M1, and PPP2R5A.

A CTLA-4 polypeptide can have an amino acid sequence corresponding to GenBank No. AAH69566.1 (GI:46854814) or NP_005205.2 (GI:21361212), sequence as provided below, or fragments thereof. See GenBank NP_005205.2 for reference to domains within CTLA-4, for example, signal peptide, amino acids 1 to 35; extracellular domain, amino acids 36 to 161; transmembrane domain, amino acids 162 to 182; intracellular domain, amino acids 183 to 223. It is understood that a “CTLA-4 nucleic acid molecule” refers to a polynucleotide encoding a CTLA-4 polypeptide.

| (NP_005205.2; SEQ ID NO: 34) | |
|---|---|
| 1 | MACLGFQRHK AQLNLATRTW PCTLLFFLLF IPVFCKAMHV AQPAVVLASS RGIASFVCEY |
| 61 | ASPGKATEVR VTVLRQADSQ VTEVCAATYM MGNELTFLDD SICTGTSSGN QVNLTIQGLR |
| 121 | AMDTGLYICK VELMYPPPYY LGIGNGTQIY VIDPEPCPDS DFLLWILAAV SSGLFFYSFL |
| 181 | LTAVSLSKML KKRSPLTTGV YVKMPPTEPE CEKQFQPYFI PIN |

In one embodiment, the invention provides a CTLA-4 DN form. In one embodiment, the CTLA-4 DN form comprises the extracellular ligand binding domain of CTLA-4. In one embodiment, the CTLA-4 DN form comprises the extracellular ligand binding domain of CTLA-4 and a transmembrane domain (e.g., mature form). In another embodiment, the CTLA-4 DN form comprises the extracellular ligand binding domain of CTLA-4, a transmembrane domain and a signal peptide (e.g., precursor form). The invention also provides encoding polypeptides and nucleic acids of the CTLA-4 DN forms of the invention. In a particular embodiment, the CTLA-4 extracellular ligand binding domain is fused to one or more heterologous polypeptide sequences, that is, the CTLA-4 DN form is chimeric. For example, the CTLA-4 extracellular ligand binding domain can be fused at its N-terminus to a signal peptide that is optionally a heterologous signal peptide, including various signal peptides described herein. In addition, a CTLA-4 DN form can comprise a transmembrane domain that is optionally a heterologous transmembrane domain, including any of various transmembrane domains described herein.

In an embodiment of the invention, the CTLA-4 DN form can comprise the extracellular domain, or a ligand binding portion thereof, of CTLA-4, for example, amino acids 36 to 161 corresponding to the extracellular domain of CTLA-4 (GenBank NP_005205.2; SEQ ID NO:34). A cell expressing such a CTLA-4 DN form should lack the ability or have reduced ability to signal in a CTLA-4 immune checkpoint pathway. In one embodiment, a CTLA-4 DN form is a deletion mutant having a deletion of the intracellular domain, for example, amino acids 183 to 223 of CTLA-4 (GenBank NP_005205.2; SEQ ID NO:34), or a portion thereof, such that intracellular signaling of the immune checkpoint pathway mediated by CTLA-4 is reduced or inhibited.

BTLA. B- and T-lymphocyte attenuator (BTLA) expression is induced during activation of T cells, and BTLA remains expressed on Th1 cells but not Th2 cells. BTLA interacts with a B7 homolog, B7H4. BTLA displays T-Cell inhibition via interaction with tumor necrosis family receptors (TNF-R), not just the B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses. BTLA activation has been shown to inhibit the function of human CD8⁺ cancer-specific T cells. BTLA has also been designated as CD272 (cluster of differentiation 272).

A BTLA polypeptide can have an amino acid sequence corresponding to GenBank No. AAP44003.1 (GI:31880027) or NP_861445.3 (GI:145580621), sequence provided below, or fragments thereof See GenBank NP_861445.3 for reference to domains within BTLA, for example, signal peptide, amino acids 1 to 30; extracellular domain, amino acids 31 to 157; transmembrane domain, amino acids 158 to 178; intracellular domain, amino acids 179 to 289. It is understood that a “BTLA nucleic acid molecule” refers to a polynucleotide encoding a BTLA polypeptide.

| (NP_861445.3; SEQ ID NO: 35) | |
|---|---|
| 1 | MKTLPAMLGT GKLFWVFFLI PYLDIWNIHG KESCDVQLYI KRQSEHSILA GDPFELECPV |
| 61 | KYCANRPHVT WCKLNGTTCV KLEDRQTSWK EEKNISFFIL HFEPVLPNDN GSYRCSANFQ |
| 121 | SNLIESHSTT LYVTDVKSAS ERPSKDEMAS RPWLLYSLLP LGGLPLLITT CFCLFCCLRR |
| 181 | HQGKQNELSD TAGREINLVD AHLKSEQTEA STRQNSQVLL SETGIYDNDP DLCFRMQEGS |
| 241 | EVYSNPCLEE NKPGIVYASL NHSVIGPNSR LARNVKEAPT EYASICVRS |

In one embodiment, the invention provides a BTLA DN form. In one embodiment, the BTLA DN form comprises the extracellular ligand binding domain of BTLA. In one embodiment, the BTLA DN form comprises the extracellular ligand binding domain of BTLA and a transmembrane domain (e.g., mature form). In another embodiment, the BTLA DN form comprises the extracellular ligand binding domain of BTLA, a transmembrane domain and a signal peptide (e.g., precursor form). The invention also provides encoding polypeptides and nucleic acids of the BTLA DN forms of the invention. In a particular embodiment, the BTLA extracellular ligand binding domain is fused to one or more heterologous polypeptide sequences, that is, the BTLA DN form is chimeric. For example, the BTLA extracellular ligand binding domain can be fused at its N-terminus to a signal peptide that is optionally a heterologous signal peptide, including various signal peptides described herein. In addition, a BTLA DN form can comprise a transmembrane domain that is optionally a heterologous transmembrane domain, including any of various transmembrane domains described herein.

In an embodiment of the invention, the BTLA DN form can comprise the extracellular domain, or a ligand binding portion thereof, of BTLA, for example, amino acids 31 to 157 corresponding to the extracellular domain of BTLA (GenBank NP_861445.3; SEQ ID NO:35). A cell expressing such a BTLA DN form should lack the ability or have reduced ability to signal in a BTLA immune checkpoint pathway. In one embodiment, a BTLA DN form is a deletion mutant having a deletion of the intracellular domain, for example, amino acids 179 to 289 of BTLA (GenBank NP_861445.3; SEQ ID NO:35), or a portion thereof, such that intracellular signaling of the immune checkpoint pathway mediated by BTLA is reduced or inhibited.

TIM-3. T cell immunoglobulin mucin-3 (TIM-3), also referred to as hepatitis A virus cellular receptor 2 precursor, is a Th1-specific cell surface protein that regulates macrophage activation. Tim-3 was first identified as a molecule selectively expressed on IFN-7-producing CD4⁺ T helper 1 (Th1) and CD8⁺ T cytotoxic 1 (Tc1) T cells. TIM-3 possess an N-terminal Ig domain of the V type, followed by a mucin domain.

A TIM-3 polypeptide can have an amino acid sequence corresponding to GenBank No. NP_116171.3 (GI:49574534), sequence provided below, or fragments thereof. See GenBank NP_116171.3 for reference to domains within TIM-3, for example, signal peptide, amino acids 1 to 21; extracellular domain, amino acids 22 to 202; transmembrane domain, amino acids 203 to 223; intracellular domain, amino acids 224 to 301. It is understood that a “TIM-3 nucleic acid molecule” refers to a polynucleotide encoding a TIM-3 polypeptide.

| (NP_116171.3; SEQ ID NO: 36) | |
|---|---|
| 1 | MFSHLPFDCV LLLLLLLLTR SSEVEYRAEV GQNAYLPCFY TPAAPGNLVP VCWGKGACPV |
| 61 | FECGNVVLRT DERDVNYWTS RYWLNGDFRK GDVSLTIENV TLADSGIYCC RIQIPGIMND |
| 121 | EKFNLKLVIK PAKVTPAPTR QRDFTAAFPR MLTTRGHGPA ETQTLGSLPD INLTQISTLA |
| 181 | NELRDSRLAN DLRDSGATIR IGIYIGAGIC AGLALALIFG ALIFKWYSHS KEKIQNLSLI |
| 241 | SLANLPPSGL ANAVAEGIRS EENIYTIEEN VYEVEEPNEY YCYVSSRQQP SQPLGCRFAM |
| 301 | P |

In one embodiment, the invention provides a TIM-3 DN form. In one embodiment, the TIM-3 DN form comprises the extracellular ligand binding domain of TIM-3. In one embodiment, the TIM-3 DN form comprises the extracellular ligand binding domain of TIM-3 and a transmembrane domain (e.g., mature form). In another embodiment, the TIM-3 DN form comprises the extracellular ligand binding domain of TIM-3, a transmembrane domain and a signal peptide (e.g., precursor form). The invention also provides encoding polypeptides and nucleic acids of the TIM-3 DN forms of the invention. In a particular embodiment, the TIM-3 extracellular ligand binding domain is fused to one or more heterologous polypeptide sequences, that is, the TIM-3 DN form is chimeric. For example, the TIM-3 extracellular ligand binding domain can be fused at its N-terminus to a signal peptide that is optionally a heterologous signal peptide, including various signal peptides described herein. In addition, a TIM-3 DN form can comprise a transmembrane domain that is optionally a heterologous transmembrane domain, including any of various transmembrane domains described herein.

In an embodiment of the invention, the TIM-3 DN form can comprise the extracellular domain, or a ligand binding portion thereof, of TIM-3, for example, amino acids 22 to 202 corresponding to the extracellular domain of TIM-3 (GenBank NP_116171.3; SEQ ID NO:36). A cell expressing such a TIM-3 DN form should lack the ability or have reduced ability to signal in a TIM-3 immune checkpoint pathway. In one embodiment, a TIM-3 DN form is a deletion mutant having a deletion of the intracellular domain, for example, amino acids 224 to 301 of TIM-3 (GenBank NP_116171.3; SEQ ID NO:36), or a portion thereof, such that intracellular signaling of the immune checkpoint pathway mediated by TIM-3 is reduced or inhibited.

LAG-3. Lymphocyte-activation protein 3 (LAG-3) is a negative immune regulator of immune cells. LAG-3 belongs to the immunoglobulin (Ig) superfamily and contains 4 extracellular Ig-like domains. The LAG3 gene contains 8 exons. The sequence data, exon/intron organization, and chromosomal localization all indicate a close relationship of LAG-3 to CD4. LAG-3 has also been designated CD223 (cluster of differentiation 223).

A LAG-3 polypeptide can have an amino acid sequence corresponding to GenBank No. CAA36243.3 (GI:15617341) or NP_002277.4 (GI:167614500), sequence provided below, or fragments thereof See GenBank NP_002277.4 for reference to domains within LAG-3, for example, signal peptide, amino acids 1 to 22; extracellular domain, amino acids 23 to 450; transmembrane domain, amino acids 451 to 471; intracellular domain, amino acids 472 to 525. It is understood that a “LAG-3 nucleic acid molecule” refers to a polynucleotide encoding a LAG-3 polypeptide.

| (NP_002277.4; SEQ ID NO: 37) | |
|---|---|
| 1 | MWEAQFLGLL FLQPLWVAPV KPLQPGAEVP VVWAQEGAPA QLPCSPTIPL QDLSLLRRAG |
| 61 | VTWQHQPDSG PPAAAPGHPL APGPHPAAPS SWGPRPRRYT VLSVGPGGLR SGRLPLQPRV |
| 121 | QLDERGRQRG DFSLWLRPAR RADAGEYRAA VHLRDRALSC RLRLRLGQAS MTASPPGSLR |
| 181 | ASDWVILNCS FSRPDRPASV HWFRNRGQGR VPVRESPHHH LAESFLFLPQ VSPMDSGPWG |
| 241 | CILTYRDGFN VSIMYNLTVL GLEPPTPLTV YAGAGSRVGL PCRLPAGVGT RSFLTAKWTP |
| 301 | PGGGPDLLVT GDNGDFTLRL EDVSQAQAGT YTCHIHLQEQ QLNATVTLAI ITVTPKSFGS |
| 361 | PGSLGKLLCE VTPVSGQERF VWSSLDTPSQ RSFSGPWLEA QEAQLLSQPW QCQLYQGERL |
| 421 | LGAAVYFTEL SSPGAQRSGR APGALPAGHL LLFLILGVLS LLLLVTGAFG FHLWRRQWRP |
| 481 | RRFSALEQGI HPPQAQSKIE ELEQEPEPEP EPEPEPEPEP EPEQL |

In one embodiment, the invention provides a LAG-3 DN form. In one embodiment, the LAG-3 DN form comprises the extracellular ligand binding domain of LAG-3. In one embodiment, the LAG-3 DN form comprises the extracellular ligand binding domain of LAG-3 and a transmembrane domain (e.g., mature form). In another embodiment, the LAG-3 DN form comprises the extracellular ligand binding domain of LAG-3, a transmembrane domain and a signal peptide (e.g., precursor form). The invention also provides encoding polypeptides and nucleic acids of the LAG-3 DN forms of the invention. In a particular embodiment, the LAG-3 extracellular ligand binding domain is fused to one or more heterologous polypeptide sequences, that is, the LAG-3 DN form is chimeric. For example, the LAG-3 extracellular ligand binding domain can be fused at its N-terminus to a signal peptide that is optionally a heterologous signal peptide, including various signal peptides described herein. In addition, a LAG-3 DN form can comprise a transmembrane domain that is optionally a heterologous transmembrane domain, including any of various transmembrane domains described herein.

In an embodiment of the invention, the LAG-3 DN form can comprise the extracellular domain, or a ligand binding portion thereof, of LAG-3, for example, amino acids 23 to 450 corresponding to the extracellular domain of LAG-3 (GenBank NP_002277.4; SEQ ID NO:37). A cell expressing such a LAG-3 DN form should lack the ability or have reduced ability to signal in a LAG-3 immune checkpoint pathway. In one embodiment, a LAG-3 DN form is a deletion mutant having a deletion of the intracellular domain, for example, amino acids 472 to 525 of LAG-3 (GenBank NP_002277.4; SEQ ID NO:37), or a portion thereof, such that intracellular signaling of the immune checkpoint pathway mediated by LAG-3 is reduced or inhibited.

TIGIT. T-cell immunoreceptor with Ig and ITIM domains (TIGIT) is a cell surface protein that suppresses T-cell activation. It belongs to the poliovirus receptor (PVR) family of immunoglobulin (Ig) proteins that share 3 conserved sequence motifs in their N-terminal Ig domains. A TIGIT polypeptide can have an amino acid sequence corresponding to GenBank No. NP_776160.2 (GI:256600228), sequence provided below, or fragments thereof. See GenBank NP_776160.2 for reference to domains within TIGIT, for example, signal peptide, amino acids 1 to 21; extracellular domain, amino acids 22 to 141; transmembrane domain, amino acids 142 to 162; intracellular domain, amino acids 163 to 244. It is understood that a “TIGIT nucleic acid molecule” refers to a polynucleotide encoding a TIGIT polypeptide.

| (NP_776160.2; SEQ ID NO: 38) | |
|---|---|
| 1 | MRWCLLLIWA QGLRQAPLAS GMMTGTIETT GNISAEKGGS IILQCHLSST TAQVTQVNWE |
| 61 | QQDQLLAICN ADLGWHISPS FKDRVAPGPG LGLTLQSLTV NDTGEYFCIY HTYPDGTYTG |
| 121 | RIFLEVLESS VAEHGARFQI PLLGAMAATL VVICTAVIVV VALTRKKKAL RIHSVEGDLR |
| 181 | RKSAGQEEWS PSAPSPPGSC VQAEAAPAGL CGEQRGEDCA ELHDYFNVLS YRSLGNCSFF |
| 241 | TETG |

In one embodiment, the invention provides a TIGIT DN form. In one embodiment, the TIGIT DN form comprises the extracellular ligand binding domain of TIGIT. In one embodiment, the TIGIT DN form comprises the extracellular ligand binding domain of TIGIT and a transmembrane domain (e.g., mature form). In another embodiment, the TIGIT DN form comprises the extracellular ligand binding domain of TIGIT, a transmembrane domain and a signal peptide (e.g., precursor form). The invention also provides encoding polypeptides and nucleic acids of the TIGIT DN forms of the invention. In a particular embodiment, the TIGIT extracellular ligand binding domain is fused to one or more heterologous polypeptide sequences, that is, the TIGIT DN form is chimeric. For example, the TIGIT extracellular ligand binding domain can be fused at its N-terminus to a signal peptide that is optionally a heterologous signal peptide, including various signal peptides described herein. In addition, a TIGIT DN form can comprise a transmembrane domain that is optionally a heterologous transmembrane domain, including any of various transmembrane domains described herein.

In an embodiment of the invention, the TIGIT DN form can comprise the extracellular domain, or a ligand binding portion thereof, of TIGIT, for example, amino acids 22 to 141 corresponding to the extracellular domain of TIGIT (GenBank NP_776160.2; SEQ ID NO:38). A cell expressing such a TIGIT DN form should lack the ability or have reduced ability to signal in a TIGIT immune checkpoint pathway. In one embodiment, a TIGIT DN form is a deletion mutant having a deletion of the intracellular domain, for example, amino acids 163 to 244 of TIGIT (GenBank NP_776160.2; SEQ ID NO:38), or a portion thereof, such that intracellular signaling of the immune checkpoint pathway mediated by TIGIT is reduced or inhibited.

LAIR1. Leukocyte-associated immunoglobulin-like receptor 1 (LAIR1) is an inhibitory receptor that plays a constitutive negative regulatory role on cytolytic function of natural killer (NK) cells, B-cells and T-cells. LAIR exists in various isoforms. It is understood that any isoform can be selected to achieve a desired function. Exemplary isoforms include isoform a (NP_002278.2, GI:612407859), isoform b (NP_068352.2, GI:612407861), isoform c (NP_001275952.2, GI:612407867), isoform e (NP_001275954.2, GI:612407869), isoform f (NP_001275955.2, GI:612407863), isoform g (NP_001275956.2, GI:612407865), and the like. One exemplary isoform sequence, isoform a, is provided below. In one embodiment, a LAIRI polypeptide can have an amino acid sequence corresponding to NP_002278.2, sequence provided below, or fragments thereof. See GenBank NP_002278.2 for reference to domains within LAIR1, for example, signal peptide, amino acids 1 to 21; extracellular domain, amino acids 22 to 165; transmembrane domain, amino acids 166 to 186; intracellular domain, amino acids 187 to 287. It is understood that a “LAIR1 nucleic acid molecule” refers to a polynucleotide encoding a LAIR1 polypeptide.

| (NP_002278.2; SEQ ID NO: 39) | |
|---|---|
| 1 | MSPHPTALLG LVLCLAQTIH TQEEDLPRPS ISAEPGTVIP LGSHVTFVCR GPVGVQTFRL |
| 61 | ERDSRSTYND TEDVSQASPS ESEARFRIDS VREGNAGLYR CIYYKPPKWS EQSDYLELLV |
| 121 | KESSGGPDSP DTEPGSSAGP TQRPSDNSHN EHAPASQGLK AEHLYILIGV SVVFLFCLLL |
| 181 | LVLFCLHRQN QIKQGPPRSK DEEQKPQQRP DLAVDVLERT ADKATVNGLP EKDRETDTSA |
| 241 | LAAGSSQEVT YAQLDHWALT QRTARAVSPQ STKPMAESIT YAAVARH |

In one embodiment, the invention provides a LAIRI DN form. In one embodiment, the LAIR1 DN form comprises the extracellular ligand binding domain of LAIR1. In one embodiment, the LAIRI DN form comprises the extracellular ligand binding domain of LAIRI and a transmembrane domain (e.g., mature form). In another embodiment, the LAIRI DN form comprises the extracellular ligand binding domain of LAIR1, a transmembrane domain and a signal peptide (e.g., precursor form). The invention also provides encoding polypeptides and nucleic acids of the LAIR1 DN forms of the invention. In a particular embodiment, the LAIRI extracellular ligand binding domain is fused to one or more heterologous polypeptide sequences, that is, the LAIRI DN form is chimeric. For example, the LAIRI extracellular ligand binding domain can be fused at its N-terminus to a signal peptide that is optionally a heterologous signal peptide, including various signal peptides described herein. In addition, a LAIRI DN form can comprise a transmembrane domain that is optionally a heterologous transmembrane domain, including any of various transmembrane domains described herein.

In an embodiment of the invention, the LAIRI DN form can comprise the extracellular domain, or a ligand binding portion thereof, of LAIR1, for example, amino acids 22 to 165 corresponding to the extracellular domain of LAIR1 (GenBank NP_002278.2; SEQ ID NO:39). A cell expressing such a LAIR1 DN form should lack the ability or have reduced ability to signal in a LAIR1 immune checkpoint pathway. In one embodiment, a LAIR1 DN form is a deletion mutant having a deletion of the intracellular domain, for example, amino acids 187 to 287 of LAIR1 (GenBank NP_002278.2; SEQ ID NO:39), or a portion thereof, such that intracellular signaling of the immune checkpoint pathway mediated by LAIR1 is reduced or inhibited. [00172]2B4. Natural Killer Cell Receptor 2B4 (2B4) mediates non-MHC restricted cell killing on NK cells and subsets of T cells. The 2B4-S isoform is believed to be an activating receptor, and the 2B4—L isoform is believed to be a negative immune regulator of immune cells. 2B4 becomes engaged upon binding its high-affinity ligand, CD48. 2B4 contains a tyrosine-based switch motif, a molecular switch that allows the protein to associate with various phosphatases. 2B4 has also been designated CD244 (cluster of differentiation 244).

A 2B4 polypeptide can have an amino acid sequence corresponding to GenBank No. NP_001160135.1 (GI:262263435), sequence provided below, or fragments thereof. See GenBank NP_001160135.1 for reference to domains within 2B4, for example, signal peptide, amino acids 1 to 18; extracellular domain, amino acids 19 to 229; transmembrane domain, amino acids 230 to 250; intracellular domain, amino acids 251 to 370. It is understood that a “2B4 nucleic acid molecule” refers to a polynucleotide encoding a 2B4 polypeptide.

| (NP_001160135.1; SEQ ID NO: 40) | |
|---|---|
| 1 | MLGQVVTLIL LLLLKVYQGK GCQGSADHVV SISGVPLQLQ PNSIQTKVDS IAWKKLLPSQ |
| 61 | NGFHHILKWE NGSLPSNTSN DRFSFIVKNL SLLIKAAQQQ DSGLYCLEVT SISGKVQTAT |
| 121 | FQVFVFESLL PDKVEKPRLQ GQGKILDRGR CQVALSCLVS RDGNVSYAWY RGSKLIQTAG |
| 181 | NLTYLDEEVD INGTHTYTCN VSNPVSWESH TLNLTQDCQN AHQEFRFWPF LVIIVILSAL |
| 241 | FLGTLACFCV WRRKRKEKQS ETSPKEFLTI YEDVKDLKTR RNHEQEQTFP GGGSTIYSMI |
| 301 | QSQSSAPTSQ EPAYTLYSLI QPSRKSGSRK RNHSPSFNST IYEVIGKSQP KAQNPARLSR |
| 361 | KELENFDVYS |

In one embodiment, the invention provides a 2B4 DN form. In one embodiment, the 2B4 DN form comprises the extracellular ligand binding domain of 2B4. In one embodiment, the 2B4 DN form comprises the extracellular ligand binding domain of 2B4 and a transmembrane domain (e.g., mature form). In another embodiment, the 2B4 DN form comprises the extracellular ligand binding domain of 2B4, a transmembrane domain and a signal peptide (e.g., precursor form). The invention also provides encoding polypeptides and nucleic acids of the 2B4 DN forms of the invention. In a particular embodiment, the 2B4 extracellular ligand binding domain is fused to one or more heterologous polypeptide sequences, that is, the 2B4 DN form is chimeric. For example, the 2B4 extracellular ligand binding domain can be fused at its N-terminus to a signal peptide that is optionally a heterologous signal peptide, including various signal peptides described herein. In addition, a 2B4 DN form can comprise a transmembrane domain that is optionally a heterologous transmembrane domain, including any of various transmembrane domains described herein.

In an embodiment of the invention, the 2B4 DN form can comprise the extracellular domain, or a ligand binding portion thereof, of 2B4, for example, amino acids 19 to 229 corresponding to the extracellular domain of 2B4 (GenBank NP_001160135.1; SEQ ID NO:40). A cell expressing such a 2B4 DN form should lack the ability or have reduced ability to signal in a 2B4 immune checkpoint pathway. In one embodiment, a 2B4 DN form is a deletion mutant having a deletion of the intracellular domain, for example, amino acids 251 to 370 of 2B4 (GenBank NP_001160135.1; SEQ ID NO:40), or a portion thereof, such that intracellular signaling of the immune checkpoint pathway mediated by 2B4 is reduced or inhibited.

CD160. CD160 is a glycosylphosphatidylinositol-anchored molecule containing a single IgV-like domain that binds to HVEM and functions as a co-inhibitory receptor on T cells. A CD160 polypeptide can have an amino acid sequence corresponding to GenBank NP_008984.1 (GI:5901910), sequence provided below, or fragments thereof. See GenBank NP_008984.1 for reference to domains within CD160, for example, signal peptide, amino acids 1 to 26; extracellular domain, amino acids 27 to 159. It is understood that a “CD160 nucleic acid molecule” refers to a polynucleotide encoding a CD160 polypeptide.

| (NP_008984.1; SEQ ID NO: 41) | |
|---|---|
| 1 | MLLEPGRGCC ALAILLAIVD IQSGGCINIT SSASQEGTRL NLICTVWHKK EEAEGFVVFL |
| 61 | CKDRSGDCSP ETSLKQLRLK RDPGIDGVGE ISSQLMFTIS QVTPLHSGTY QCCARSQKSG |
| 121 | IRLQGHFFSI LFTETGNYTV TGLKQRQHLE FSHNEGTLSS GFLQEKVWVM LVTSLVALQA |
| 181 | L |

In one embodiment, the invention provides a CD160 DN form. In one embodiment, the CD160 DN form comprises the extracellular ligand binding domain of CD160. In one embodiment, the CD160 DN form comprises the extracellular ligand binding domain of CD160 and a transmembrane domain (e.g., mature form). In another embodiment, the CD160 DN form comprises the extracellular ligand binding domain of CD160, a transmembrane domain and a signal peptide (e.g., precursor form). The invention also provides encoding polypeptides and nucleic acids of the CD160 DN forms of the invention. In a particular embodiment, the CD1 60 extracellular ligand binding domain is fused to one or more heterologous polypeptide sequences, that is, the CD160 DN form is chimeric. For example, the CD160 extracellular ligand binding domain can be fused at its N-terminus to a signal peptide that is optionally a heterologous signal peptide, including various signal peptides described herein. In addition, a CD160 DN form can comprise a transmembrane domain that is a heterologous transmembrane domain, including any of various transmembrane domains described herein.

In an embodiment of the invention, the CD160 DN form can comprise the extracellular domain, or a ligand binding portion thereof, of CD160, for example, amino acids 27 to 159 corresponding to the extracellular domain of CD160 (GenBank NP_008984.1; SEQ ID NO:41). A cell expressing such a CD160 DN form should lack the ability or have reduced ability to signal in an immune checkpoint pathway. In one embodiment, the CD160 DN form comprises the extracellular domain of CD160, or a ligand binding portion thereof, and a transmembrane domain derived from a heterologous polypeptide, including but not limited to one of the transmembrane domains described herein. In one non-limiting embodiment, the CD160 DN form comprises the transmembrane domain of CD8. In a cell expressing the CD160 DN form, intracellular signaling of the immune checkpoint pathway mediated by CD160 should be reduced or inhibited.

TGF-β Receptor Type 2. TGF-β receptor type 2 binds to TGF-β and a type 1 receptor dimer forming a heterotetrameric complex with the ligand. A TGF-β receptor type 2 polypeptide can have an amino acid sequence corresponding to GenBank No. NP_001020018.1 (GI:67782326), sequence provided below, or fragments thereof. See GenBank NP_001020018.1 for reference to domains within TGF-β receptor type 2, for example, signal peptide, amino acids 1 to 22; extracellular domain, amino acids 23 to 191; transmembrane domain, amino acids 192 to 212; intracellular domain, amino acids 213 to 592 (see also annotation in UniProtKB—P37173). It is understood that a “TGF-β receptor type 2 nucleic acid molecule” refers to a polynucleotide encoding a TGF-β receptor type 2 polypeptide.

| (NP_001020018.1, SEQ ID NO: 42) | |
|---|---|
| 1 | MGRGLLRGLW PLHIVLWTRI ASTIPPHVQK SDVEMEAQKD EIICPSCNRT AHPLRHINND |
| 61 | MIVTDNNGAV KFPQLCKFCD VRFSTCDNQK SCMSNCSITS ICEKPQEVCV AVWRKNDENI |
| 121 | TLETVCHDPK LPYHDFILED AASPKCIMKE KKKPGETFFM CSCSSDECND NIIFSEEYNT |
| 181 | SNPDLLLVIF QVTGISLLPP LGVAISVIII FYCYRVNRQQ KLSSTWETGK TRKLMEFSEH |
| 241 | CAIILEDDRS DISSTCANNI NHNTELLPIE LDTLVGKGRF AEVYKAKLKQ NTSEQFETVA |
| 301 | VKIFPYEEYA SWKTEKDIFS DINLKHENIL QFLTAEERKT ELGKQYWLIT AFHAKGNLQE |
| 361 | YLTRHVISWE DLRKLGSSLA RGIAHLHSDH TPCGRPKMPI VHRDLKSSNI LVKNDLTCCL |
| 421 | CDFGLSLRLD PTLSVDDLAN SGQVGTARYM APEVLESRMN LENVESFKQT DVYSMALVLW |
| 481 | EMTSRCNAVG EVKDYEPPFG SKVREHPCVE SMKDNVLRDR GRPEIPSFWL NHQGIQMVCE |
| 541 | TLTECWDHDP EARLTAQCVA ERFSELEHLD RLSGRSCSEE KIPEDGSLNT TK |

In one embodiment, the invention provides a TGFβ receptor DN form. In one embodiment, the TGFβ receptor DN form comprises the extracellular ligand binding domain of TGFβ receptor. In one embodiment, the TGFβ receptor DN form comprises the extracellular ligand binding domain of TGFβ receptor and a transmembrane domain (e.g., mature form). In another embodiment, the TGFβ receptor DN form comprises the extracellular ligand binding domain of TGFβ receptor, a transmembrane domain and a signal peptide (e.g., precursor form). The invention also provides encoding polypeptides and nucleic acids of the TGF-β receptor DN forms of the invention. In a particular embodiment, the TGFβ receptor extracellular ligand binding domain is fused to one or more heterologous polypeptide sequences, that is, the TGFβ receptor DN form is chimeric. For example, the TGFβ receptor extracellular ligand binding domain can be fused at its N-terminus to a signal peptide that is optionally a heterologous signal peptide, including various signal peptides described herein. In addition, a TGFβ receptor DN form can comprise a transmembrane domain that is a heterologous transmembrane domain, including any of various transmembrane domains described herein.

TGFβ receptor DN forms have been described previously (see, for example, Bottinger et al., EMBO J. 16:2621-2633 (1997), describing a DN form comprising TGFβ receptor extracellular and transmembrane domains; Foster et al., J. Immunother. 31:500-505 (2008); Bollard et al., Blood 99:3179-3187 (2002); Wieser et al., Mol. Cell. Biol. 13:7239-7247 (1993)). In an embodiment of the invention, the TGFβ receptor DN form can comprise the extracellular domain, or a ligand binding portion thereof, of TGFβ receptor, for example, amino acids 23 to 191 corresponding to the extracellular domain of TGFβ receptor (GenBank NP_001020018.1, SEQ ID NO:42). A cell expressing such a TGFβ receptor DN form lacks the ability or has reduced ability to signal in the cell. In one embodiment, a TGFβ receptor DN form is a deletion mutant having a deletion of the intracellular domain, for example, amino acids 213 to 592 of TGFβ receptor (GenBank NP_001020018.1, SEQ ID NO:42), or a portion thereof, such that intracellular signaling of mediated by TGFβ receptor is reduced or inhibited (see also Bottinger et al., EMBO J. 16:2621-2633 (1997); Foster et al., J. Immunother. 31:500-505 (2008); Bollard et al., Blood 99:3179-3187 (2002); Wieser et al., Mol. Cell. Biol. 13:7239-7247 (1993)).

It is understood that, optionally, a second DN form of an inhibitor of a cell-mediated immune response, such as an immune checkpoint inhibitor, can be expressed in a cell of the invention. In this case, it can be desirable to inhibit more than one cell-mediated immune response in the same cell. Thus, a cell can express two or more DN forms, each directed to a different inhibitor of a cell-mediated immune response, including those described above. For example, a DN form of PD-1 can be co-expressed in a cell with a DN form of TGF-β receptor, a DN form of PD-1 can be co-expressed with a DN form of CTLA-4, a CTLA-4 DN form can be co-expressed with a DN form of TGF-β, and so forth, as desired, including combinations of any of the DN forms described above

In addition to immune cells or precursor cells thereof, the invention also provides a cell comprising a DN form polypeptide. The invention additionally provides a cell comprising a nucleic acid of the invention, which encodes a DN form polypeptide of the invention. Further provided is a cell comprising the vector of the invention. The cells of the invention can express a DN form of the invention, or an encoding nucleic acid.

Additionally provided are recombinant cells expressing polypeptides, nucleic acids and/or vectors of the invention. Such a recombinant cell can be an immune cell, such as a T cell, or a precursor cell thereof, that is used to express a cancer antigen CAR and/or a DN form of the invention. Such recombinant immune cells are described in more detail above. Recombinant cells can be used for genetic manipulations prior to transduction of the immune cells or precursor cells thereof to be used therapeutically, such as generating constructs of the polypeptides and encoding nucleic acids of the invention, and/or for generating nucleic acid material for incorporation into a vector for expression in an immune cell. Such cells can include, but are not limited to, bacterial cells, in particular Escherichia coli, yeast cells, such as Saccharomyces cerevisiae, Pichia pastoris, and the like. Such recombinant cells can be used to produce polypeptides and/or encoding nucleic acids of the invention encoding a DN form, which can be isolated or purified, if desired, from said cells using routine molecular biology and protein purification techniques.

### 6.4. Methods of Treatment

The invention also relates to methods of treating cancer using the cells of the invention. In one embodiment, the methods can include administering an immune cell, or precursor cell thereof, expressing a cancer antigen CAR and a DN form of an inhibitor of a cell-mediated immune response. The cancer antigen is chosen to target a cancer of the subject. In another embodiment, the methods can include administering a cancer-antigen specific immune cell, such as a T cell, or precursor cell thereof, where the cell recombinantly expresses a DN form of an inhibitor of a cell-mediated immune response.

The invention relates to various methods of using the immune cells, for example, T cells, or precursor cells thereof, expressing a DN form of an inhibitor of a cell-mediated immune response, or expressing a cancer antigen-specific CAR and a DN form of an inhibitor of a cell-mediated immune response. The cells are administered as a population of cells expressing a DN form or expressing a cancer antigen-specific CAR and a DN form. Optionally, the cells to be administered can be purified or enriched for the cells of the invention. For example, the methods of the invention can be used to treat cancer or reduce tumor burden in a subject. In one embodiment, the methods of the invention are used to treat cancer. It is understood that a method of treating cancer can include any effect that ameliorates a sign or symptom associated with cancer. Such signs or symptoms include, but are not limited to, reducing tumor burden, including inhibiting growth of a tumor, slowing the growth rate of a tumor, reducing the size of a tumor, reducing the number of tumors, eliminating a tumor, all of which can be measured using routine tumor imaging techniques well known in the art. Other signs or symptoms associated with cancer include, but are not limited to, fatigue, pain, weight loss, and other signs or symptoms associated with various cancers. In one non-limiting example, the methods of the invention can reduce tumor burden. Thus, administration of the cells of the invention can reduce the number of tumor cells, reduce tumor size, and/or eradicate the tumor in the subject. The tumor can be a solid tumor. Non-limiting examples of a solid tumor include mesothelioma, lung cancer, pancreatic cancer, ovarian cancer, breast cancer, colon cancer, pleural tumor, glioblastoma, esophageal cancer, gastric cancer, and synovial sarcoma. The methods of the invention can also provide for increased or lengthened survival of a subject having cancer. Additionally, methods of the invention can provide for an increased immune response in the subject against the cancer.

In the methods of the invention, the immune cells or precursor cells thereof are administered to a subject in need of cancer treatment. The subject can be a mammal, in particular a human. Preferably, the subject is a human. Suitable human subjects for therapy include those with “advanced disease” or “high tumor burden” who bear a clinically measurable tumor. A clinically measurable tumor is one that can be detected on the basis of tumor mass, for example, by palpation, CAT scan, sonogram, mammogram, X-ray, and the like. Positive biochemical or histopathologic markers can also be used to identify this population. A pharmaceutical composition comprising a cell of the invention is administered to a subject to elicit an anti-cancer response, with the objective of palliating the subject's condition. Reduction in tumor mass of a subject having a tumor can occur, but any clinical improvement constitutes a benefit. Clinical improvement comprises decreased risk or rate of progression or reduction in pathological consequences of the tumor.

Another group of suitable subjects can be a subject who has a history of cancer, but has been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for different types of cancers. Features typical of high-risk subgroups are those in which the tumor has invaded neighboring tissues, or who show involvement of lymph nodes. Optionally, a cell of the invention can be administered for treatment prophylactically to prevent the occurrence of cancer in a subject suspected of having a predisposition to a cancer, for example, based on family history and/or genetic testing.

The subject can have an advanced form of disease, in which case the treatment objective can include mitigation or reversal of disease progression, and/or amelioration of side effects. The subjects can have a history of the condition, for which they have already been treated, in which case the therapeutic objective can be to decrease or delay the risk of recurrence. Additionally, refractory or recurrent malignancies can be treated using the cells of the invention.

The cells of the invention are administered to a subject, such as a human subject, in need of cancer treatment. The cancer can involve a solid tumor or a blood cancer not involving a solid tumor. Cancers to be treated using the cells of the invention comprise cancers typically responsive to immunotherapy. Exemplary types of cancers include, but are not limited to, carcinomas, sarcoma, leukemia, lymphoma, multiple myeloma, melanoma, brain and spinal cord tumors, germ cell tumors, neuroendocrine tumors, carcinoid tumors, and the like. The cancer can be a solid tumor or a blood cancer that does not form a solid tumor. In the case of a solid tumor, the tumor can be a primary tumor or a metastatic tumor.

Examples of other neoplasias or cancers that can be treated using the methods of the invention include bone cancer, intestinal cancer, liver cancer, skin cancer, cancer of the head or neck, melanoma (cutaneous or intraocular malignant melanoma), renal cancer (for example, clear cell carcinoma), throat cancer, prostate cancer (for example, hormone refractory prostate adenocarcinoma), blood cancers (for example, leukemias, lymphomas, and myelomas), uterine cancer, rectal cancer, cancer of the anal region, bladder cancer, brain cancer, stomach cancer, testicular cancer, carcinoma of the fallopian tubes, carcinoma of the endometrium, carcinoma of the cervix, carcinoma of the vagina, carcinoma of the vulva, leukemias (for example, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease, Waldenstrom's macroglobulinemia), cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, heavy chain disease, and solid tumors such as sarcomas and carcinomas, for example, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilm's tumor, cervical cancer, uterine cancer, testicular cancer, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma.

In one embodiment, the methods of the invention are used to treat a cancer selected from malignant pleural disease, mesothelioma, lung cancer (for example, non-small cell lung cancer), pancreatic cancer, ovarian cancer, breast cancer (for example, metastatic breast cancer, metastatic triple-negative breast cancer), colon cancer, pleural tumor, glioblastoma, esophageal cancer, gastric cancer, and synovial sarcoma. The invention provides therapies that are particularly useful for treating solid tumors, for example, malignant pleural disease, mesothelioma, lung cancer, pancreatic cancer, ovarian cancer, breast cancer, colon cancer, pleural tumor, glioblastoma, esophageal cancer, gastric cancer, and synovial sarcoma. Solid tumors can be primary tumors or tumors in a metastatic state. In the case of a mesothelin directed CAR, mesothelin expressing tumors, include, for example, breast cancer, lung cancer, ovarian cancer, pancreatic cancer, esophagus cancer, colon cancer, gastric cancer, and malignant pleural mesothelioma (MPM).

In a specific embodiment, the cells recombinantly expressing a CAR and DN form that are administered to the subject comprise both CD4⁺ and CD8⁺ T cells, with the aim of generating both helper and cytotoxic T lymphocyte (CTL) responses in the subject.

For treatment, the amount administered is an amount effective for producing the desired effect. An effective amount or therapeutically effective amount is an amount sufficient to provide a beneficial or desired clinical result upon treatment. An effective amount can be provided in a single administration or a series of administrations (one or more doses). An effective amount can be provided in a bolus or by continuous perfusion. In terms of treatment, an effective amount is an amount that is sufficient to palliate, ameliorate, stabilize, reverse or slow the progression of the disease, or otherwise reduce the pathological consequences of the disease. The effective amount can be determined by the physician for a particular subject. Several factors are typically taken into account when determining an appropriate dosage to achieve an effective amount. These factors include age, sex and weight of the subject, the condition being treated, the severity of the condition and the form and effective concentration of the cells of the invention being administered.

The cells of the invention are generally administered as a dose based on cells per kilogram (cells/kg) of body weight of the subject to which the cells are administered. Generally the cell doses are in the range of about 10⁴ to about 10¹⁰ cells/kg of body weight, for example, about 10⁵ to about 10⁹, about 10⁵ to about 10⁸, about 10⁵ to about 107, or about 105 to 10⁶, depending on the mode and location of administration. In general, in the case of systemic administration, a higher dose is used than in regional administration, where the immune cells of the invention are administered in the region of a tumor. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 1×10⁸, 6×10⁴, to 1×10⁸, 7×10⁴ to 1×10⁸, 8×10⁴ to 1×10⁸, 9×10⁴ to 1×10⁸, 1×10⁵ to 1×10⁸, for example, 1×10⁵ to 9×10⁷, 1×10⁵ to 8×10⁷, 1×10⁵ to 7×10⁷, 1×10⁵ to 6×10⁷, 1×10⁵ to 5×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 3×10⁷, 2×10⁵ to 2×10⁷, 2×10⁵ to 1×10⁷, 2×10⁵ to 9×106 2×10⁵ to 8×10⁶, 2×10⁵ to 7×106 2×10⁵ to 6×10⁶, 2×10⁵ to 5×10⁶, 2×10⁵ to 4×10⁶, 3×10⁵ to 3×10⁶ cells/kg, and the like. Such dose ranges can be particularly useful for regional administration. In a particular embodiment, cells are provided in a dose of 1×10⁵ to 1×10⁸, for example 1×10⁵ to 1×10⁷, 1×10⁵ to 1×10⁶, 1×10⁶ to 1×10⁸, 1×10⁶ to 1×10⁷, 1×10⁷ to 1×10, 1×10⁵ to 5×10⁶, in particular 1×10⁵ to 3×10⁶ or 3×10⁵ to 3×10⁶ cells/kg for regional administration, for example, intrapleural administration. Exemplary dose ranges also can include, but are not limited to, 5×10⁵ to 1×10⁸, for example, 6×10⁵ to 1×10⁸, 7×10⁵ to 1×10⁸, 8×10⁵ to 1×10⁸, 9×10⁵ to 1×10⁸, 1×10⁶ to 1×10⁸, 1×10⁶ to 9×10⁷, 1×10⁶ to 8×10⁷, 1×10⁶ to 7×10⁷, 1×10⁶ to 6×10⁷, 1×10⁶ to 5×10⁷, 1×10⁶ to 4×10⁷, 1×10⁶ to 3×10⁷ cells/kg, and the like. Such does can be particularly useful for systemic administration. In a particular embodiment, cells are provided in a dose of 1×10⁶ to 3×10⁷ cells/kg for systemic administration. Exemplary cell doses include, but are not limited to, a dose of 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹ and so forth in the range of about 10⁴ to about 10¹⁰ cells/kg. In addition, the dose can also be adjusted to account for whether a single dose is being administered or whether multiple doses are being administered. The precise determination of what would be considered an effective dose can be based on factors individual to each subject, including their size, age, sex, weight, and condition of the particular subject, as described above. Dosages can be readily determined by those skilled in the art based on the disclosure herein and knowledge in the art.

The cells of the invention can be administered by any methods known in the art, including, but not limited to, pleural administration, intravenous administration, subcutaneous administration, intranodal administration, intratumoral administration, intrathecal administration, intrapleural administration, intraperitoneal administration, intracranial administration, and direct administration to the thymus. In one embodiment, the cells of the invention can be delivered regionally to a tumor using well known methods, including but not limited to, hepatic or aortic pump; limb, lung or liver perfusion; in the portal vein; through a venous shunt; in a cavity or in a vein that is nearby a tumor, and the like. In another embodiment, the cells of the invention can be administered systemically. In a preferred embodiment, the cells are administered regionally at the site of a tumor. The cells can also be administered intratumorally, for example, by direct injection of the cells at the site of a tumor and/or into the tumor vasculature. For example, in the case of malignant pleural disease, mesothelioma or lung cancer, administration is preferably by intrapleural administration (see Adusumilli et al., Science Translational Medicine 6(261):261ra1 51 (2014)). One skilled in the art can select a suitable mode of administration based on the type of cancer and/or location of a tumor to be treated. The cells can be introduced by injection or catheter. In one embodiment, the cells are pleurally administered to the subject in need, for example, using an intrapleural catheter. Optionally, expansion and/or differentiation agents can be administered to the subject prior to, during or after administration of cells to increase production of the cells of the invention in vivo.

Proliferation of the cells of the invention is generally done ex vivo, prior to administration to a subject, and can be desirable in vivo after administration to a subject (see Kaiser et al., Cancer Gene Therapy 22:72-78 (2015)). Cell proliferation should be accompanied by cell survival to permit cell expansion and persistence, such as with T cells.

The methods of the invention can further comprise adjuvant therapy in combination with, either prior to, during, or after treatment with the cells of the invention. Thus, the cell therapy methods of the invention can be used with other standard cancer care and/or therapies that are compatible with administration of the cells of the invention.

The methods of the invention relate to generating cancer-targeted immune cells, or precursor cells thereof, for adoptive therapy to enhance immune cell function through the design of improved antigen receptors and inclusion of cell intrinsic inhibition of immune checkpoint pathways, such as with co-expression of DN forms of an inhibitor of a cell-mediated immune response. Optionally, the methods of administering cells of the invention can additionally include immunomodulation of the host to facilitate the effectiveness of the administered cells of the invention in combination therapy. In an embodiment of the invention, the methods of the invention can further comprise administering at least one immunomodulatory agent. Non-limiting examples of immunomodulatory agents include immunostimulatory agents, checkpoint immune blockade agents, radiation therapy agents, and chemotherapy agents. In certain embodiments, the immunomodulatory agent is an immunostimulatory agent. In one embodiment, the immunostimulatory agent is a cytokine, including but not limited to, IL-2, IL-3, IL-6, IL-7, IL-11, IL-12, IL-15, IL-17, and IL-21. Other exemplary immunostimulatory agents include, but are not limited to, colony stimulating factors, such as G-, M- and GM-CSF, interferons, for example, γ-interferon, and the like. In one embodiment, the methods of the invention further comprise administering IL-2 or GM-CSF to the subject. In a specific embodiment, IL-2 is administered to the subject. The IL-2 or GM-CSF can be administered before, during or after cell therapy using cells of the invention (i.e., concurrently or sequentially), as desired. In a specific embodiment the cytokine (e.g., IL-2 or GM-CSF) is administered on the same day, or during the same week, or within 2 weeks, of the cell therapy using cells of the invention. In a particular embodiment, IL-2 is administered in a dose of about 50,000 to 800,000 international units (IU) per kilogram of body weight, for example, about 50,000 to 720,000, 50,000 to 500,000, 50,000 to 250,000, 50,000 to 200,000, 50,000 to 150,000, 50,000 to 100,000, or about 720,000 IU/kg (Robbins et al., J. C/in. Oncol. 29:917-924 (2011)). In a non-limiting embodiment, IL-2 is administered in a dose of about 50,000, 55,000, 60,000, 61,000, 62,000, 63,000, 64,000, 65,000, 66,000, 67,000, 68,000, 69,000, 70,000, 71,000, 72,000, 73,000, 74,000, 75,000, 76,000, 77,000, 78,000, 79,000, 80,000, 85,000, 90,000, 95,000, 100,000, 110,000, 120,000, 130,000, 140,000, 150,000, 160,000, 170,000, 180,000, 190,000, 200,000, 210,000, 220,000, 230,000, 240,000, 250,000, 260,000, 270,000, 280,000, 290,000, 300,000, 320,000, 340,000, 360,000, 380,000, 400,000, 420,000, 440,000, 460,000, 480,000, 500,000, 520,000, 540,000, 560,000, 580,000, 600,000, 620,000, 640,000, 660,000, 680,000, 700,000, 720,000, 740,000, 760,000, 780,000 or 800,000 IU/kg. Given the improved efficacy of immune cell therapy using cells of the invention expressing a CAR and DN form, it is expected that the doses of cytokines, such as IL-2, suitable as combination therapy with a cell of the invention can be lower than that used with other therapies using cytokines. Administering a cytokine, for example, IL-2 or GM-CSF, is particularly useful if the CAR expressed in the immune cell results in reduced expression of an immune cell stimulatory cytokine, such as IL-2 or GM-CSF. The cytokine can be administered to enhance the efficacy of the immune cells of the invention expressing the CAR and DN form. As described in the Example hereinafter, T cells expressing a PD-1 DN form and MBBz CAR, having 4-1BB as a co-stimulatory signaling domain, exhibit decreased expression of IL-2, whereas T cells expressing a PD-1 DN form and M28z CAR, having CD28 as a co-stimulatory signaling domain, have increased expression of IL-2 (see Example). Accordingly, the invention provides for treating cancer in a subject having cancer by administering to the subject T cells expressing a PD-1 DN form and a MBBz CAR, which CAR has 4-1BB as a co-stimulatory signaling domain, and administering to the subject IL-2. A person skilled in the art can readily assay an immune cell of the invention for expression of immunostimulatory cytokines and, if desired, optionally administer an immunostimulatory cytokine that is deficiently expressed by the cells to a subject being treated with the cells. Such a combination therapy including an immunostimulatory cytokine can be used to increase the efficacy of immune cell therapy using such cells, for example, cells expressing a DN form of an immune checkpoint inhibitor with reduced immunostimulatory cytokine production.

Additional immunostimulatory agents include agonist costimulatory monoclonal antibodies, such as anti-4-1BB antibodies, anti-OX40 antibodies, and anti-ICOS antibodies. In one embodiment, the agonist costimulatory monoclonal antibody is an anti-4-1BB antibody.

Among all immunotherapeutic approaches, IL-12, a multifunctional cytokine, has been considered to be one of the most promising approaches to treat breast cancer (Boggio et al., Cancer Res. 60:359-364 (2000); Czerniecki et al., Cancer Res. 67:1842-1852 (2007); Nanni et al., J Exp. Med. 194:1195-1205 (2001)). IL-12 is considered a master regulator of adaptive type 1 cell-mediated immunity, the critical pathway involved in antitumor responses (Del Vecchio et al., Clin. Cancer Res. 13:4677-4685 (2007)). IL-12 modulates antitumor responses at various levels, including polarization of CD4 T cells toward a Th1 phenotype (Wesa et al., J. Immunother. 30, 75-82 (2007)), boosting of T cell and NK effector functions (Curtsinger et al., J. Exp. Med 197:1141-1151 (2003)), remodeling the innate immune response (Chmielewski et al., Cancer Res. 71:5697-5706 (2011)), and regulating tumor angiogenesis (Voest et al., J. Natl. Cancer. Inst. 87:581-586 (1995)). Among 148 clinical trials including administration of IL-12 to patients with cancer, successful phase II studies with intraperitoneal (Lenzi et al., Clin. Cancer Res. 8:3686-3695 (2002); Lenzi et al., J. Transl. Med. 5:66 (2007)) or subcutaneous (Mahvi et al., Cancer Gene Ther. 14:717-723 (2007); Kang et al., Hum. Gene Ther. 12:671-684 (2001)) IL-12 have shown that paracrine secretion of IL-12, generated by gene transfer, can induce immunity against the tumor locally and at a distant site. Although several studies have documented the anticancer effectiveness of IL-12 in preclinical models of breast cancer (Boggio et al., Cancer Res. 60:359-364 (2000); Nanni et al., J. Exp. Med. 194:1195-1205 (2001); Brunda et al., J Exp. Med. 178:1223-1230 (1993)), the significant toxicity resulting from administration of recombinant human IL-12 observed in several clinical trials in advanced cancers precludes its clinical use. To overcome this limitation, a number of groups have demonstrated that intratumoral delivery of IL-12, using adenoviral vectors, induces tumor regression and T cell activation in preclinical models of breast cancer (Gyorffy et al., J. Immunol. 166:6212-6217 (2001); Bramson et al., Hum. Gene Ther. 7:1995-2002 (1996)). More recently, polylactic acid microspheres were used to release IL-12 into the tumor, and it was found that the antitumor response was mediated primarily by NK cells (Sabel et al., Breast Cancer Res. Treat. 122:325-336 (2010)). Others have used mesenchymal stromal cells to locally deliver IL-12 to mouse breast cancer (Eliopoulos et al., Cancer Res. 68, 4810-4818 (2008)). A phase I trial of paclitaxel and trastuzumab, in combination with IL-12, in patients with HER2/neu-expressing malignancies showed an impressive synergy between IL-12 and trastuzumab for stimulation of NK-cell cytokine secretion (Bekaii-Saab et al., MoL. Cancer Ther. 8:2983-2991 (2009)). Therefore, IL-12 is particularly useful as an anticancer agent to be used as a co-stimulant in an adoptive immune cell therapy approach, including the methods of the invention disclosed herein. The immunomodulating and antiangiogenic functions of IL-12 support the use of this cytokine in combination with a cell of the invention for treating cancers.

In another embodiment, the immunomodulatory agent is a co-stimulatory ligand. Co-stimulatory ligands include, without limitation, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L/CD154, CD137L/4-1BBL, TNF-α, CD134L/OX40L/CD252, CD27L/CD70, Fas ligand (FasL), CD30L/CD153, tumor necrosis factor beta (TNF$)/lymphotoxin-alpha (LTa), lymphotoxin-beta (LT$), CD257/B cell-activating factor (BAFF)/Blys/THANK/Tall-1, glucocorticoid-induced TNF Receptor ligand (GITRL), TNF-related apoptosis-inducing ligand (TRAIL), and LIGHT (TNFSF14). The immunoglobulin (Ig) superfamily is a large group of cell surface and soluble proteins that are involved in the recognition, binding, or adhesion processes of cells. These proteins share structural features with immunoglobulins, that is, they possess an immunoglobulin domain (fold). Immunoglobulin superfamily ligands include, without limitation, CD80 and CD86, both ligands for CD28. In some embodiments, the at least one co-stimulatory ligand is selected from the group consisting of 4-1BBL, CD80, CD86, CD70, OX40L, CD48, TNFRSF14, and the like.

In another embodiment, the immunomodulatory agent can be an immune checkpoint blockade agent. The administration of an immune checkpoint blockade agent supplements the inhibition of immune checkpoint blockade provided by expressing a DN form of an immune checkpoint inhibitor in a cell of the invention. Non-limiting examples of immune checkpoint blockade agents include anti-PD-L1 antibodies, anti-CTLA-4 antibodies, anti-PD-1 antibodies, anti-LAG3 antibodies, anti-B7-H3 antibodies, anti-TIM3 antibodies, and the like. Such immune checkpoint blockade agents include, but are not limited to, antibodies to PD-1, CTLA-4, BTLA, TIM-3, LAG-3, CD160, TIGIT, LAIR1, 2B4, and the like, or antibodies to the corresponding ligands for these receptors including, for example, PD-L1 (for PD-1); PD-L2 (for PD-1); CD80, CD86 (for CTLA-4); HVEM (for BTLA); Galectin-9, HMGB1 (for TIM-3); MHC II (for LAG-3); HVEM (for CD160); CD155, CD112, CD113 (for TIGIT); C1q, collagen (for LAIRI); CD48 (for 2B4), and the like. In one embodiment, the checkpoint immune blockade agent is an anti-PD-L1 antibody. It is understood that an antibody that inhibits the activity of an immune checkpoint inhibitor by binding to the immune checkpoint inhibitor receptor or its corresponding ligand, including receptors and ligands as disclosed herein, can be used as an immunomodulatory agent to further suppress the immunoinhibitory effect in an immune cell of the invention expressing a DN form. In a particular embodiment, the antibody will be to the immune checkpoint inhibitor, or its ligand, that corresponds to the DN form being expressed in the immune cell of the invention, which can be useful to further suppress any residual activity in the immune cell expressing the DN form. In certain embodiments, the methods of the invention can optionally include administration of an immune checkpoint blockade agent such as antibodies directed to the ligand and/or receptor of an immune checkpoint pathway.

In some embodiments, the immunomodulatory agent can be a radiation therapy agent. The localized, radiation-induced immunological milieu can provide the preconditions to enhance the engraftment of cells of the invention at the site of the tumor, thereby eliminating the need for systemic lymphodepleting regimens. The immunological responses resulting from a combination of radiation therapy, particularly low dose radiation therapy, and cell therapy methods of the invention also can enhance abscopal antitumor efficacy. In some embodiments, the immunomodulatory agent is a chemotherapy agent, including, but not limited to, cisplatin, cyclophosphamide, and the like. Cisplatin-induced secretion of chemokines and cytokines can promote cancer antigen-targeted cells of the invention and endogenous immune cell responses such as T-cell responses. Cyclophosphamide can function as a lymphodepleting agent, for example, as a preparatory lymphodepleting agent.

Tumor irradiation- and cisplatin therapy-induced tumoral and abscopal immunomodulation can provide the preconditioning required for better engraftment of cells f the invention. Co-stimulatory strategies, as described above, can potentiate the antitumor efficacy of both endogenous T cells and the cells of the invention.

In another embodiment, an immunomodulatory agent can be a “switch receptor.” The methods of the invention can additionally include administering immune cells expressing a CAR and a “switch receptor.” The switch receptor comprises at least a ligand binding domain of the extracellular region of an immune checkpoint inhibitor, fused to a transmembrane domain, fused to a cytoplasmic signaling domain (i.e., co-stimulatory domain) of an immunostimulatory molecule, thereby switching the activity upon ligand binding from immunoinhibitory to immunostimulatory (see e.g., Liu et al., Cancer Res. 76:1578-1 590 (2016)). In one embodiment, the immune checkpoint inhibitor extracellular domain is derived from an immune checkpoint inhibitor including, but not limited to, programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRI), natural killer cell receptor 2B4 (2B4), and CD160. The ligand binding domains that can be used for generation of such a switch receptor include those ligand binding domains described above for generating a DN form of an immune checkpoint inhibitor. In the case of a switch receptor, a cytoplasmic signaling domain (i.e., co-stimulatory domain) is fused to the extracellular ligand binding domain of the immune checkpoint inhibitor via a transmembrane domain. A cytoplasmic signaling domain that is a co-stimulatory domain can be derived, for example, from a receptor such as the co-stimulatory molecules described herein for use in a CAR, including but not limited to a 4-1BB polypeptide, a CD28 polypeptide, an OX40 polypeptide, an ICOS polypeptide, a DAP10 polypeptide, and a 2B4 polypeptide. A switch receptor also includes a transmembrane domain, which can be derived from the polypeptide from which the co-stimulatory domain is derived, from the polypeptide from which the extracellular ligand binding domain of the immune checkpoint inhibitor is derived, or it can be a transmembrane domain from another polypeptide, similar to the description herein of the transmembrane domains that can be utilized to generate a CAR or DN form.

The invention provides for recombinant expression by an immune cell of both a CAR and a switch receptor, which switch receptor comprises (i) at least the extracellular ligand binding domain of an immune checkpoint inhibitor, (ii) a transmembrane domain, and (iii) a co-stimulatory signaling domain. In a particular embodiment, the co-stimulatory signaling domain of the switch receptor is the intracellular signaling domain of 4-1BB. In another particular embodiment of the invention, the immune cell expressing the switch receptor expresses a CAR, where the co-stimulatory signaling domain of the CAR is the intracellular signaling domain of CD28. In another particular embodiment, the invention provides an immune cell expressing a switch receptor and a CAR, where the co-stimulatory signaling domain of the switch receptor is the intracellular signaling domain of 4-1BB and the co-stimulatory signaling domain of the CAR is the intracellular signaling domain of CD28.

In a method utilizing a switch receptor, the switch receptor can be transduced into the same cell in which the CAR and DN form are transduced, so that the cell recombinantly expresses all three constructs. Alternatively and preferably, the switch receptor is transduced into a cell in which the CAR, but not DN form is transduced, so as to produce a cell expressing both the switch receptor and CAR, which can be used in combination therapy with cells that express both the CAR and DN form but not the switch receptor. In this case, both types of cells, cells expressing a CAR and DN form, and cells expressing a CAR and a switch receptor, are administered to the subject. Generally, the two types of cells are administered concurrently, but can also be administered sequentially, for example, within 1 or 2 hours, or within 1 or 2 days, or on the same day, as each other, as desired. In a particular embodiment, the co-stimulatory signaling domain of the CAR is different than the co-stimulatory signaling domain of the switch receptor being expressed in the same cell. This should result in two co-stimulatory signaling domains in the same cell and enhanced efficacy of the cells for immune cell therapy. In the case where it is believed that the administered immune cells will proliferate sufficiently in the subject being treated such that additional doses of cells need not be administered, it may be suitable to administer the immune cells of the invention at the initiation of immune cell therapy. Optionally, the immune cells of the invention, including optionally immune cells that express a switch receptor, can be administered more than once, as needed.

Optionally, a cell of the invention can express a co-stimulatory receptor (CCR) that binds to an antigen different than the cancer antigen of the target cancer (see Sadelain, et al., Cancer Discovery 3(4):388-398 (2013), Chicaybam, et al., Int. Rev. Immunol. 30(5-6):294-311 (2011), Brentjens et al., Nature Medicine 9:279-286 (2003); U.S. Pat. No. 7,446,190 and U.S. 2013/0071414 (CD19-targeted CARs); Ahmed, et al., Clin. Cancer Res. 16(2):474-485(2010)(HER2-targeted CARs); Chekmasova, et al., Clin. Cancer Res. 16(14):3594-606 (2010)(MUC16-targeted CARs); Zhong, et al., Molecular Therapy, 18(2):413-420 (2010) and U.S. Pat. No. 7,446,190 (prostate-specific membrane antigen (PSMA)-targeted CARs), all of which are herein incorporated by reference. CCRs mimic co-stimulatory signals but, unlike CARs, do not provide a T cell activation signal (see Sadelain, et al., Cancer Discovery 3(4):388-398 (2013)). Immune cells expressing two or more antigen recognizing receptors are described in WO 2014/055668, which is herein incorporated by reference.

Administering an immunomodulatory agent in a combination therapy with an immune cell of the invention can occur concurrently with administration of the immune cells of the invention, for example, when immune cell therapy is initiated, or can occur sequentially at any time during the immune cell therapy, as desired. A person skilled in the art can readily determine appropriate regimens for administering cells of the invention and an immunomodulatory agent in a combination therapy, including the timing and dosing of an immunomodulatory agent to be used in a combination therapy, based on the needs of the subject being treated.

### 6.5. Pharmaceutical Compositions

The invention additionally provides pharmaceutical compositions comprising the cells of the invention. The pharmaceutical composition comprises an effective amount of a cell of the invention and a pharmaceutically acceptable carrier. The cells of the invention and compositions comprising the cells can be conveniently provided in sterile liquid preparations, for example, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

Sterile injectable solutions can be prepared by incorporating cells of the invention in a suitable amount of the appropriate solvent with various amounts of the other ingredients, as desired. Such compositions can include a pharmaceutically acceptable carrier, diluent, or excipient such as sterile water, physiological saline, glucose, dextrose, or the like, that are suitable for use with a cell composition and for administration to a subject such as a human. Suitable buffers for providing a cell composition are well known in the art. Any vehicle, diluent, or additive used is compatible with preserving the integrity and viability of the cells of the invention.

The compositions will generally be isotonic, that is, they have the same osmotic pressure as blood and lacrimal fluid. The desired isotonicity of the cell compositions of the invention can be accomplished using sodium chloride, or other pharmaceutically acceptable agents such as dextrose, boric acid, sodium tartrate, or other inorganic or organic solutes. Sodium chloride is preferred particularly for buffers containing sodium ions. One particularly useful buffer is saline, for example, normal saline. Those skilled in the art will recognize that the components of the compositions should be selected to be chemically inert and will not affect the viability or efficacy of the cells of the invention and will be compatible for administration to a subject, such as a human. The skilled artisan can readily determine the amount of cells and optional additives, vehicles, and/or carrier in compositions to be administered in methods of the invention.

The cells of the invention can be administered in any physiologically acceptable vehicle. Suitable doses for administration are described herein. A cell population comprising cells of the invention can comprise a purified population of cells. Those skilled in the art can readily determine the percentage of cells in a cell population using various well-known methods, as described herein. The ranges of purity in cell populations comprising genetically modified cells of the invention can be from about 50% to about 55%, from about 55% to about 60%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art; for example, a decrease in purity may require an increase in dosage.

The invention also provides kits for preparation of cells of the invention. In one embodiment, the kit comprises one or more vectors for generating a genetically engineered immune cell, such as a T cell, or precursor cell thereof, that expresses a DN form or co-expresses a cancer antigen CAR and DN form of an inhibitor of a cell-mediated immune response. The kits can be used to generate genetically engineered immune cells from autologous cells derived from a subject or from non-autologous cells to be administered to a compatible subject. In another embodiment, the kits can comprise cells of the invention, for example, autologous or non-autologous cells, for administration to a subject. In specific embodiments, the kits comprise the immune cells of the invention in one or more containers.

It is understood that modifications which do not substantially affect the activity of the various embodiments of this invention are also provided within the definition of the invention provided herein. Accordingly, the following example is intended to illustrate but not limit the present invention.

### 7. Example

### 7.1. Overview of Experiments

Following immune attack, solid tumors upregulate coinhibitory ligands that bind to and inhibit T cells. This adaptive resistance poses a hurdle for the treatment of solid tumors by chimeric antigen receptor (CAR) T-cell therapy, a promising treatment that has demonstrated complete remissions in patients with acute leukemia. As described below, it was investigated whether PD-1-mediated T-cell exhaustion could affect mesothelin-targeted CAR T cells in a mesothelioma model and whether cell-intrinsic strategies could be utilized to overcome checkpoint blockade. Using a clinically relevant, orthotopic mouse model of pleural mesothelioma, it was demonstrated that T cells expressing CD28 or 4-1BB-based second generation CARs, although persistent, are functionally inhibited within the tumor microenvironment (see below). While CD28 and 4-1BB CARs conferred similar proliferation and persistence of CAR T cells, the latter more durably retained their cytotoxic and cytokine secretion functions, resulting in improved survival in mice given low T-cell doses. CAR T cells that additionally expressed a PD-1 dominant negative receptor demonstrated functional persistence, induced superior tumor elimination and prolonged mouse survival. The results disclosed herein provide insights into CAR T-cell exhaustion in solid tumors and provide a strategy for combining CAR therapy with immune checkpoint blockade, for example, PD-1/PD-L1 blockade or other molecules involved in checkpoint blockade (see below).

The studies described below characterized the mechanisms of tumor-mediated T-cell inhibition in order to enhance the efficacy of T-cell immunotherapy for solid malignancies. As described below, MSLN-targeted CARs were designed that, when transduced into human T cells, provided tumor antigen recognition and antigen-specific effector function activation. Signaling domains were also designed that provide costimulatory signaling and/or coinhibitory blockade. In vitro, cytotoxicity, cytokine secretion, and T-cell proliferation were analyzed. In vivo experiments were performed to analyze strategies for optimizing T-cell therapy using clinically relevant mouse models of orthotopic MPM and metastatic lung cancer. Human cancer cells and human T cells were used to validate and facilitate the translation of M28z CAR to the clinic, as previously demonstrated for CD19 (Brentjens et al., Nat. Med. 9(3):279-286 (2003)) and PSMA (Gade et al., Cancer Res. 65(19):9080-9088 (2005)) CAR T cells.

As described below in more detail, low-level tumor infiltration was modeled, and it was found that CAR T cells can be susceptible to tumor cell-mediated immune-inhibition, resulting in impaired T-cell function and diminished tumor rejection. T cells engineered to resist PD-1 signaling displayed enhanced anti-tumor potency. As described below, following a single low-dose CAR T-cell therapy of advanced tumors, it was observed that, in response to CAR T-cell secreted cytokines, tumor cells upregulate PD-L1 leading to CAR T-cell inhibition and tumor relapse. To directly overcome the PD-L1-mediated immunosuppression, a PD-1 dominant negative receptor (PD-1 DNR) lacking the intracellular inhibitory signaling domain was designed. The cotransduction of PD-1 DNR with a CAR enhanced CAR T-cell function, resulting in a long-term cancer free survival following a single low-dose of CAR T cells. There is no previous disclosure of co-expressing a cancer antigen CAR with an immune checkpoint pathway receptor dominant negative, as is disclosed herein. The coexpression of an immune checkpoint pathway receptor DNR with a cancer antigen CAR is immediately translatable to the clinic since a DNR can be added to any CAR without inhibiting CAR function or adding toxicity. Without being bound by a particular theory, it is believed that the DNR simply binds (consumes) negative signal induced by its corresponding ligand (for example, PD-L1 in the case of PD-1) and avoids downstream signaling.

As described below, the presence and kinetics of tumor-mediated inhibition of CAR T cells were determined. By performing a comprehensive serial analysis of T-cell effector functions, it was established that even costimulated CAR T cells currently in clinical trials are subject to inhibition of their cytolytic and cytokine secretion functions upon repeated antigen encounter in vivo. The differing abilities of alternative costimulatory strategies (4-1BB vs. CD28) to withstand immuno inhibition was determined, as well as one of the mechanisms of tolerance (that is, PD-1 receptor/PD-1 ligand engagement). As further disclosed herein, it was found that a PD-1 dominant negative receptor (DNR) that, when cotransduced with a second-generation CAR, mediates enhanced T-cell persistence as well as T-cell resistance to tumor-mediated T-cell inhibition. The results disclosed herein demonstrate the benefit of optimizing signaling in an antigen-specific manner by simultaneously providing costimulation and check point blockade to counteract tumor-mediated T-cell inhibition. These results support using such an approach for improved tumor therapy, including but not limited to the treatment of MSLN-expressing solid tumors. 7.2. Methods and Procedures

The experimental procedures were approved by the Institutional Animal Care and Use Committee of Memorial Sloan Kettering Cancer Center (MSKCC). Each experiment was performed multiple times, using different donor T cells. To avoid confounding variables-such as differences due to transduction efficiencies, donor-related variability, and E:T ratios-data are presented using a representative experiment, with sample replicates of more than 3.

Cell lines. MSTO-211H human pleural mesothelioma cells (ATCC, Manassas, VA) were retrovirally transduced to express GFP and firefly luciferase fusion protein (MSTO GFP-ftLuc⁺). These cells were then transduced with the human MSLN variant 1 subcloned into an SFG retroviral vector to generate MSTO MSLN⁺ GFP-ffLuc⁺. Similarly, A549 cells and 3T3 murine fibroblasts were transduced with human MSLN variant 1 alone to generate A549 MSLN⁺ and 3T3 MSLN⁺ cell lines. 3T3 cells were also cotransduced with PD-L1 to generate 3T3 MSLN⁺ PDL1⁺ cells.

γ-Retroviral vector construction and viral production. To generate MSLN-specific CARs, a cDNA encoding for a fully human scFv m912 specific for MSLN (provided by D. Dimitrov, National Cancer Institute at Frederick) (Feng et al., Mol. Cancer Ther. 8(5):1113-1118 (2009)), linked to the human CD8 leader domain and the CD8/CD3ζ, CD28/CD3ζ, or CD8/4-1BB/CD3ζ domain was engineered, as previously described (Zhong et al., Mol. Ther. 18(2):413-420 (2010)). The control PSMA-specific CAR was generated similarly, using a previously characterized PSMA-targeting scFv (Gade et al., Cancer Res. 65(19):9080-9088 (2005)). For construction of the PD-1 DNR, commercial gene synthesis was used to encode the extracellular portion of the PD-1 receptor (amino acids 1-151) fused to the CD8 transmembrane and hinge domains. The CAR sequence was inserted into the SFG γ-retroviral vector (provided by 1. Riviere, MSKCC) and linked to a P2A sequence to induce coexpression of the LNGFR reporter (truncated low-affinity nerve growth factor receptor) or, in the case of the PD-1 DNR, the mCherry fluorescent protein reporter (Markley et al., Blood 115(17):3508-3519 (2010); Papapetrou et al., Proc. Natd. Acad. Sci. USA 10⁶(31):12759-12764 (2009)). The CAR and PD-1 DNR encoding plasmids were then transfected into 293T H29 packaging cell lines to produce the retrovirus, as previously described (Hollyman et al., J Immunother. 32(2):169-180 (2009)).

T-cell isolation, gene transfer, and CD4/CD8 isolation. Peripheral blood leukocytes were isolated from the blood of healthy volunteer donors under an institutional review board-approved protocol. Peripheral blood mononuclear cells (PBMCs) were isolated by low-density centrifugation on Lymphoprep (Stem Cell Technology, Vancouver, Canada) and activated with phytohemagglutinin (2 g/mL; Remel, Lenexa, KS). Two days after isolation, PBMCs were transduced with 293T RD114-produced retroviral particles encoding for CARs and PD-1 DNR and spinoculated for 1 h at 3000 rpm on plates coated with retronectin (15 μg/mL; r-Fibronectin, Takara, Tokyo, Japan). After 1 day, transduced PBMCs were maintained in IL-2 (20 UI/mL; Novartis, Basel, Switzerland). Transduction efficiencies were determined by flow cytometric analysis. Pure populations of CD4⁺ and CD8⁺ CAR⁺ T cells, or mCherry-positive PD-1 DNR-expressing and mCherry-positive EV-expressing CAR⁺ T cells, were obtained by flow cytometric-based sorting (BD Aria Sorter; BD Biosciences, San Jose, CA).

Flow cytometry. Human MSLN expression was detected using a phycoerythrin-or allophycocyanin-conjugated anti-human MSLN rat IgG2a (R&D Systems, Minneapolis, MN). Expression of costimulation or inhibitory proteins on tumor cells was analyzed using the following antibodies: 4-1BBL (PE, clone 5F4; BioLegend, San Diego, CA), MHC HLA-DR (PE, clone L203; R&D Systems), PD-L1 (APC, clone MIH1; eBioscience, San Diego, CA), PD-L2 (APC, clone MIH18; eBioscience), and galectin-9 (APC, clone 9M13; BioLegend). T-cell phenotype and transduction efficiency were determined with monoclonal antibodies for CD3, CD4, CD8, and CD69m LNGFR. Expression of T-cell inhibitory receptors was analyzed using PD1 (APC, eBioJIU5; eBioscience), TIM-3 (PE, clone 344823; R&D Systems), and Lag-3 (PE, clone C9B7W; BioLegend). Cell staining was analyzed using a BD LSRII flow cytometer (BD, Franklin Lakes, NJ) and FlowJo analysis software (FlowJo, Ashland, OR).

T-cell functional assays. The cytotoxicity of T cells transduced with a CAR or vector control was determined by standard⁵¹Cr-release assays, as previously described (McCoy et al., National Cancer Institute Monograph 37:59-67 (1973)). To perform the luciferase-activity assay, CAR⁺ T cells and MSTO-211H cells expressing MSLN and firefly luciferase were incubated for 18 h at different E:T ratios. Tumor-cell quantity was determined by BLI using IVIS 100/lumina II, after the addition of 100 μL of D-luciferin (15 mg/mL) per well, and was compared to the signal emitted by the tumor cells alone. CD10⁷a and intracellular staining were performed after incubation of effector cells and irradiated MSTO-21 1H MSLN tumor cells for 18 h in 24-well plates at a ratio of 5:1. For the CD10⁷a assay, 5 μL of CD10⁷a-PeCy7 antibody (BD Biosciences, San Jose, CA) and Golgi STOP (4 μL/6 mL; BD Biosciences) were added at the time of stimulation. For intracellular staining, Golgi Plug (1 L/1 mL; BD Biosciences) was added at the time of stimulation. After incubation, effector cells were stained for CD4, CD8, LNGFR, and CD3 marker, then fixed and permeabilized in accordance with the manufacturer's instructions (Cytofix/Cytoperm Kit; BD Biosciences). Staining for intracellular cytokines was performed using granzyme B-APC, perforin-PE, and IFN-γ-FITC antibodies (BD Biosciences).

Cytokine-release assays were performed by coculturing 3×10⁴ to 5×10³ T cells with target cells in a 1:1 to 5:1 ratio, in 200 μL of medium, in 96-well round-bottomed plates as triplicates. After 6 to 24 h of coculture, supernatants were collected. Cytokine levels were determined using a multiplex bead Human Cytokine Detection kit, in accordance with the manufacturer's instructions (Millipore, Darmstadt, Germany).

To analyze the proliferation capacity of T cells, 1×10⁶ CAR⁺ T cells were stimulated over irradiated MSTO-21 1H or 3T3 cells with or without MSLN expression (and, in the case of 3T3, with or without PD-L1). Proliferation assays were performed in the absence of exogenous IL-2. Cells were counted every 7 days and then overlaid on irradiated target cells for repeated stimulations. The CAR⁺ T cell number versus time was plotted for each T-cell group.

Orthotopic pleural mesothelioma animal model and ex vivo experiments. To develop the orthotopic mouse model of pleural mesothelioma, female NOD/SCIDγ mice (The Jackson Laboratory, Bar Harbor, Maine) aged 4 to 6 weeks were used. All procedures were performed under approved Institutional Animal Care and Use Committee protocols. Mice were anesthetized using inhaled isoflurane and oxygen, with bupivacaine administered for analgesia. Direct intrapleural injection of 1×10⁵ to 1×10⁶ tumor cells in 200 NL of serum-free medium via a right thoracic incision was performed to establish orthotopic MPM tumors, as previously described (Adusumilli et al., Science Translational Medicine 6(261):261ra151 (2014); Servais et al., Clin. Cancer Res. 18(9):2478-2489 (2012); Servais et al., in Current Protocols in Pharmacology, Enna, ed., Chapter 14 (Unit14 21), John Wiley & Sons (2011)). In total, 3×10⁴ to 1×10⁵ transduced T cells (in 200 μL of serum-free medium) were adoptively transferred into tumor-bearing mice, either into the thoracic cavity by direct intrapleural injection or systemically by tail vein injection. Tumor growth was monitored and quantified in vivo by BLI performed 20 minutes after a single intraperitoneal dose of D-luciferin (150 mg/kg; Perkin Elmer, Waltham, MA). BLI data were analyzed using Living Image software (version 2.60; Perkin Elmer); BLI signal was reported as total flux (photons per second), which represents the average of ventral and dorsal flux. To analyze the functional capacity of CAR T cells ex vivo, tumor tissues and mouse spleen were processed as follows: Tissues were weighed and harvested into ice-cold RPMI 1640. The tissues were manually morselized with a scalpel and then mechanically disaggregated through 40—to 100-μm filters. Next, samples were analyzed by FACS (fluorescence activated cell sorting) for phenotyping, or CAR⁺ CD4⁺ or CD8⁺ T cells were sorted using a FACS Aria sorter then rested for 24 h in RPMI with IL-2 (60 UI/mL), and ⁵¹Cr-release and cytokine-release assays were performed as described above.

Histologic analysis and immunostaining. Histopathologic evaluation of tumors was performed after hematoxylin and eosin (H&E) staining of paraffin-embedded, 4% paraformaldehyde-fixed tissue samples. Immunohistochemical analysis for human MSLN was performed with mouse anti-human MSLN immunoglobulin G, as previously described (Kachala et al., Clin. Cancer Res. 20(4):1020-1028 (2014); Rizk et al., Cancer Epidemiol. Biomarkers Prev. 21(3):482-486 (2012); Tozbikian et al., PLoS One 9(12):e114900 (2014)).

Quantitative Real-time PCR. The mRNA from CD4⁺ LNGFR+ or CD8⁺ LNGFR+ sorted T cells were extracted and reverse transcribed into cDNA using pMACS One-Step cDNA kit (MACS molecular, Miltenyi Biotech Inc, Auburn, USA). Quantitative Real Time PCR (RT-PCR) was performed with the Taqman® method using Applied Biosystems® 7500 systems (Foster, CA, USA), Tagman@Universal PCR Mastermix and Taqman® probes labeled with 6-carboxyfluorescein (FAM-MBG) and designed by Life Technologies (Carlsbad, CA): Tbet (Hs00203436_ml); Eomes (Hs00172872_ml); Granzyme B (Hs01554355_ml); IFN-γ (Hs00989291_ml); IL-2 (Hs00174114_ml); PD-1 (Hs01550088_ml). The comparative threshold cycle (CT) of the gene of interest was used and normalized to the P2m housekeeping gene using the following formula: ΔCt (sample)=Ct (gene of interest)-Ct (02m). Then, the 2^{-ΔΔCt} method was used to analyze the relative fold change expression compared to control condition and calculated as follow: 2^{-ΔΔCt}=2\hat{ }−(ΔCt(sample)−ΔCt(control)).

Statistical methods. Data were analyzed using Prism (version 6.0; GraphPad Software, La Jolla, CA) software and are presented as mean±SEM, as stated in the figure legends. Results were analyzed using the unpaired Student's t test (two-tailed), with the Bonferroni correction used for multiple comparisons, when applicable. Survival curves were analyzed using the log-rank test. Statistical significance was defined as P<0.05. All statistical analyses were performed with Prism software.

7.3. CARs with CD28 or 4-1BB Costimulation Exhibit Equivalent Effector Cytokine Secretion and Proliferation In Vitro Upon Initial Antigen Stimulation

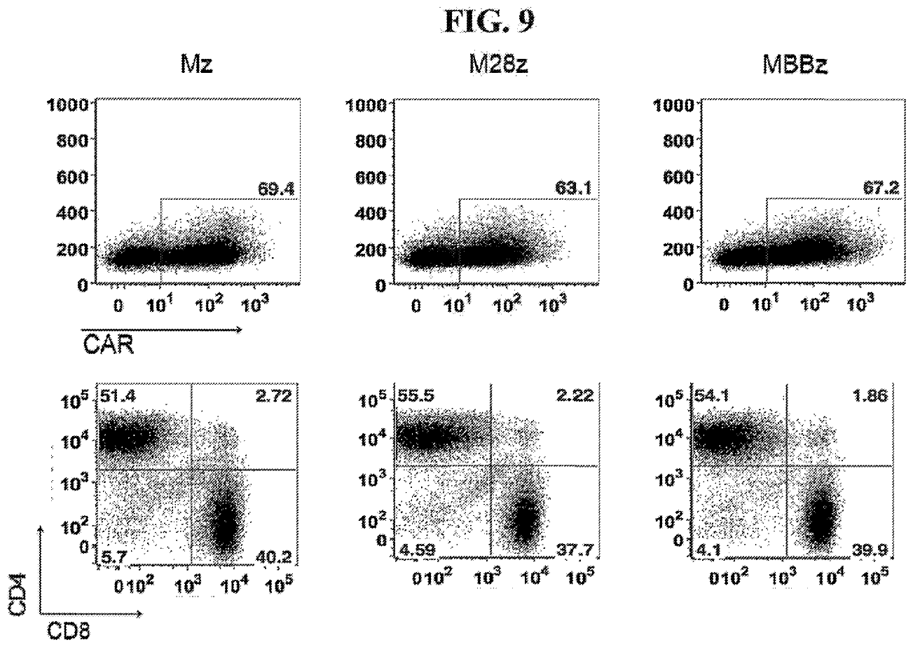

Three CARs were constructed that incorporated a human MSLN-specific scFv (Feng et al., Mol. Cancer Ther. 8(5):1113-1118 (2009)) and either CD3ζ, CD28/CD3c or 4-1BB/CD3ζ signaling domains (Mz, M28z, MBBz) (FIGS. 1A and 1B). The P28z CAR, which is specific for prostate-specific membrane antigen (PSMA), served as a negative effector to control for alloreactivity and xenoreactivity. Both CD4⁺ and CD8⁺ human peripheral blood T lymphocytes were effectively transduced using the SFG-retroviral vector (50%-70% transduction) (FIG. 9). MSLN-transduced MSTO-21 1H cells (MSLN⁺) and PSMA-transduced EL-4 mouse lymphoma cells (MSLN-) served as MSLN-positive and—negative targets in the in vitro experiments. Mz-, M28z-, and MBBz-transduced T cells demonstrated similar MSLN-specific lysis in vitro (FIG. 1C). P28z CAR T cells did not lyse MSTO MSLN⁺ cells, and MSLN-targeted CARs did not lyse EL4 PSMA+ cells, demonstrating that lysis is antigen specific. Validating the functionality of costimulatory signaling (Brentjens et al., Clin. Cancer Res. 13(18 Pt 1):5426-5435 (2007)), M28z and MBBz CAR T cells secreted 2—to 15-fold higher levels of Th1 cytokines (FIG. 1D) and achieved 14-fold greater T-cell accumulation upon repeated exposure to MSLN⁺ cells when compared to Mz in the absence of exogenous IL-2 (FIG. 1E). Having established antigen specificity and validated the functionality of costimulatory signaling domains, evaluation of the therapeutic potential of MSLN-targeted CAR T cells in mice bearing established pleural tumors was performed.

These results demonstrate that CARs with CD28 or 4-1BB costimulation exhibit equivalent effector cytokine secretion and proliferation in vitro upon initial antigen stimulation.

7.4. M28z is More Prone to Allowing Tumor Relapse than MBBz

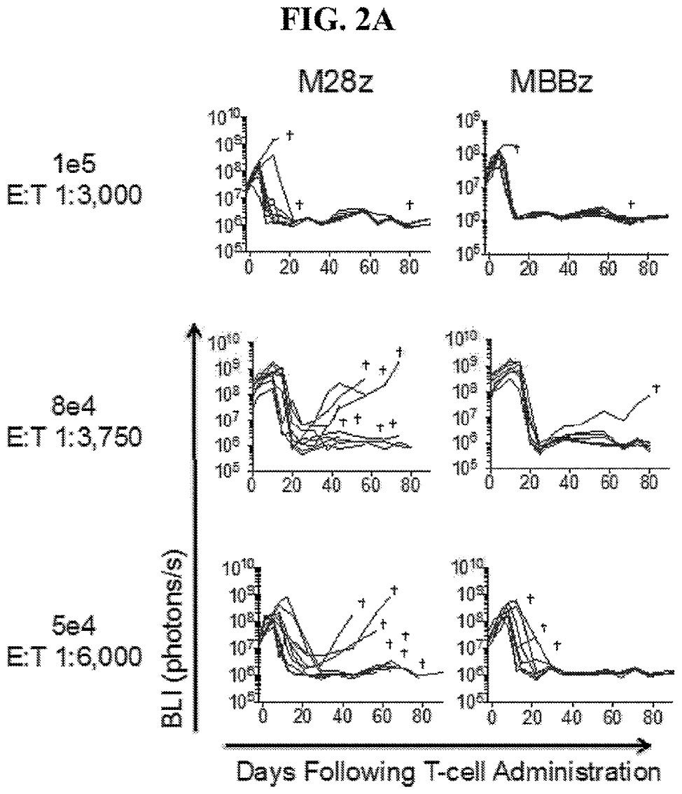

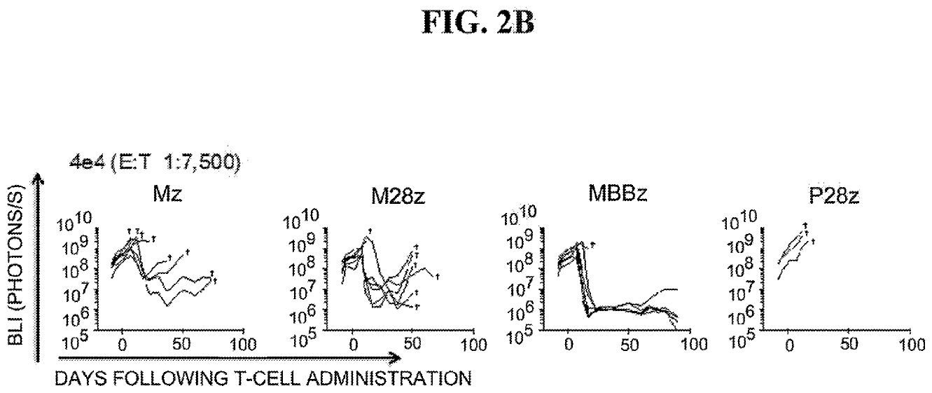

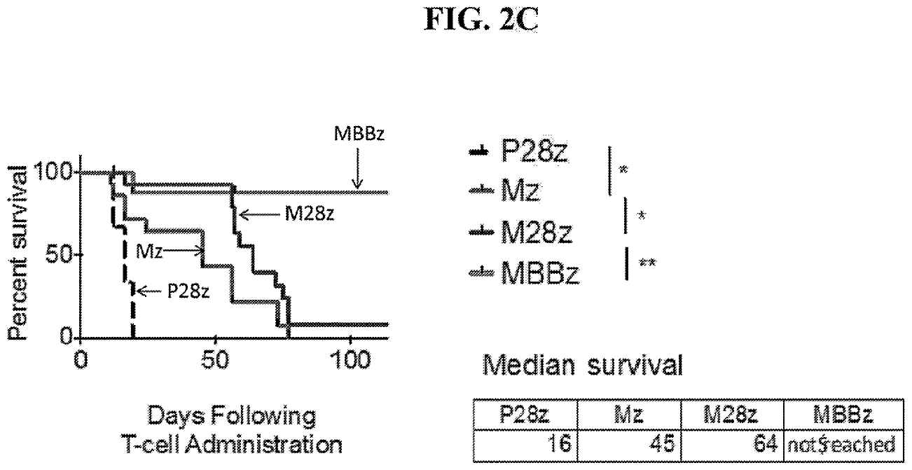

In an orthotopic model of malignant pleural mesothelioma (MPM) previously established (Servais et al., Clin. Cancer Res. 18(9):2478-2489 (2012); Servais et al., in Current Protocols in Pharmacology, Enna, ed., Chapter 14 (Unit14 21), John Wiley & Sons (2011); Servais et al., PLoS One 6(10):e26722 (2011); Adusumilli et al., J. Gene Med. 8(5):603-615 (2006)), serial bioluminescence imaging (BLI) with firefly-luciferase (ffLuc)-transduced MSTO-211H cells was used to confirm the establishment of tumor, to equalize tumor burden across intervention groups before the initiation of T-cell therapy, and to measure the response to therapy. Both M28z and MBBz CAR T cells intrapleurally administered at a single dose of 1×10⁵ (effector to target (E:T) ratio of 1:3000, estimated from tumor burden quantification) (Servais et al., PLoS One 6(10):e26722 (2011)) are able to eradicate established pleural tumors in the majority of mice (FIG. 2A, top).

Since the goal in this study was to investigate the effect of tumor-induced immuno inhibition on T-cell exhaustion, CAR T cells were administered to mice bearing established pleural tumors at successively lower doses. At these lower doses, it was expected that T cells would be especially susceptible to exhaustion as they must retain function upon repeated antigen encounters within an inhibitory environment in order to eliminate tumor. It is at these lower doses tumor relapse was begun to be observed, especially within the M28z cohort (FIG. 2A, middle and bottom). At the lowest dose tested of 4×10⁴ (E:T, 1:7,500), mice treated with intrapleural Mz (first generation CAR, no costimulatory signaling included) CAR T cells showed an unsustainable response in terms of tumor burden (FIG. 2B), and median survival was 29 days longer than that in the P28z-treated controls (median survival, 45 vs. 16 days, P28z represents a xenoreactivity and alloreactivity control targeting the PSMA antigen) (FIG. 2B). Mice treated with M28z CAR T cells had a more uniform reduction in tumor burden and survived longer (median survival, 64 days) than mice treated with first-generation CAR T cells; however, all mice treated with M28z CAR T cells eventually died of progressing tumor. It was confirmed that tumor outgrowth was not caused by tumor antigen escape (recurring tumors in all tested mice were found to be MSLN⁺ by flow cytometric and histologic analysis). In contrast, intrapleurally administered MBBz CAR T cells induced tumor eradication within 20 days of treatment, and the vast majority of mice (7 of 8) remained tumor free for >100 days (median survival was not reached by day 100).

These results demonstrate that M28z is more prone to allowing tumor relapse than MBB.

### 7.5. MBBz Surpasses M28z CAR T Cells at Low T-cell Doses

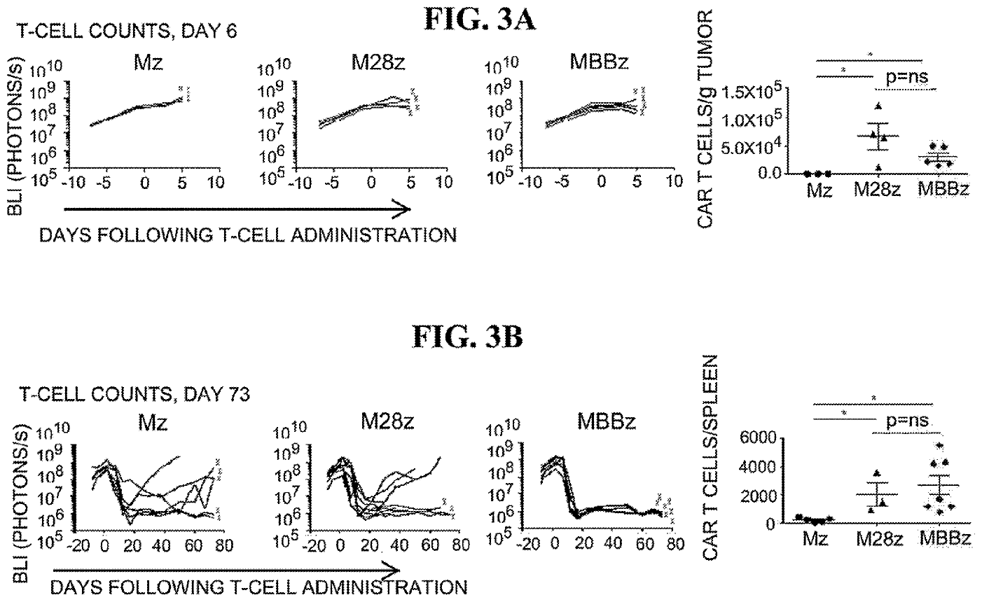

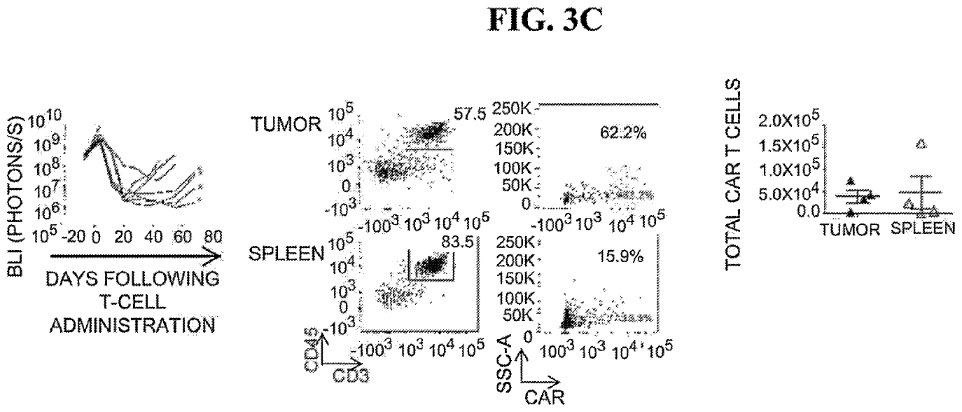

Improvements in CAR T-cell efficacy afforded by costimulatory signaling are typically attributed to improvements in CAR T-cell proliferation and/or persistence (Sadelain et al., Cancer Discovery 3(4):388-398 (2013)). As expected, M28z and MBBz CAR T cells achieved enhanced intratumoral T-cell accumulation, compared with Mz CAR T cells (9-fold greater for M28z, 12-fold greater for MBBz) (FIG. 3A). Surprisingly, despite the differences in efficacy between M28z and MBBz CAR T cells, similar numbers of tumor-infiltrating T cells were observed between the two groups (FIG. 3A). Furthermore, M28z and MBBz CAR T cells were equally persistent at long-term time points (FIG. 3B). Tumor tissue and spleen from M28z-treated mice that initially had a treatment response but then died of progressing tumor contained circulating T cells as well as tumor-infiltrating T cells, including CAR positive cells (FIG. 3C). This finding demonstrates that the mere persistence of T cells that can effectively traffic to the tumor is not sufficient to eliminate tumor and that the T-cell functional status within the tumor microenvironment may be the more critical determinant of clinical outcome. These results suggested that even costimulated T cells may become exhausted within a tumor, especially at low T-cell doses that correspond to low effector:target ratios. Furthermore, MBBz CAR T cells, which were as persistent as M28z CAR T cells, may be better able to resist exhaustion and retain T-cell effector function in order to eliminate a large tumor burden.

These results demonstrate that MBBz surpasses M28z CAR T cells at low T-cell doses.

### 7.6. Mesothelin CAR T Cells Become Exhausted Following In Vivo Antigen Exposure

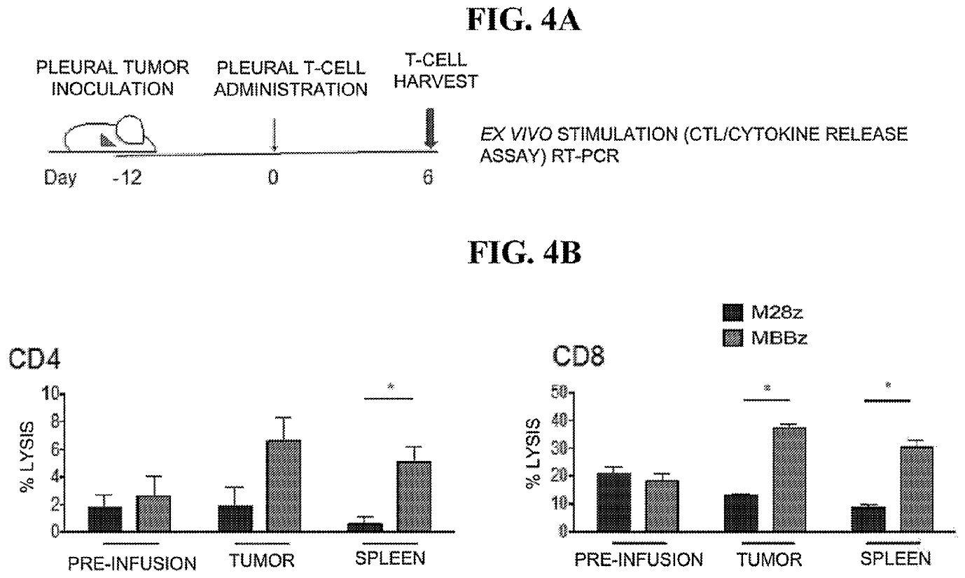

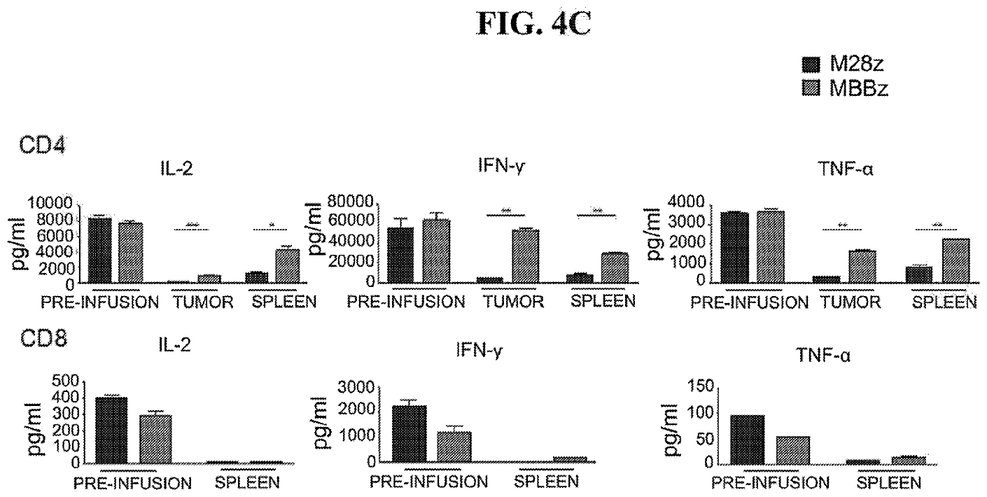

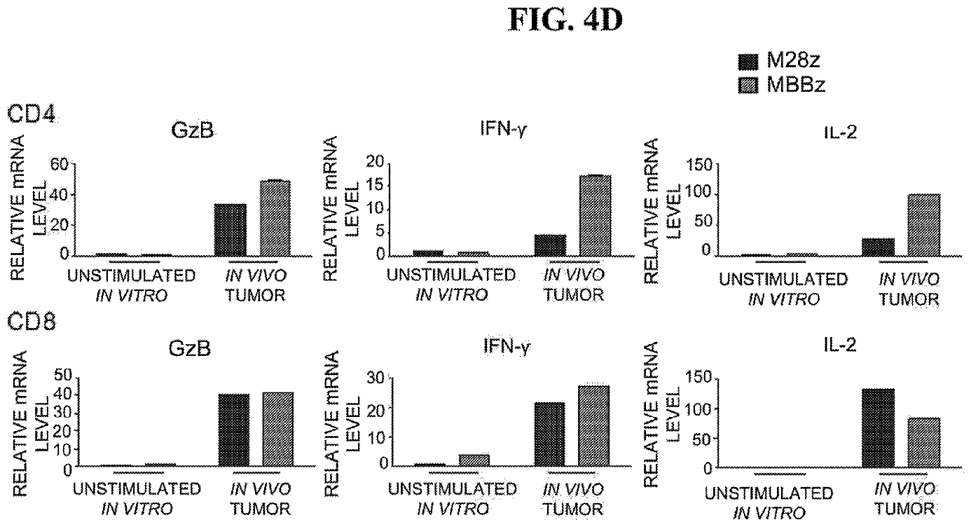

To assess whether there is ongoing immuno inhibition of CAR T cells and to compare the relative abilities of M28z and MBBz CAR T cells to overcome tumor-mediated immuno inhibition, 1×10⁶ CAR T cells were injected into the pleural cavities of MSTO MSLN⁺ tumor-bearing mice, allowed sufficient time for repeated antigen encounter and T-cell activation (confirmed by forward- and side-scatter and upregulation of the activation marker CD69), and then performed ex vivo stimulation of harvested CD4 or CD8 CAR tumor-infiltrating or splenic T cells with MSLN⁺ targets (schematic shown in FIG. 4A). Uninjected in vitro resting T cells (“preinfusion cells”) were used to establish the baseline level of function (before antigen exposure). Compared with resting M28z CD8⁺ CAR T cells, T cells exposed to MSLN antigen in vivo had lower levels of cytolytic function (FIG. 4A) (preinfusion cell lysis, 20.5%; tumor-infiltrating T-cell lysis, 13.1%; splenic T-cell lysis, 8.7%). In contrast, MBBz CAR T cells retained cytolytic function (preinfusion cell lysis, 18.3%; tumor-infiltrating T-cell lysis, 37.2%; splenic T-cell lysis, 22.2%). Sorted CD4⁺ CAR T cells demonstrated a similar pattern of results.

Cytokine levels were also measured upon ex vivo stimulation of tumor-infiltrating and splenic CAR T cells, and a decrease in Th1 cytokine secretion was observed for CD4⁺ M28z CAR T cells exposed in vivo to MSLN⁺ antigen. CD4⁺ MBBz CAR T cells also demonstrated a decrease in Th1 cytokine secretion, although these cells were better able to retain cytokine secretion when compared with M28z CAR T cells (FIG. 4B). CD8⁺ T cell supernatants contained significantly lower levels of cytokines, compared with CD4⁺ T cell supernatants (a finding previously observed Adusumilli et al., Science Translational Medicine 6(261):261ra151 (2014)). CD8⁺ T cells also had a decreased ability to secrete cytokines upon in vivo antigen exposure; CD8⁺ MBBz CAR T cells preferentially retained their ability to secrete IFN-γ. The mRNA levels of T cells harvested from tumor and spleen on day 3 after administration were assessed, and it was found that the in vivo expression levels of GzB, IL-2, and IFN-γ were mostly greater for CD4⁺ and CD8⁺ MBBz CAR T cells than for M28z CAR T cells, with the exception of IL-2 expression in the CD8⁺ subset (FIG. 4C).

These results demonstrate that mesothelin CAR T cells become exhausted following in vivo antigen exposure.

### 7.7. MBBz CAR T Cells Show Delayed Exhaustion In Vivo

The below describes experimental results showing that MBBz CAR T cells show delayed exhaustion in vivo.

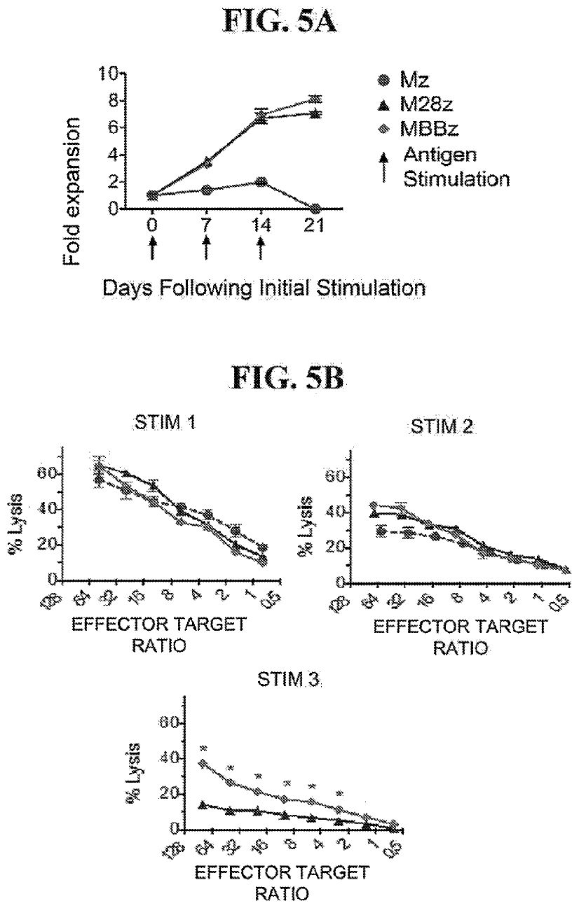

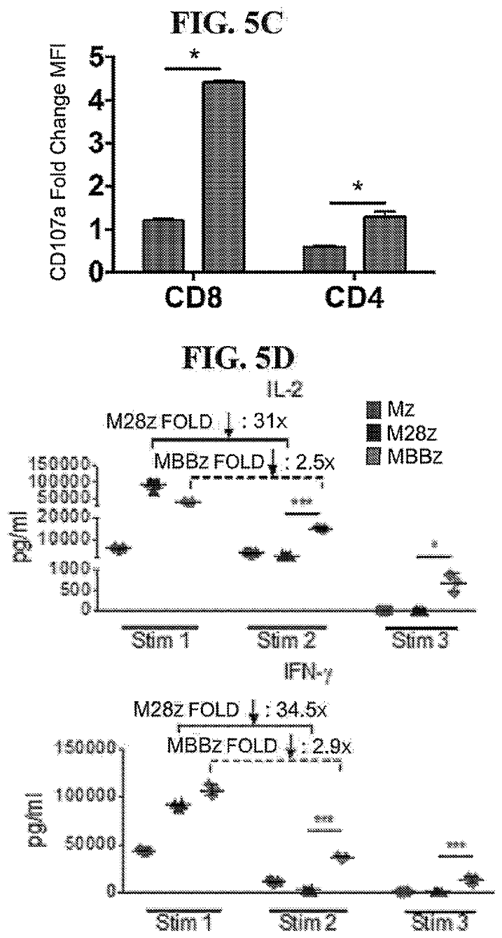

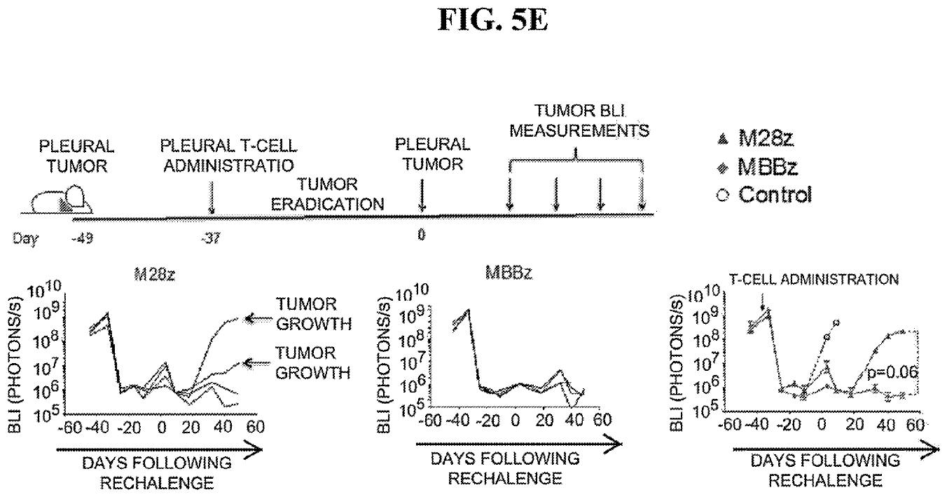

Having demonstrated inhibition of both the cytolytic function and effector cytokine secretion in costimulated CAR T cells exposed to antigen in vivo (see above), it was reasoned that repeated antigen stimulation may, similar to models of chronic infection, play a role in T-cell inhibition and that differing abilities to retain function upon repeated antigen encounter might explain enhanced efficacy of MBBz CAR T cells. Therefore, Mz, M28z, and MBBz CAR T cells were tested for their ability to withstand repeated antigen encounter in an in vitro model system, wherein cells were assessed for proliferation, cytolytic function, and cytokine secretion upon MSLN⁺ antigen stimulation every 7 days. M28z and MBBz CAR T cells had similar abilities to expand upon serial MSLN⁺ stimulation, expanding to levels 14-fold greater than those of Mz CAR T cells; they lost the ability to expand following the third stimulation (FIG. 5A). Both MBBz and M28z CAR T cells lost cytolytic function upon repeated antigen stimulation, although MBBz CAR T cells were better able to retain lytic function. Whereas lysis was equal among the three T-cell groups at the first stimulation, by the third stimulation, M28z lytic function was inhibited to a more pronounced level, such that MBBz CAR T cells had enhanced tumor lysis at multiple E:T ratios (FIG. 5B, right). Lytic function (as assessed by a degranulation assay measuring CD10⁷a expression) at the third stimulation correlated with the results of chromium-release assays (FIG. 5C).

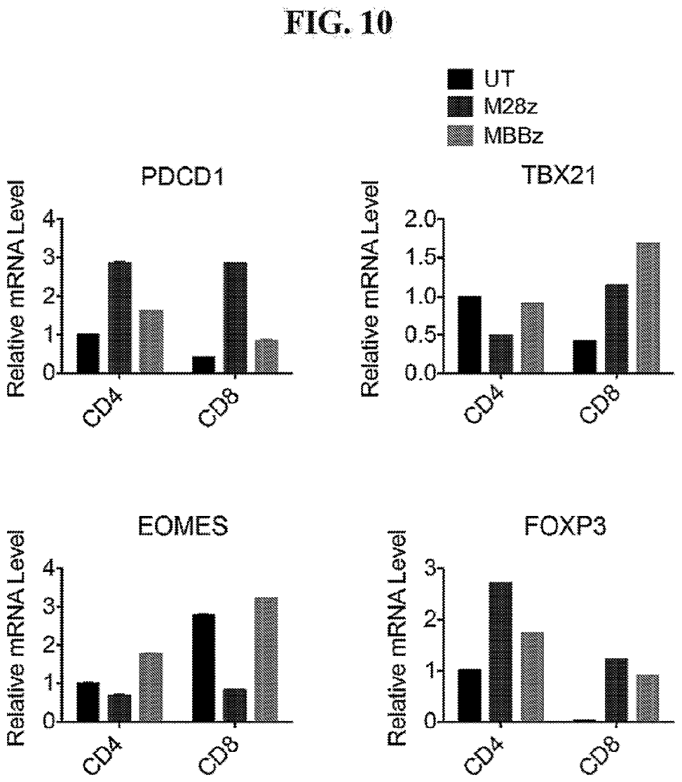

Next, Th1 cytokine secretion was measured. Similar levels between M28z and MBBz CAR T cells were noted at the first stimulation, as well as a successive decrease with each stimulation. As with cytotoxicity, MBBz CAR T cells preferentially retained cytokine secretion; cytokine concentrations decreased >30-fold for M28z and only around 2-fold for MBBz CAR T cells, when levels at the first and second stimulations were compared (FIG. 5D). The differences in cytokine production were confirmed by measuring intracellular levels of cytokines at the second stimulation. Reverse-transcriptase PCR analysis of CAR T cells at the time of antigen stimulation revealed that MBBz CAR T cells expressed markers that correlate with lower levels of exhaustion and inhibition, compared with M28z CAR T cells; MBBz CAR T cells expressed higher levels of Tbet and Eomesodermin and lower levels of PD1 and FoxP3 (FIG. 10). The in vivo function of persisting CAR T cells that had already been exposed to tumor antigen was tested. Although quantitative persistence is equal between M28z and MBBz CAR T cells, it was thought that MBBz CAR T cells would demonstrate enhanced function upon tumor rechallenge. Mice with established MSLN⁺ pleural tumors were administered intrapleural M28z or MBBz CAR T cells (at a dose of 1×10⁵, E:T ratio 1:3000) to eradicate pleural tumor (FIG. 5E). Twenty days after the initial T-cell injection, tumor rechallenge was performed by injecting MSLN⁺ tumor cells (1×10⁶) into the pleural cavity of survivors; tumor burden was monitored using BLI. Persisting MBBz CAR T cells were better able to control tumor burden (4 of 4 MBBz-treated mice had a BLI signal at baseline levels vs. 2 of 4 M28z-treated mice) (FIG. 5E).

These results demonstrate that MBBz CAR T cells show delayed exhaustion in vivo.

### 7.8. Tumor Cell PD-L1 Inhibits Mesothelin CAR T-Cell Effector Functions

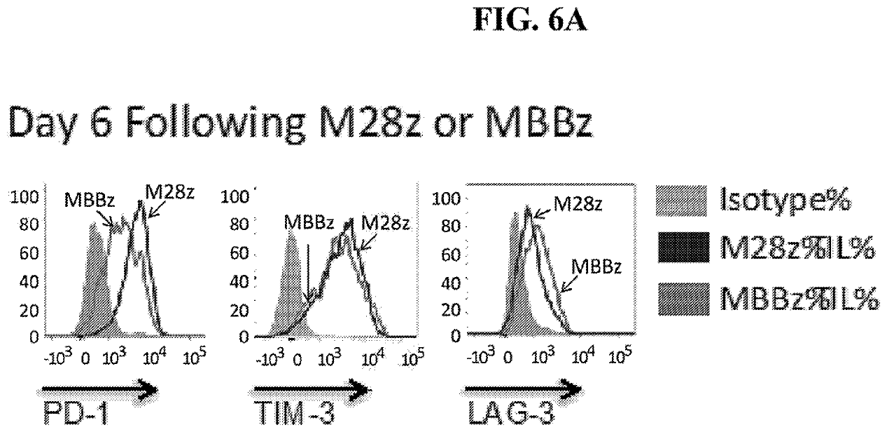

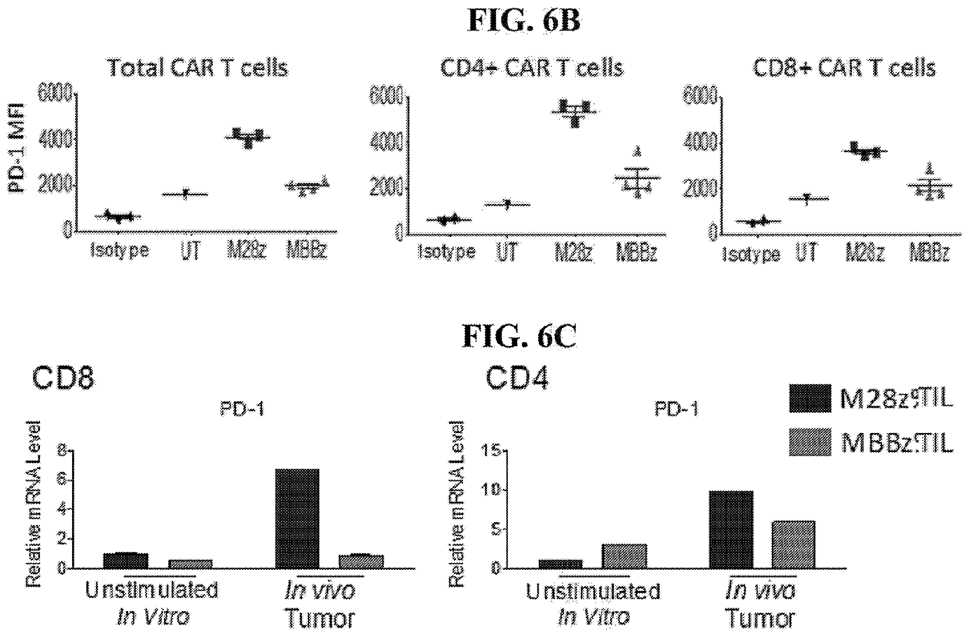

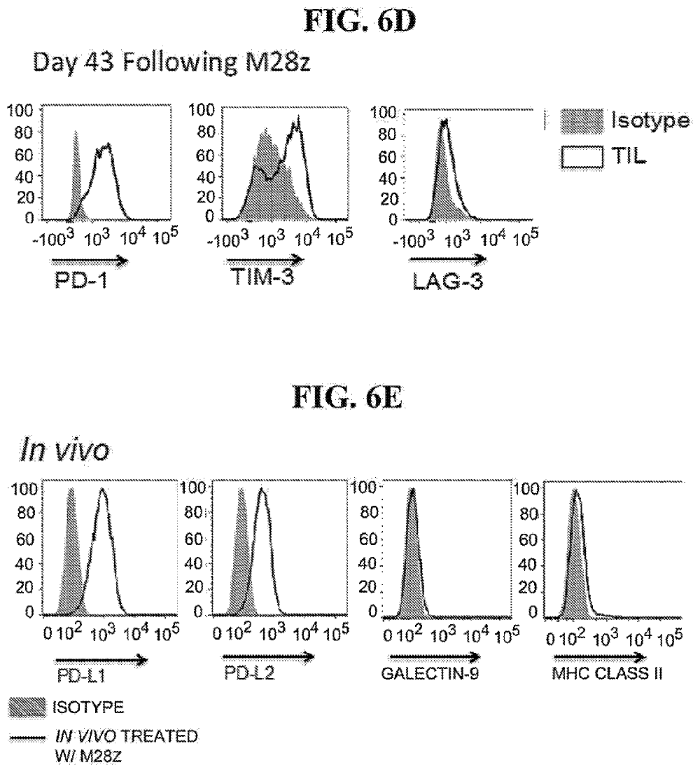

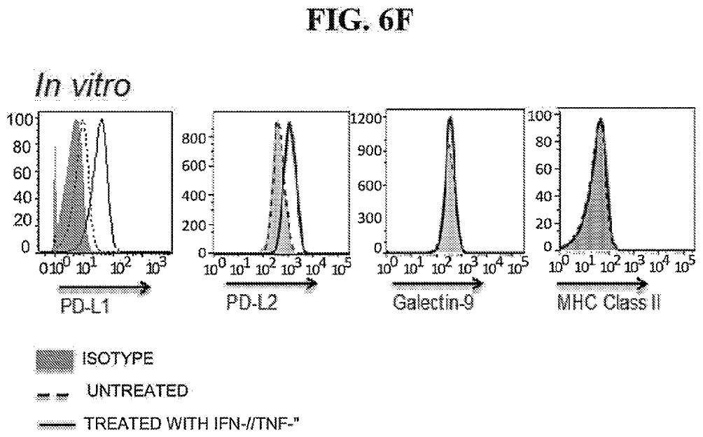

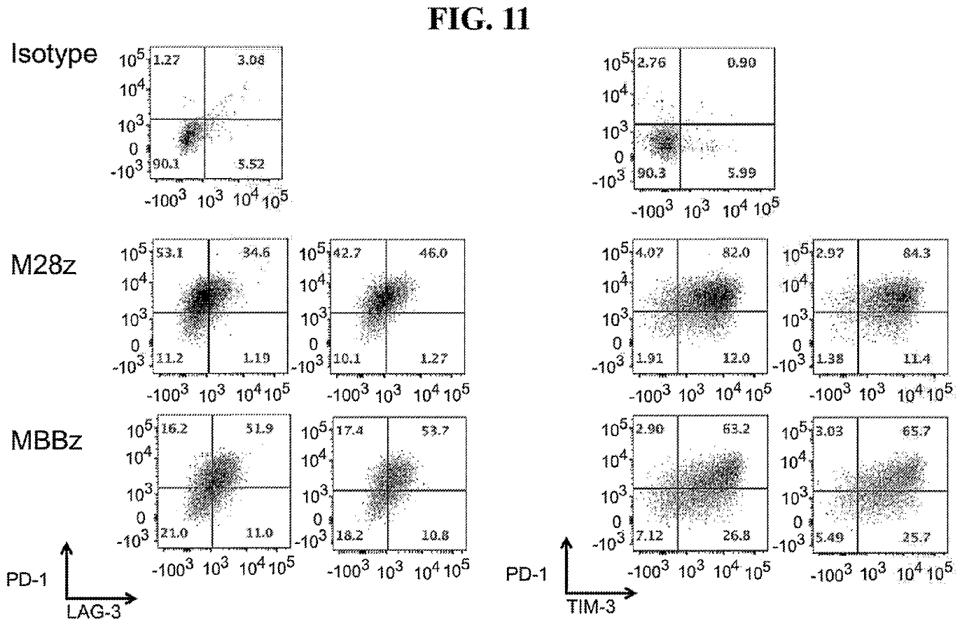

Having established that CAR T cells are inhibited by the in vivo tumor environment and that MBBz CAR T cells are better able to overcome this inhibition, at least in part because of their ability to retain function upon repeated antigen encounter (see above), it was next sought to assess the role that inhibitory receptor and ligand pathways play in the model. Tumor-infiltrating T cells, in M28z-treated mice with tumor progression, were stained for the expression of well-known pathways of inhibition. High levels of expression of PD-1, Tim-3, and LAG-3 were found (FIG. 6A). Tumor-infiltrating MBBz CAR T cells harvested 6 days after administration demonstrated upregulation of inhibitory receptors as well, although they expressed significantly lower levels of PD-1 receptor at both the protein and the mRNA level (FIG. 6B-D). CD4⁺ T cells expressed higher levels of PD-1, compared with CD8⁺ T cells. It was also observed that a significant fraction of both M28z and MBBz CAR T cells coexpressed PD-1 and LAG-3 or PD-1 and Tim-3, suggesting that multiple inhibitory pathways could be functioning simultaneously (FIG. 11). Next, tumor-expressed ligands were assessed: PD-L1 and PD-L2 (ligands for PD-1), galectin-9 (ligand for Tim-3), and MHC class II (ligand for LAG-3). Only PD-1 ligands were expressed on pleural tumor cells harvested after intrapleural administration of M28z CAR T cells (FIG. 6E). As reported elsewhere (McGray et al., Mol. Ther. 22(1):206-218 (2014); Spranger et al., Science Translational Medicine 5(200):200ra1 16 (2013)), coculture of tumor cells with IFN-γ and TNF-α (at concentrations similar to those secreted by T cells in FIGS. 1 and 5) resulted in a similar level of upregulation of PD-L1 and PD-L2 expression on tumor cells (FIG. 6F), reflecting an adaptation of tumor cells to resist immune attack (“adaptive immunoresistance”). The unique presence of expression of both PD-1 receptor and ligand in vivo suggests that this pathway may play a significant inhibitory role.

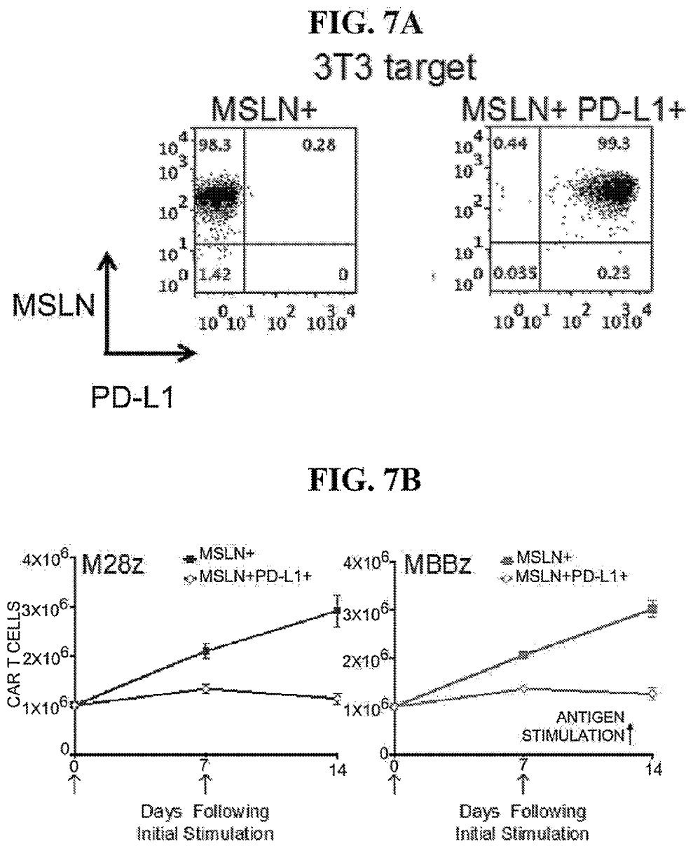

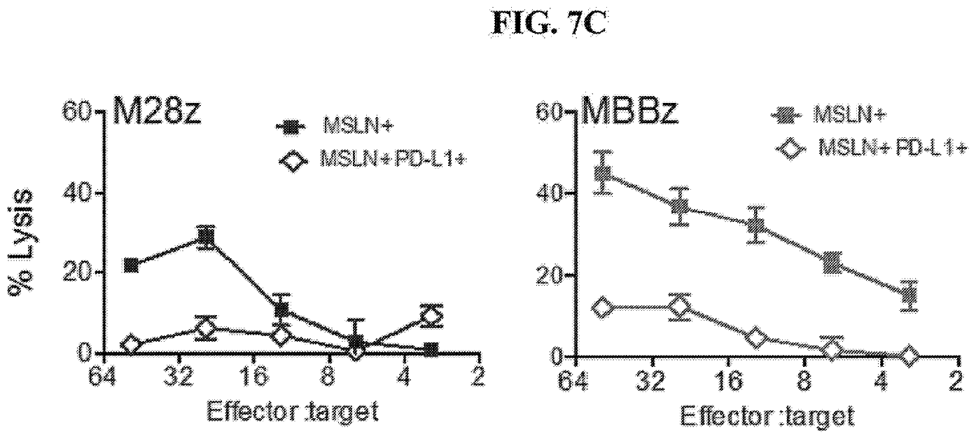

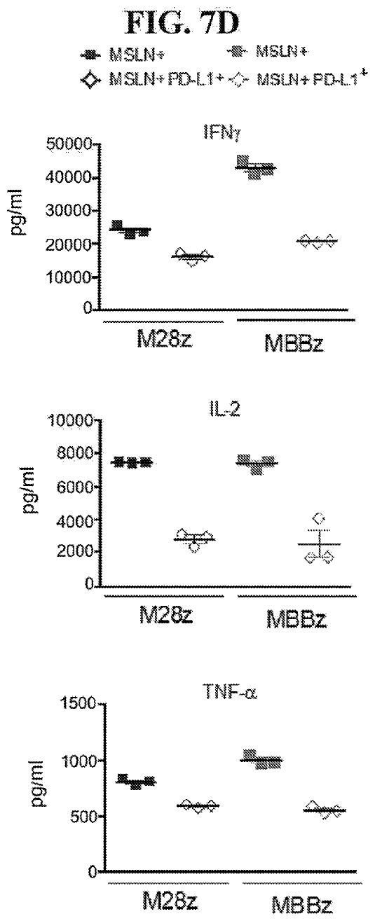

As some studies have suggested that costimulation may be sufficient to overcome inhibition by PD-1 (Carter et al., Eur. J. Immunol. 32(3):634-643 (2002); Freeman et al., J. Exp. Med. 192(7):1027-1034 (2000); Koehler et al., Cancer Res. 67(5):2265-2273 (2007)), it was next assessed whether overexpressed PD-L1 can inhibit CAR T-cell function in an in vitro model of PD-L1-mediated immuno inhibition (using 3T3 mouse fibroblasts transduced with either MSLN alone (MSLN⁺) or both MSLN and PD-L1 (MSLN⁺ PD-L1⁺)) (FIG. 7A). In both M28z and MBBz CAR T cells, PD-L1 overexpression resulted in decreased accumulation upon successive stimulation (FIG. 7B) and Th1 effector cytokine secretion (FIG. 7D). Although tumor-cell lysis was not inhibited upon initial stimulation, chromium release assay performed with 3T3s as targets following two stimulations against MSTO MSLN⁺ tumor cells demonstrates decreased lytic function in both M28z and MBBz CAR T cells, a higher extent of decrease in M28z CAR T cells (FIG. 7C). This result may be due to the differential upregulation of PD-1 on M28z and MBBz CAR T cells following exposure to MSTO MSLN⁺ tumor cells.

These results demonstrate that tumor cell PD-L1 inhibits mesothelin CAR T-cell effector functions.

### 7.9. Cell Intrinsic PD-1 Resistance Rescues M28z CAR T-Cell Function In Vivo

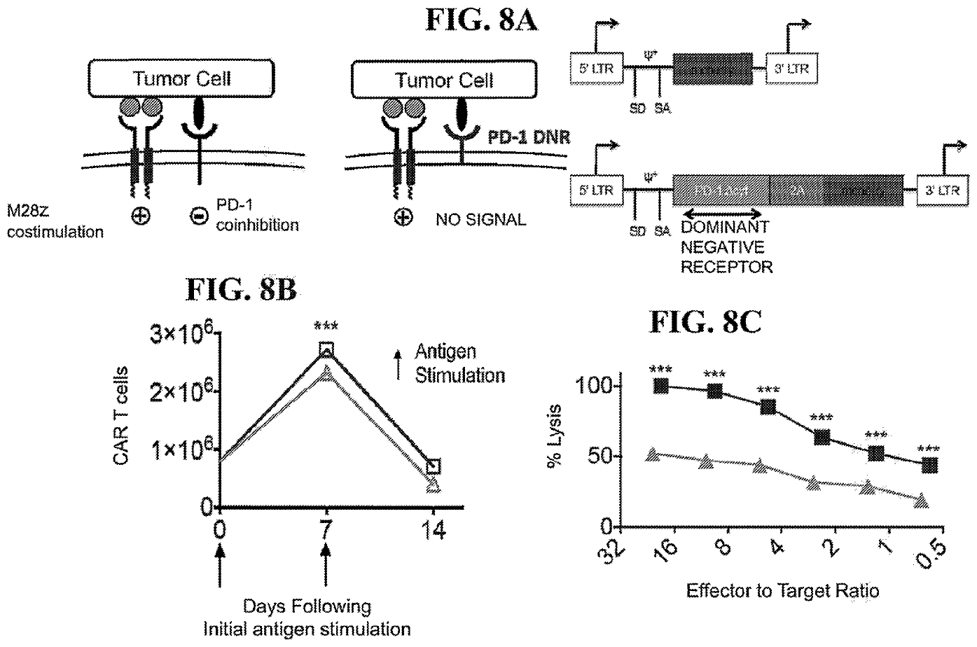

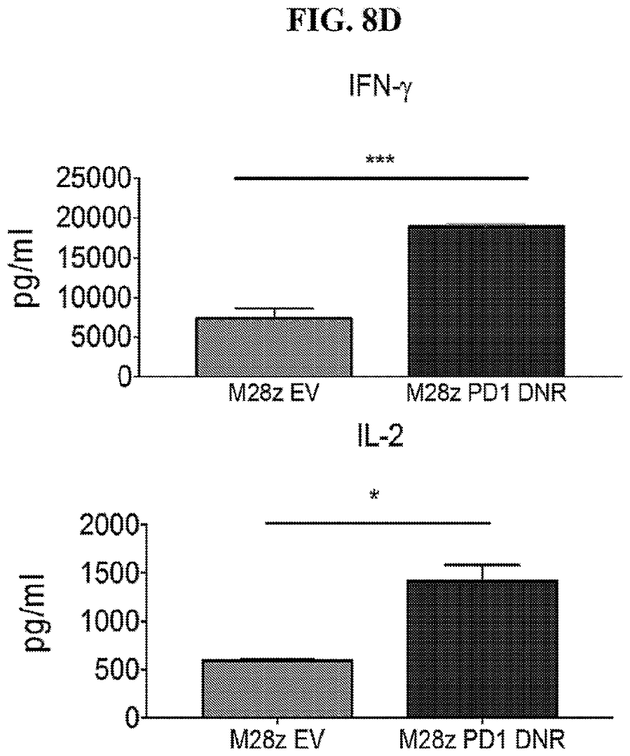

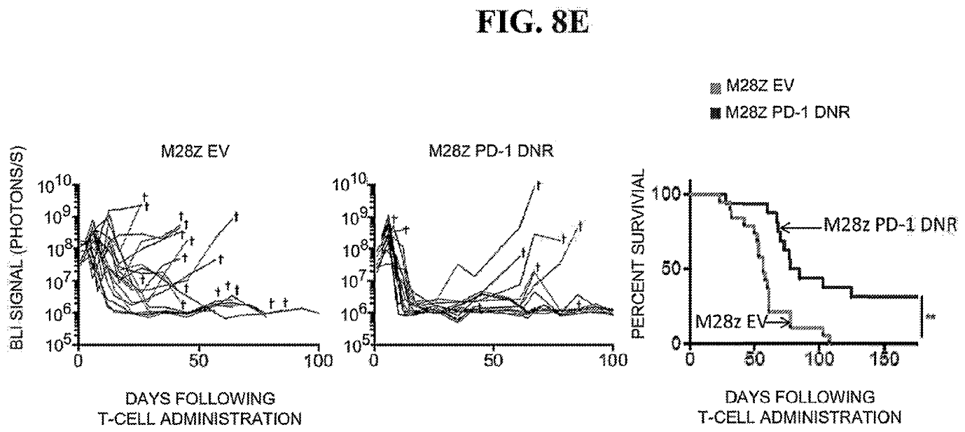

The above results indicate that the PD-1 pathway is a functioning mechanism of tumor-mediated immuno inhibition and that PD-1 upregulation following repeated antigen stimulation decreases CAR T-cell efficacy. Therefore, checkpoint blockade was combined with CD28 costimulatory signaling. Since the goal was to provide CAR T-cell-specific checkpoint blockade that was not reliant on repeated dosing of systemically administered antibodies, the studies were focused on genetically engineered methods of overcoming immuno inhibition. A PD-1 dominant negative receptor (DNR) was constructed that contained the extracellular ligand binding domain of the receptor fused to a CD8 transmembrane domain. Since the PD-1 DNR lacks any signaling domain, it was thought that sufficiently overexpressed receptor would enhance T-cell efficacy by saturating PD-1 ligands and thereby blocking signaling through the endogenous PD-1 receptor. M28z CAR T cells were cotransduced with either the PD-1 DNR linked by a P2A element to an mCherry reporter (PD-1 DNR) or an empty vector containing only the reporter (EV) (FIG. 8A). M28z CAR T cells cotransduced with the PD-1 DNR had slight but statistically significant advantages in proliferative ability (FIG. 8B), enhanced cytotoxicity (FIG. 8C) at multiple E:T ratios, as well as augmented levels of IL-2 and IFN-γ secretion (FIG. 8D).

Next, it was assessed whether intrapleural administration of M28z CAR T cells cotransduced with a genetically engineered PD-1 resistance would provide an in vivo advantage. Mice with established pleural MSLN⁺-expressing tumors were administered a single intrapleural dose of 5×10⁴ CAR⁺ M28z EV or M28z PD-1 DNR T cells, and treatment response was monitored by tumor burden measurements (using serial BLI) and median survival. Mice treated with M28z PD-1 DNR T cells had significantly enhanced tumor burden control and prolonged median survival (FIG. 8E); however, only some mice (7/16, 44%) had long-term tumor-free survival, suggesting that there are redundant mechanisms of immuno inhibition that must be overcome. A cohort of the mice (M28z PD-1 DNR) in this experiment survived beyond 450 days in spite of repeated tumor rechallenge, demonstrating the “functional persistence” of CAR T cells transduced with PD-1 DNR. These results demonstrate that, with an injection of 50,000 CAR T cells, not only was a large tumor burden eradicated but tumor relapse was prevented in spite of multiple tumor rechallenge over more than 15 months.

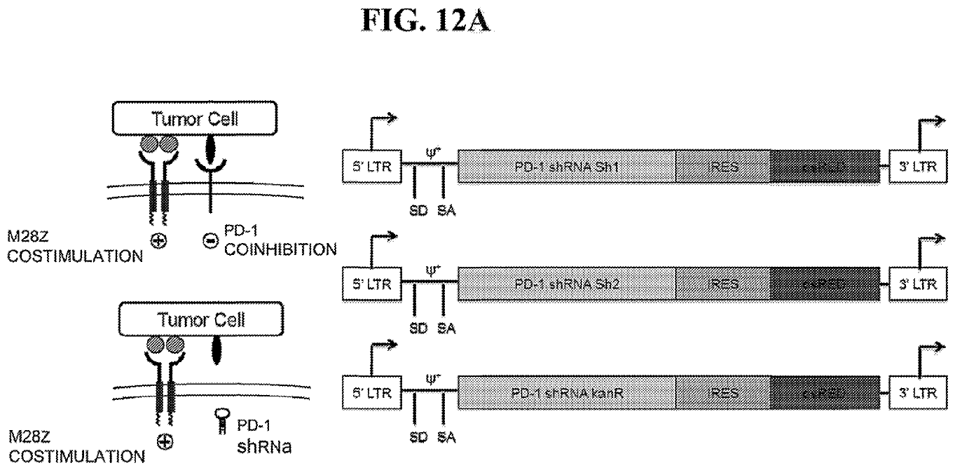

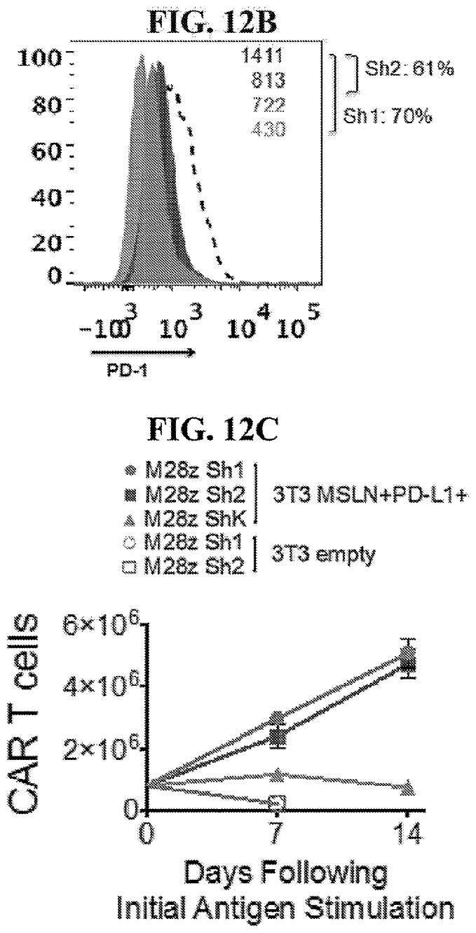

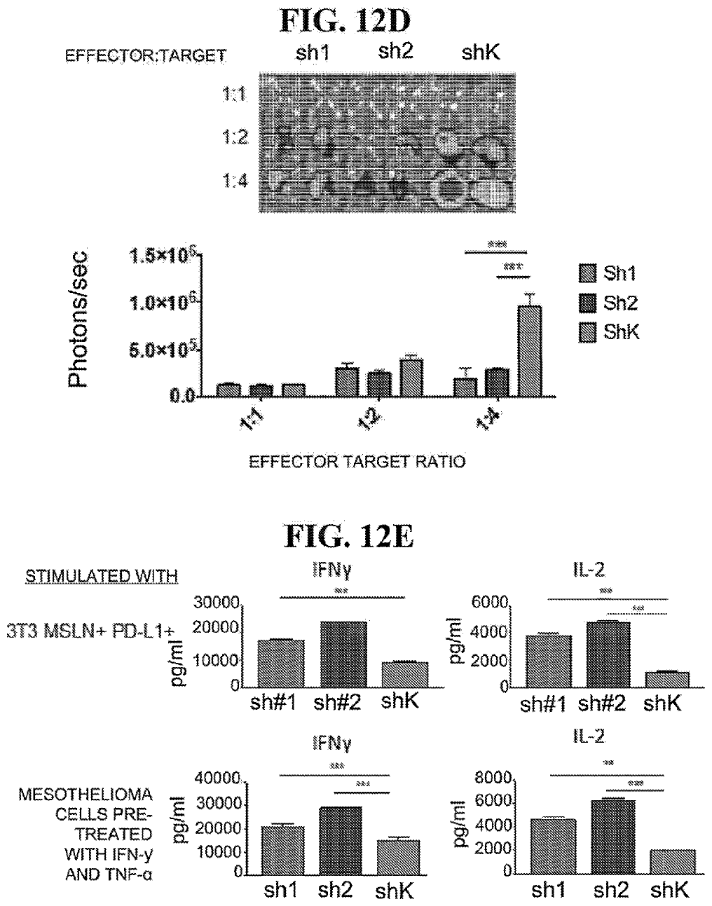

To investigate an alternative genetic strategy for overcoming PD-1-mediated immuno inhibition, M28z CAR T cells were cotransduced with vectors expressing PD-1-targeting shRNAs (FIG. 12A), which generated >60% PD-1 receptor knockdown at the protein level (FIG. 12B). In M28z CAR T cells, cotransduction with PD-1 shRNAs enhanced proliferative function upon MSLN⁺ antigen stimulation (FIG. 12C), augmented cytotoxicity (FIG. 12D), and enhanced cytokine secretion upon stimulation with either mesothelioma cells or MSLN⁺ PDL1+3T3 mouse fibroblasts (FIG. 12E), compared with cotransduction with an shRNA targeting a non-mammalian gene (M28z KanR). M28z PD-1 shRNA-transduced T cells did not achieve greater in vivo tumor rejection efficacy than M28z KanR T cells, but it is noteworthy that the level of knockdown was significantly lower in vivo than in vitro. Thus, the PD1 DNR proved to be the more effective strategy in vivo than the RNA interference approach.

These results demonstrate that cell intrinsic PD-1 resistance rescues M28z CAR T-cell function in vivo.

7.10. MBBz CAR T Cells Prolong Tumor-free Survival in a Mouse Model of Metastatic Lung Cancer in which PD-1 Receptor and Complementary Ligands are Expressed

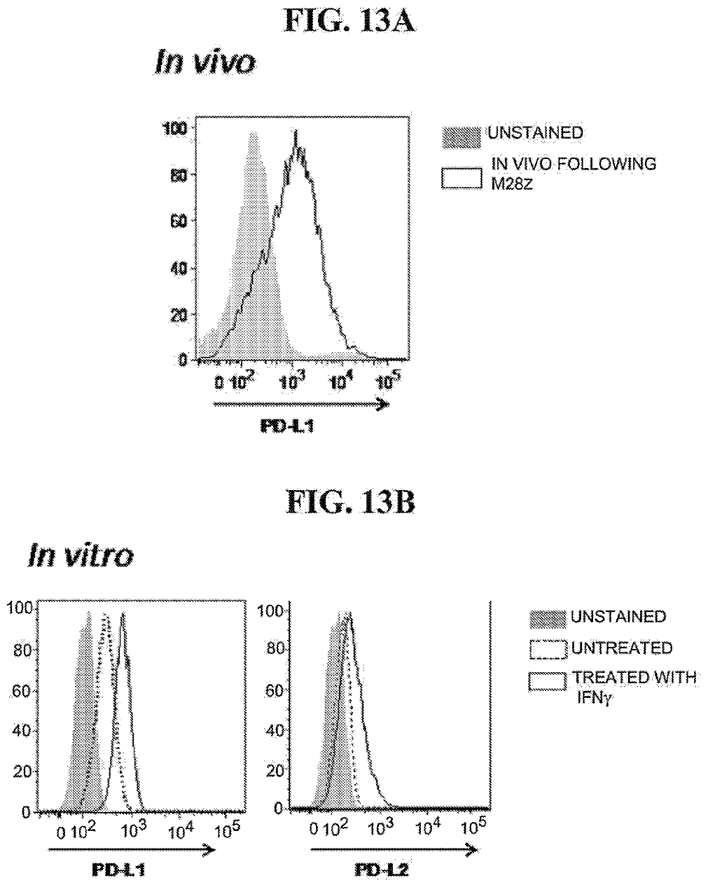

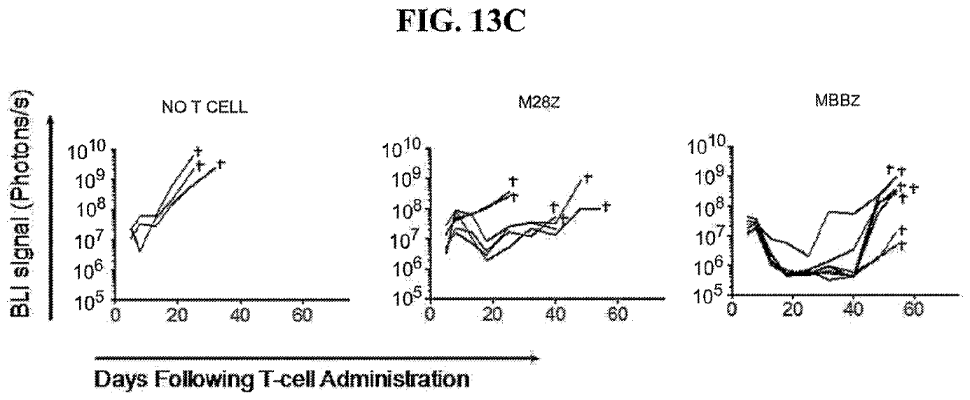

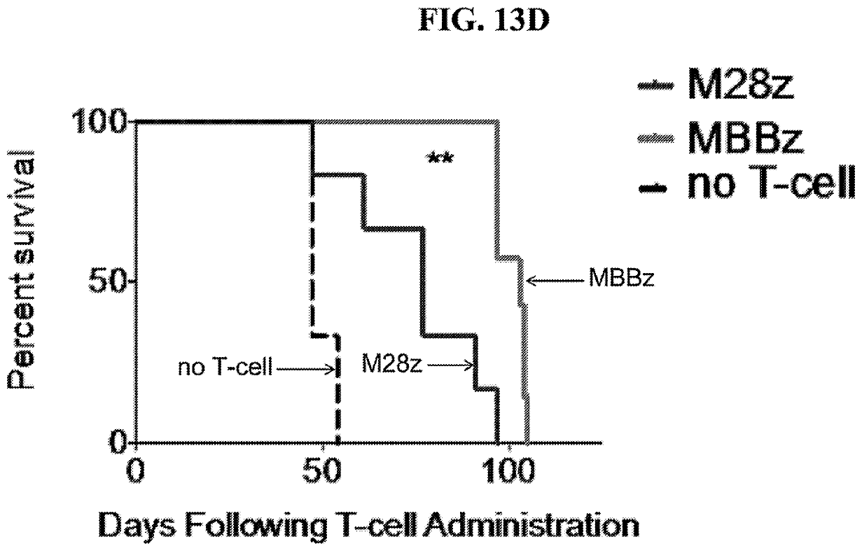

To confirm that the results were not limited to one cell line or mouse model, experiments were conducted to reproduce the results in a mouse model of metastatic lung cancer. The A549 lung cancer cell line was used, which expresses PD-L1 in vivo following M28z CAR T-cell therapy (FIG. 13A) as well as both PD-1 ligands following in vitro treatment with IFN-γ (FIG. 13B). Similar to the results of the mouse model of orthotopic mesothelioma, a single low dose of MBBz CAR T cells was better able to control tumor burden (FIG. 13C) and prolong tumor-free survival (median survival, 103 days for MBBz vs. 73 days for M28z CAR T cells) (FIG. 13D).

These results demonstrate that MBBz CAR T cells prolong tumor-free survival in a mouse model of metastatic lung cancer in which PD-1 receptor and complementary ligands are expressed.

### 7.11 Effect of PD-1 Dominant Negative Receptor (DNR) and PD-1 Switch Receptor on Tumors

As described above, cells expressing 4-1BB mesothelin CARs (MBBz) retain functional efficiency better than CD28 mesothelin CARs (M28z) when subjected to repeated antigen stimulation, as they are relatively resistant to PD1/PDL1-2 induced inhibition. PD1 DNR transduced into cells expressing M28z retain functional persistence due to the DNR.

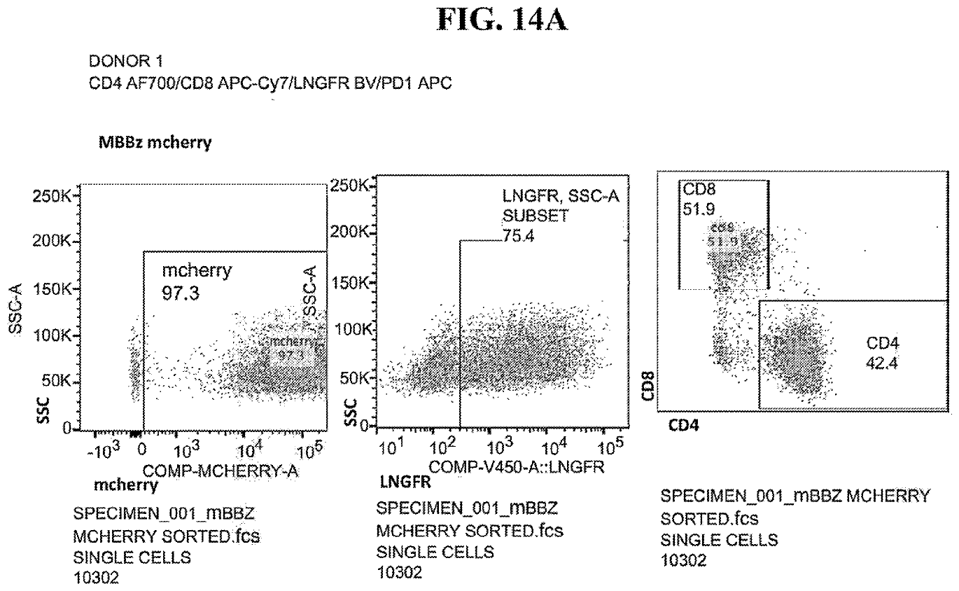

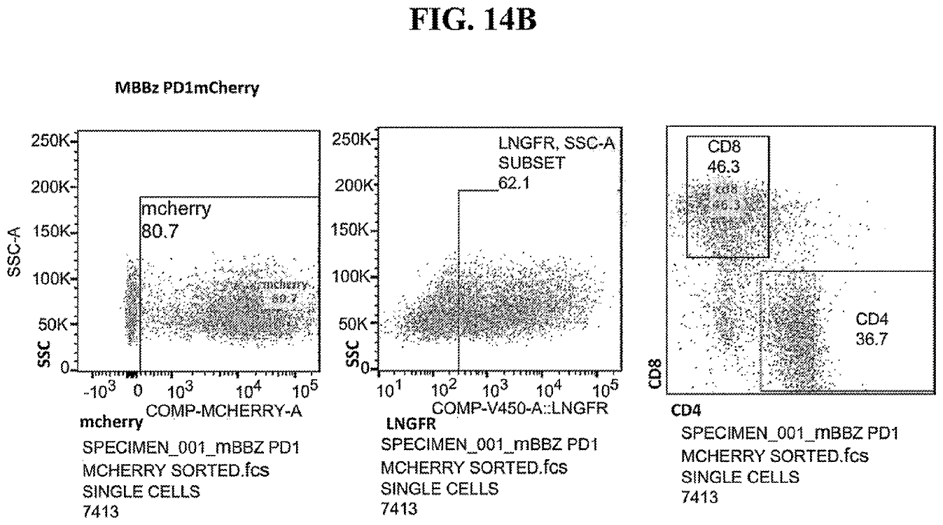

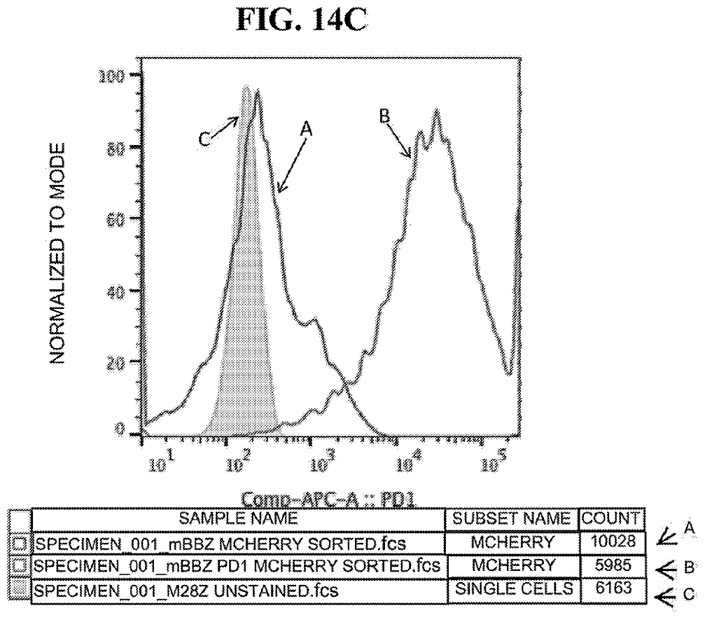

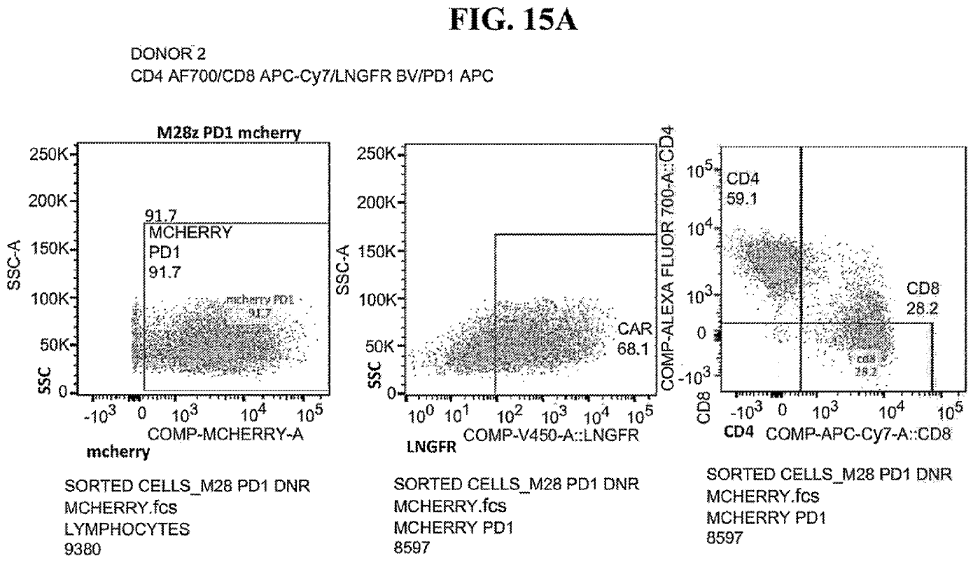

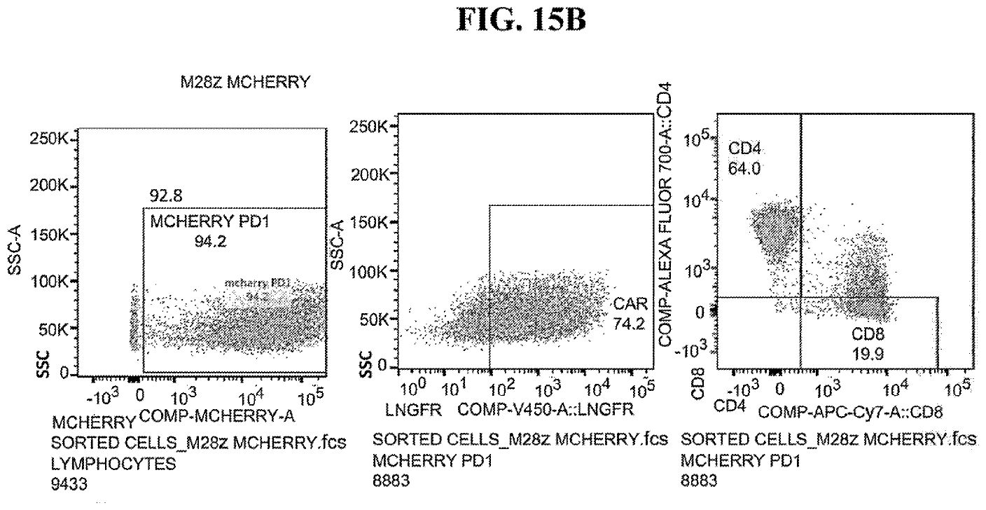

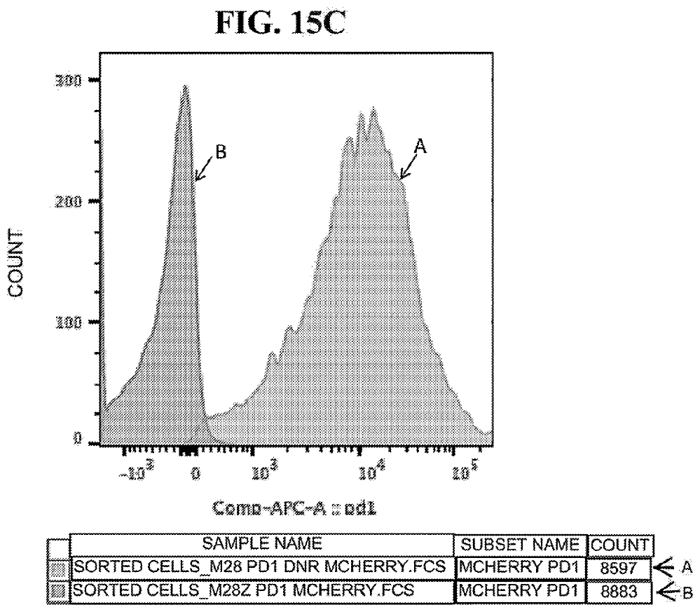

In order to further characterize the effect of PD1 DNR in CAR T cells, PD1 DNR was transduced into cells expressing MBBz CARs, with the expectation that the transduction would increase their efficiency even further, as seen with cells expressing M28z. To test PD1 DNR transduction into MBBz transduced CAR T cells, T cells from two human donors were used. Human T cells were isolated and transduced with MBBz or MBBzPD1DNR CAR, both with a mcherry marker to identify CAR transduced T cells. The transduced cells were analyzed by FACS analysis essentially as described above. The results for donor 1 are shown in FIGS. 14A-14C. Both CARs were successfully transduced at 62-75% transduction efficiency in both CD4 and CD8 T cells. Staining with PD-1 antibody showed the transduction of PD1 DNR in T cells (FIG. 14C). A similar analysis was performed on cells from donor 2, except that M28z was the CAR used. The results for donor 2 are shown in FIGS. 15A-15C. Both CARs were successfully transduced at 68-74% transduction in both CD4 and CD8 T cells. Staining with PD-1 antibody showed the transduction of PD1 DNR in T cells (FIG. 15B). These results show that PD1 DNR can be effectively transduced into MBBz transduced CAR T cells with 50-60% efficiency with double transduction of CAR and PD1 DNR, similar to the transduction efficiency seen with M28z CAR co-transduction with PD1 DNR.

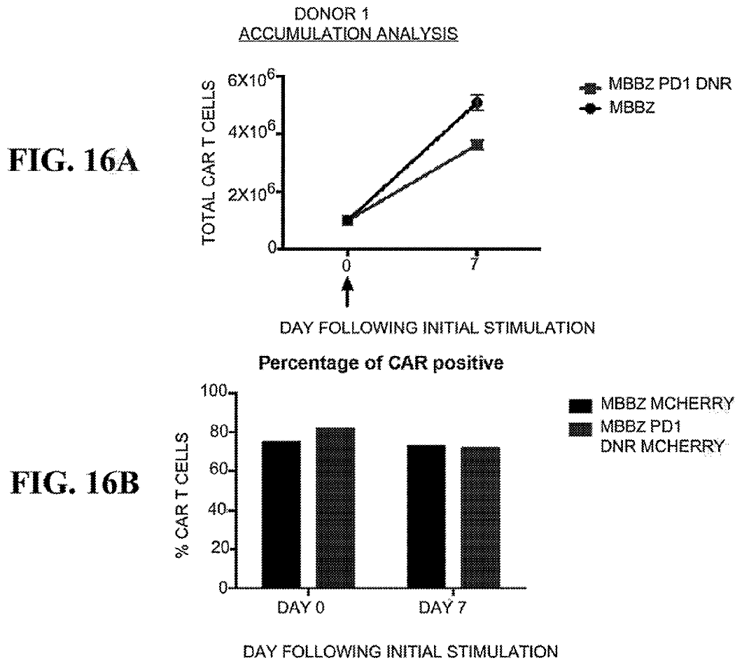

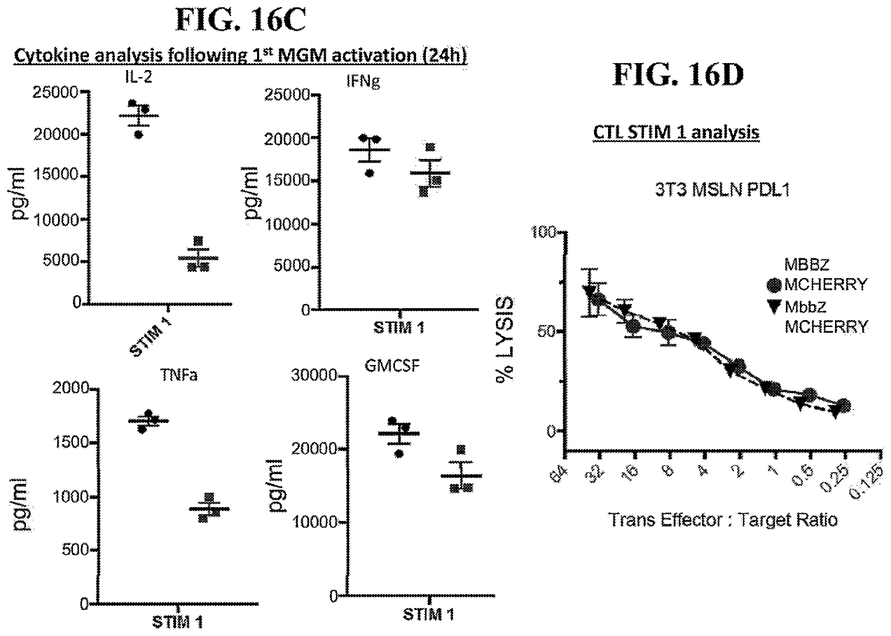

The efficacy of cells transduced with MBBz versus MBBz PD1 DNR was tested in vitro. Human T cells were isolated from two donors. In human T cells isolated from donor 1, both MBBz and MBBz PD1DNR transduced cells were exposed to antigen-expressing (mesothelin) targets and analyzed for T-cell accumulation, cytokine secretion and cytotoxicity essentially as described above. As shown in FIGS. 16A-16D, although T-cell accumulation (FIG. 16A), CAR T-cell percentage (FIG. 16B) and cytotoxicity (FIG. 16D) remained the same between both CARs, cytokine secretion was relatively lower in MBBzPD1DNR transduced CAR T cells (FIG. 16C). The results are shown in FIGS. 16A-16D.

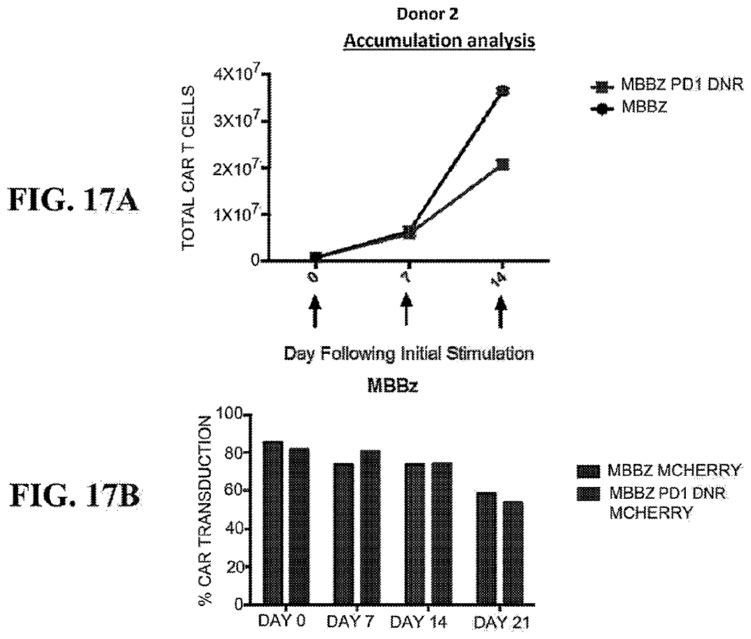

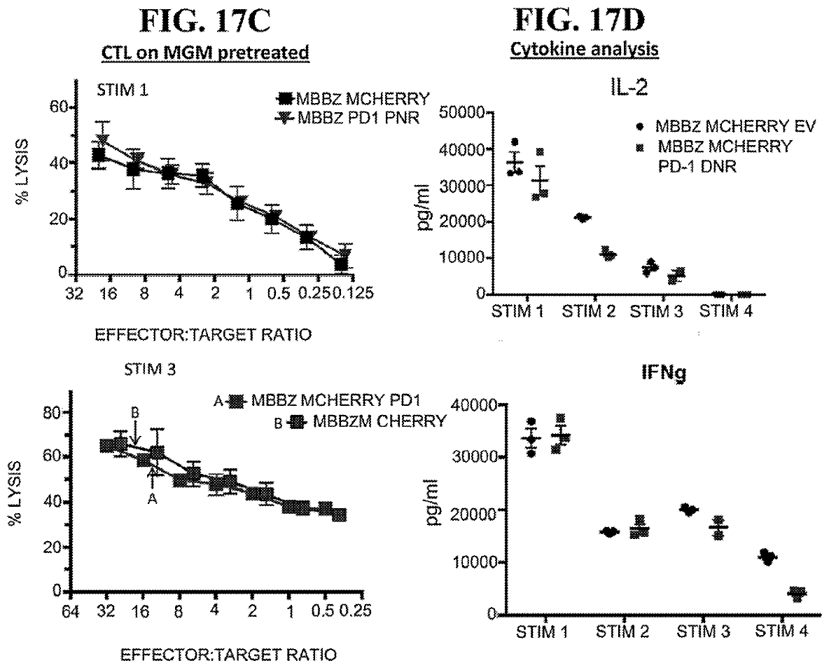

In cells isolated from donor 2, both MBBz and MBBz PD1DNR transduced cells were exposed to antigen-expressing (mesothelin) targets and analyzed for T-cell accumulation, cytokine secretion and cytotoxicity. In this experiment, the cytotoxicity assay was measured repeatedly after repeated antigen exposure. As shown in FIGS. 17A-17D, although T-cell accumulation (FIG. 17A), CAR T-cell percentage (FIG. 17B) and cytotoxicity (FIG. 17C) remained the same between both CARs, cytokine secretion (IL-2 and IFN-γ) was relatively lower in MBBzPD1DNR transduced CAR T cells (FIG. 17D). Upon repeated antigen exposure, although the cytotoxicity decreased, there were no differences between the two constructs (FIG. 17C).

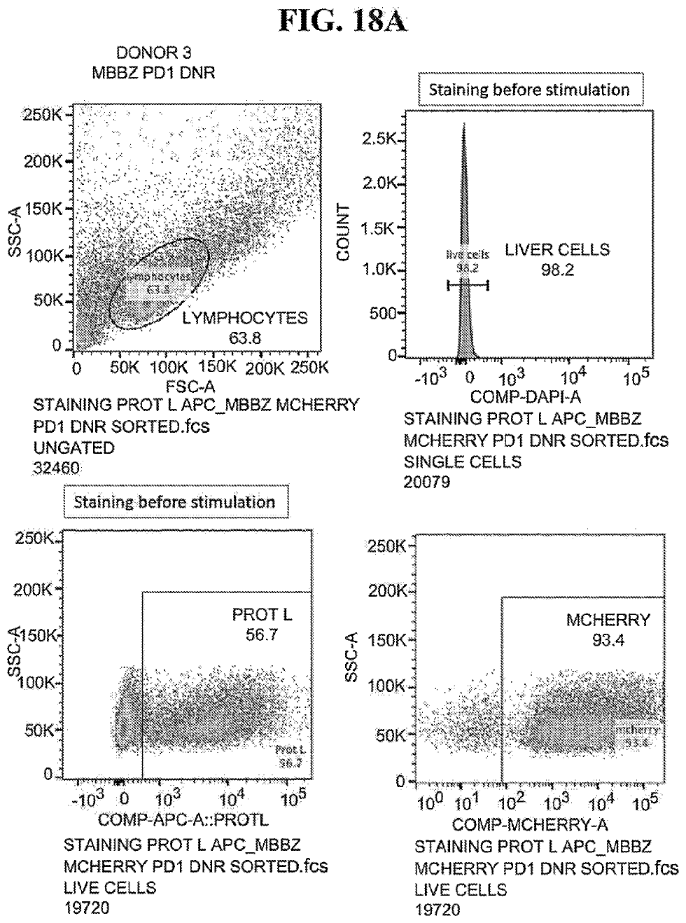

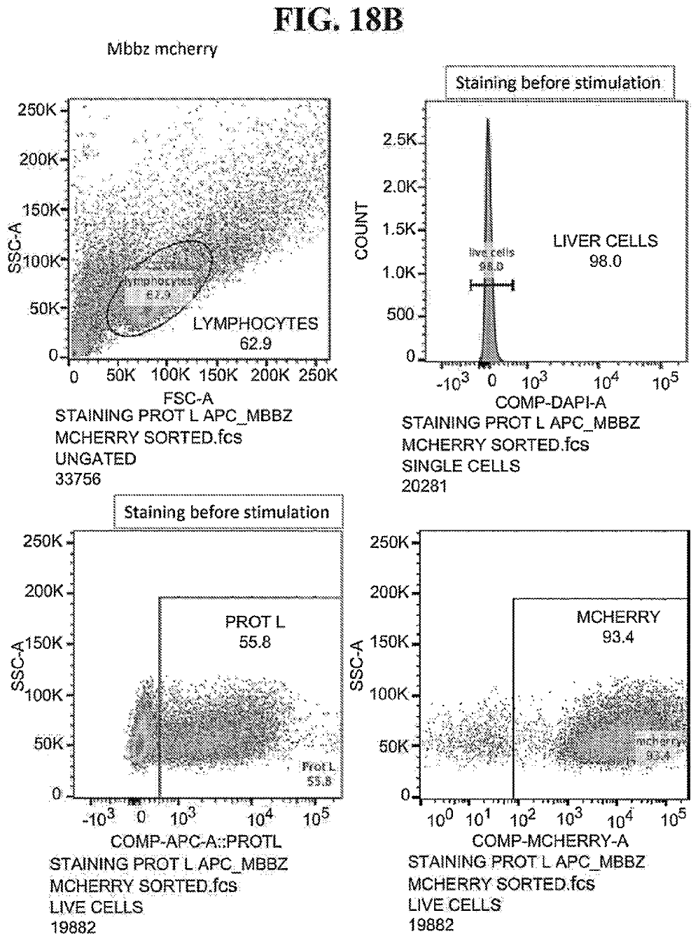

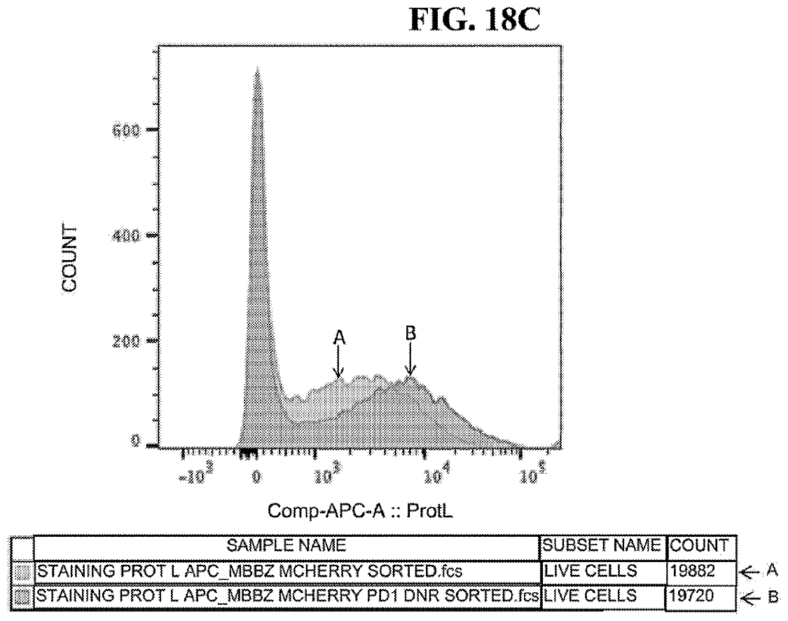

The effect of PD1 DNR transduction into MBBz transduced CAR T cells was tested. From donor 3, human T cells were isolated and transduced with MBBz or MBBzPD1DNR CAR, both with a mcherry marker to identify CAR transduced T cells. Cells were analyzed by FACS analysis, before and after stimulation. As shown in FIGS. 18A and 18B, both CARs were successfully transduced at 55-56% transduction efficiency in both CD4 and CD8 T cells. Staining with PD-1 antibody showed the transduction of PD1 DNR in T cells (FIG. 18B).

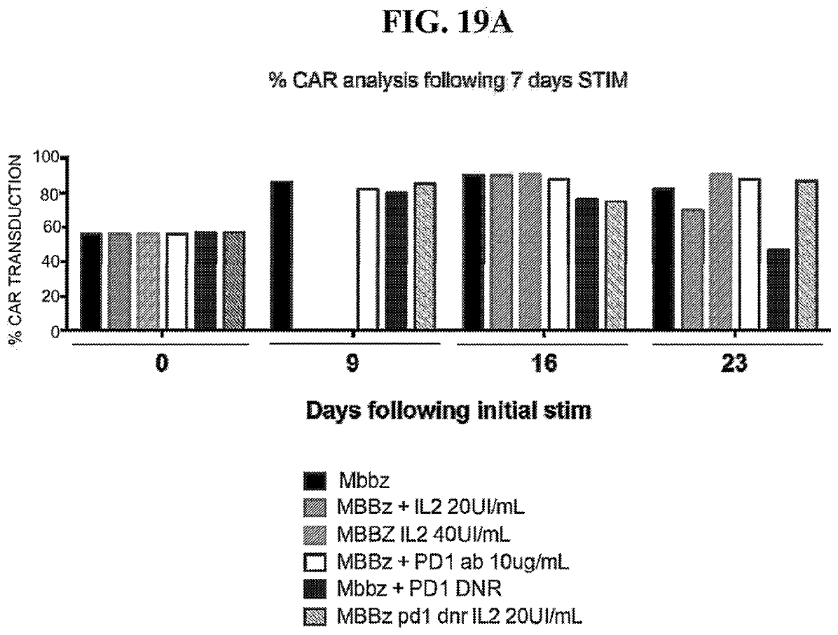

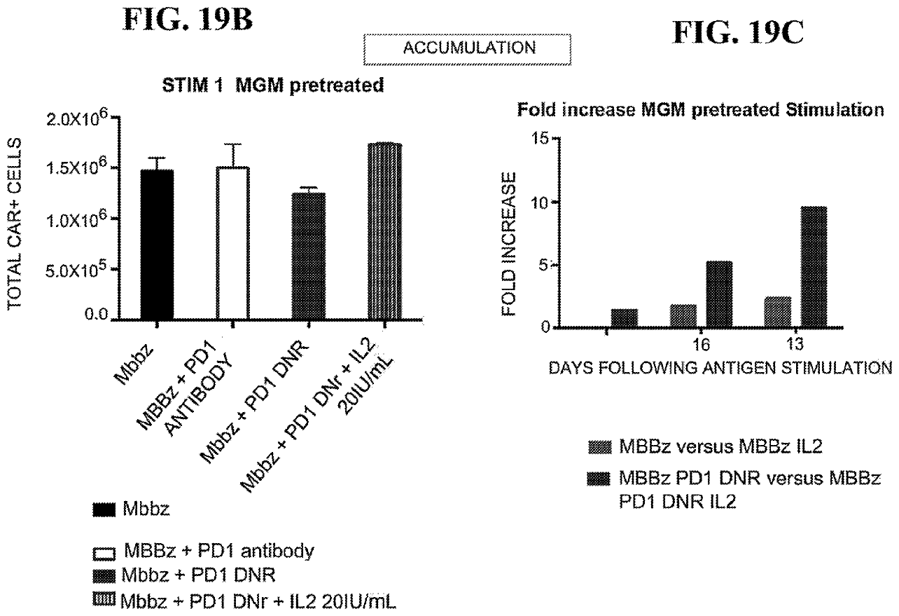

PD1 DNR transduction into MBBz transduced CAR T cells was further characterized. MBBz or MBBzPD1DNR CAR T cell accumulation was tested without or with IL-2 in the media. Cells transduced with MBBz or MBBz PD1 DNR were treated with or without 20 IU (international units)/mL IL-2, 40 IU IL-2/mL, or PD 1 antibody (10 μg/mL). Cells transduced with MBBz PD1 DNR were tested without or with 20 IU/mL IL-2. As shown in FIGS. 19A-19C, IL-2 supplementation rescued MBBzPD1DNR CAR T-cell accumulation upon antigen stimulation. These results show that IL-2 supplementation improved the effect of using the PD1 DNR transduced into MBBz transduced CAR T cells, unlike in M28z CAR T cells, where expression of PD1 DNR in M28z was effective without IL-2 (see above).

The results described above indicate that transduction of PD1 DNR into cells expressing MBBz CAR reduced the efficacy of the MBBz CAR T cells. Experiments were performed showing that unlike, M28z CARs that can produce higher amounts of IL-2, the ability of cells expressing MBBz CAR to secrete IL-2 is limited. Therefore, PD1 DNR transduction resulted in apoptosis. The effectiveness of MBBz PD-1 DNR T cells was rescued by addition of IL-2.

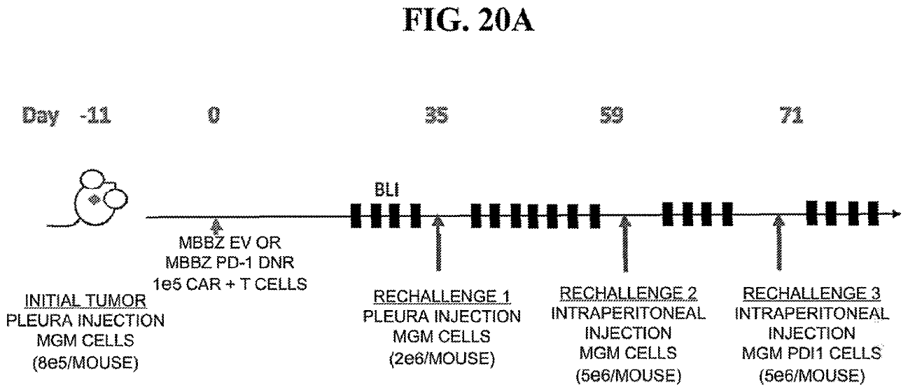

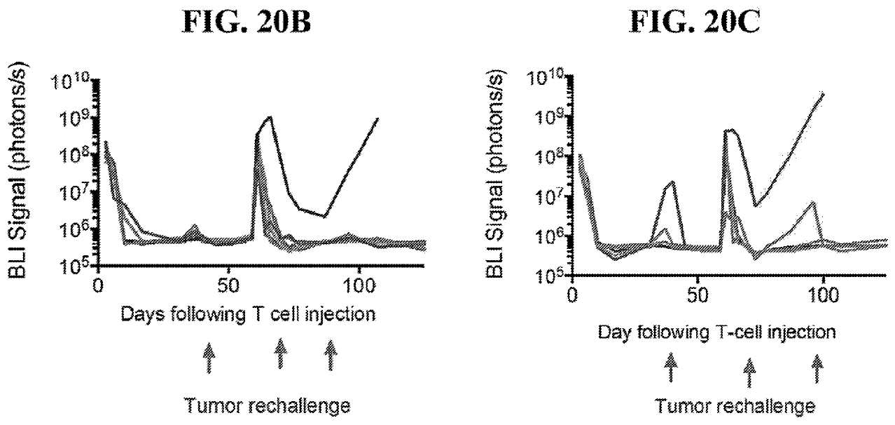

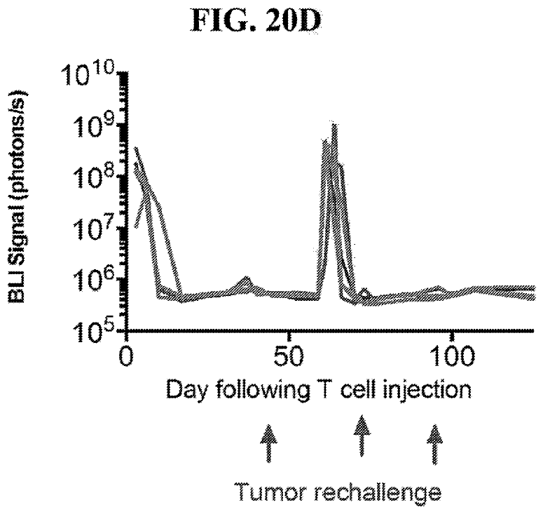

The efficacy of T cells expressing MBBz or MBBz PD1 DNR was examined in vivo. Mice with established pleurla tumor were treated with a single dose of MBBz or MBBzPD1DNR CAR T cells. Following tumor eradication, mice were rechallenged with either pleural or peritoneal tumor, and CAR T-cell functional persistence was assessed by tumor regression and eradication as assessed by BLI for the presence of tumor (FIG. 20A). As shown in FIGS. 20B-20D, three groups of mice (each group represented in a separate graph) were treated with a single low dose of cells expressing MBBz, cells expressing MBBz PD1 DNR, or cells expressing MBBz+PD1 blocking antibody. Each line in the graph indicates one mouse. Cells expressing both CARs successfully eradicated initial and subsequent rechallenge tumor burden with one small dose of CAR T cells. Both MBBz and MBBz PD1 DNR transduced CAR T cells effectively retained functional persistence in spite of 3 tumor rechallaneges, including one challenge with PD-L1 transduced cancer cells. The overall result is that mice in all 3 groups successfully eradicated tumor burden, even with multiple tumor rechallenges.

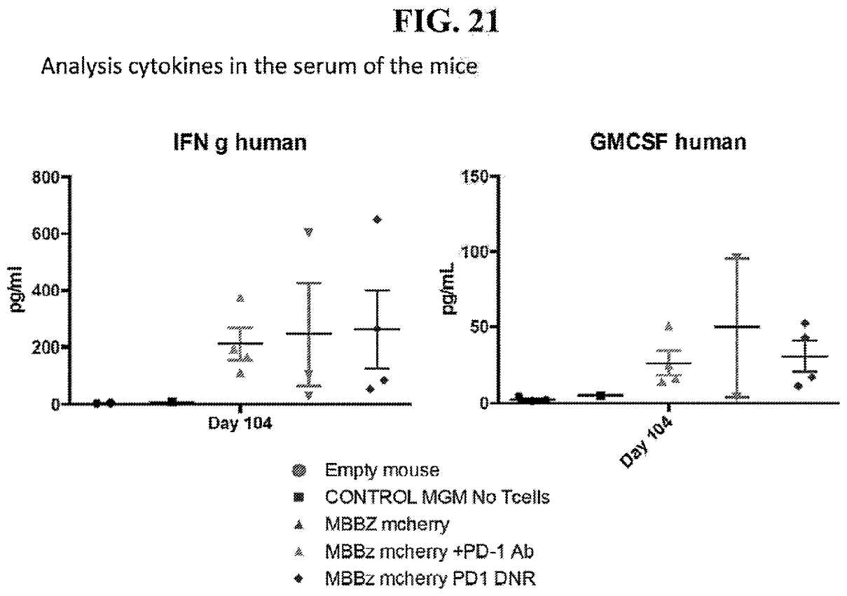

To assess the functional persistence of MBBz and mBBz PD1 DNR transduced CAR T cells, cytokines were analyzed in the serum of mice. As shown in FIG. 21, human interferon gamma and human GM-CSF in the serum of mice transduced with human T cells was assessed. Analysis of mouse serum following re-challenge at about 100 days (day 10⁴) shows the functional persistence of both MBBz and MBBz PD1 DNR transduced CAR T cells by detection of human cytokines (IFN-γ and GM-CSF) in mouse serum (FIG. 21).

The results described above indicate that differences between M28z and MBBz CARs when cotransduced with PD1 DNR can be identified, and therapies, such as additionally administering cytokines such as IL-2, can be utilized to improve the efficacy of immune cells expressing a CAR and immune checkpoint inhibitor dominant negative. Such results can be applied to clinical trial translation of the therapies.

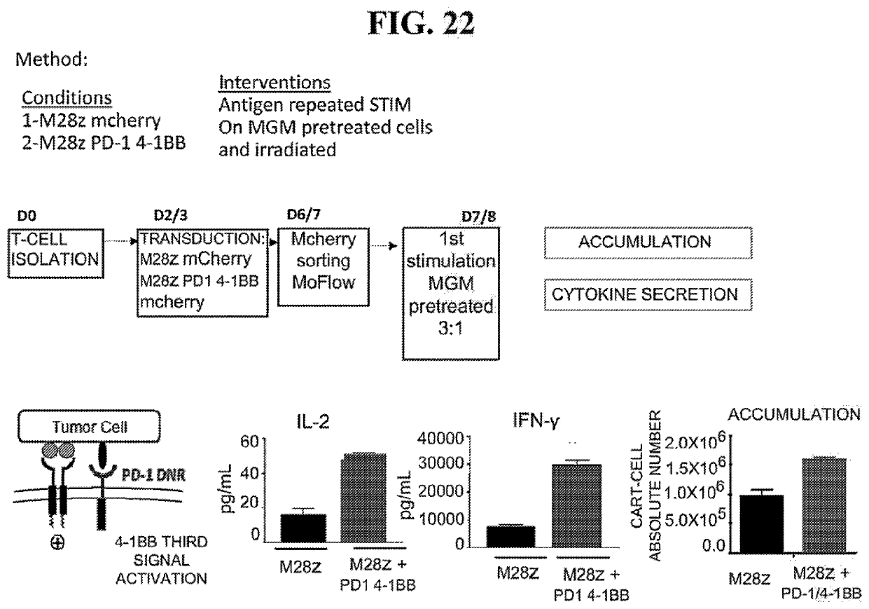

Experiments were also performed to strengthen M28z CAR T cell therapy. Human T cells were transduced with M28z or M28z PD1 4-1BB CAR, both with a mcherry marker, and were flow sorted and tested for cytokine secretion and T-cell accumulation. PD1 4-1BB is a “switch receptor” construct (see Liu et al., Cancer Res. 76:1578-1590 (2016)), with the extracellular PD-1 ligand binding domain fused to a transmembrane domain fused to the cytoplasmic signaling domain (co-stimulatory domain) of 4-1BB (shown schematically in FIG. 22). As shown in FIG. 22, T cells were isolated, and cells were transduced with M28z mcherry or M28z PD1 4-1BB mcheery on day 2 or 3. Cells were sorted by mcherry expression on day 5 or 6. On day 7 or 8, the first stimulation was initiated, MGM (mesothelin expressing cells) (see WO 2015/188141) pretreated (3:1). CAR T cell accumulation and cytokine secretion (IL-2 and IFN-γ) were measured essentially as described above. As shown in FIG. 22, PD1 4-1BB cotransduced CAR T cells showed higher cytokine secretion and accumulation compared to M28z transduced CAR T cells. PD1 4-1BB acted as a third signal to enhance the efficacy of cells transduced with M28z. The results show that, to rescue the PD-1/PD-L1 mediated inhibition, PD-1 4-1BB cotransducted into M28z CAR T cells induced a third stimulation, via PD-1 4-1BB following PD-L1 engagement, thus increasing M28z potency.

7.12. Converting Tumor-mediated PD-L1 Inhibition into CAR T-cell Costimulation to Potentiate Thoracic Cancers Immunotherapy

To overcome tumor-mediated inhibition of chimeric antigen receptor (CAR) T cells, the impact of tumor PD-L1 upregulation on CAR T-cell exhaustion and anti-tumor efficacy was investigated. In addition, experiments were performed to further develop clinically translatable T-cell extrinsic as well as intrinsic strategies to overcome PD-L1 inhibition in models of lung cancer (LC) and malignant pleural mesothelioma (MPM).

Human T cells were transduced with MSLN-specific CAR with CD28 and CD3zeta domains (M28z) and were tested in vitro and in clinically-relevant LC and MPM mouse models by bioluminescence imaging (BLI) of tumor burden progression. To counteract PD-1/PD-L1 inhibition in vivo, the efficacy of PD-1 blocking antibody or cell-intrinsic genetic-engineering strategies were evaluated by cotranducing M28z CAR T cells with a PD-1 dominant negative receptor (PD1-DNR) or with PD-1/4-1BB fusion protein.

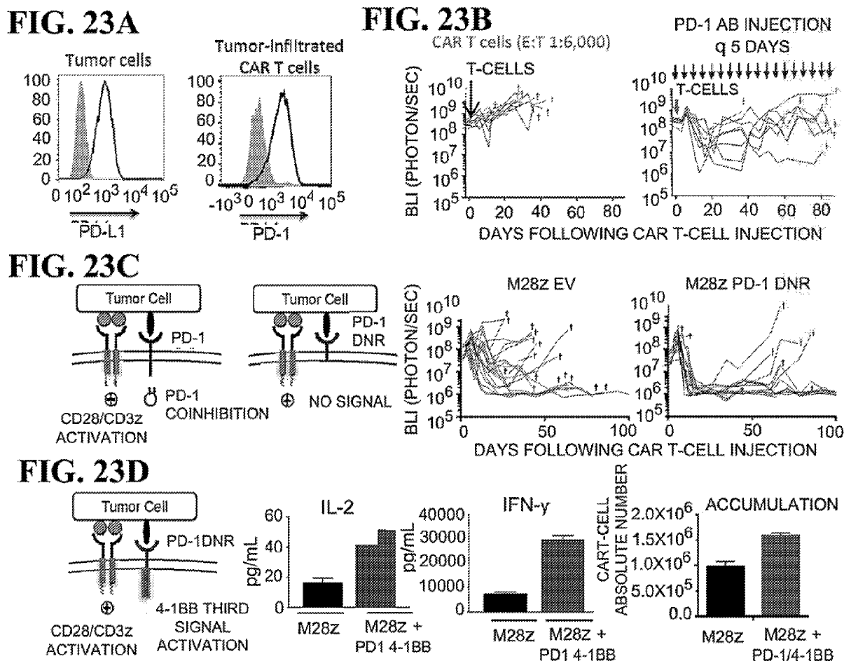

A single, low-dose of M28z CAR T cells was able to resist the progression of established tumor for 40 days, but mice eventually died with progressing tumor. Tumor harvest analysis demonstrated the PD-1 and PD-L1 upregulation on CAR T cells and tumor cells (FIG. 23A). It was then confirmed in vitro that PD-L1 inhibits M28z T-cell effector functions (proliferation, cytotoxicity and cytokine secretion).

The ability of a PD-1-blocking antibody (clone EH12.2H7) to rescue M28z CAR T cells was evaluated in vivo. For this purpose, a single, very low dose of M28z CAR T cells (5×10⁴, E:T ratio, 1:6,000) was injected into mice with large established tumor burdens with the objective of inducing the exhaustion of CAR T cells. In these conditions, CAR T cells were able to stabilize the tumor for 30 days (FIG. 23B). At day 30, the PD-1 antibody was administered intraperitoneally at 10 mg/kg 3 times every 5 days (Curran et al., Proc. Natl. Acad. Sci. USA 10⁷(9):4275-4280 (2010); Moon et al., Clin. Cancer Res. 22(2):436-447 (2016); Seung et al., PLoS ONE, 8(10):e77780 (2013)). There was a marked decrease in tumor BLI following 3 doses of the antibody. However, tumor relapses observed following cessation of treatment suggest that efficacy is short lived and reliant upon repeated PD-1 antibody administration. The addition of PD-1 blocking potentiates CAR T-cell therapy in vivo, but its efficacy requires multiple injections (FIG. 23B). As shown in FIG. 23B, multiple, long-term injections of the PD-1 antibody are able to control tumor burden but unable to eradicate the tumor.

In contrast to the results described above using the PD-1 blocking antibody, a single dose of M28z T cells coexpressing PD1-DNR restored effector functions and enhanced tumor burden control (FIG. 23C). This experiment is also described above, with the schematic diagram and the results of FIG. 23C shown in FIGS. 8A and 8E, respectively. A single dose of M28z T cells coexpressing PD1-DNR also prolonged median survival (56 vs 82 days, p=0.001).

PD-L1 inhibition was converted into a positive costimulatory signal by a PD-1/4-1BB switch receptor construct cotransducted into M28z CAR T cells. These cells exhibited enhanced cytokine secretion (IL-2 and IFN-γ) and T-cell accumulation (FIG. 23D). This experiment is also described above and the results shown in FIG. 22.

These results demonstrated the therapeutic benefit of providing optimized costimulation and coinhibitory blockade to counteract PD-L1/PD-1 immunosuppression, thus potentiating CAR T-cell therapy for lung cancer and mesothelioma.

### 7.13. Overview and Discussion of Experimental Results

As described above, CAR T-cell therapy and PD-1 checkpoint blockade have been demonstrated to be a rational combination in a solid tumor model. In vitro and ex vivo stimulation assays were performed to assess the impact of PD-1/PD-L1 inhibition on mesothelin CAR T-cell function. To directly counteract PD-1-mediated inhibition, retroviral vectors were used to combine CAR-mediated costimulation with a PD-1 DNR. Optimal signaling provided by this combinatorial strategy (costimulation and checkpoint blockade) enhanced T-cell function in the presence of tumor-encoded PD-L1 expression, resulting in long-term tumor-free survival following a single low dose of CAR T cells. These studies are relevant to the clinical practice of adoptive T-cell therapy and are immediately translational for the following reasons: (1) the costimulatory signaling domains tested-CD28 and 4-1BB are the two costimulatory domains used in ongoing clinical trials (NCT02414269, NCT02159716, NCT01583686), (2) the models of pleural mesothelioma recapitulate human disease and uses large, clinically relevant tumor burdens that elucidate the relevance of T-cell exhaustion (Adusumilli et al., Science Translational Medicine 6(261):261ra1 51 (2014); Servais et al., Clin. Cancer Res. 18(9):2478-2489 (2012); Servais et al., in Current Protocols in Pharmacology, Enna, ed., Chapter 14 (Unit14 21), John Wiley & Sons (2011); Servais et al., PLoS One 6(10):e26722 (2011)), and (3) the strategy of potentiating CAR T cells by genetically encoded checkpoint blockade uses human sequences that can be readily applied in the clinic (Adusumilli et al., Science Translational Medicine 6(261):261ral51 (2014); Feng et al., Mol. Cancer Ther. 8(5):1113-1118 (2009)).

The studies described above demonstrate that even T cells expressing second generation CARs are inhibited upon in vivo antigen exposure within the tumor microenvironment. That several other studies report that costimulation alone can overcome tumor-expressed inhibitory signaling may be explained by their reliance on in vitro studies, their use of immuno sensitive in vivo models, and their administration of high T-cell doses that do not reflect the burdens of established solid tumors seen in patients (Carter et al., Eur. J Immunol. 32(3):634-643 (2002); Freeman et al., J Exp. Med. 192(7):1027-1034 (2000); Koehler et al., Cancer Res. 67(5):2265-2273 (2007)). In the experiments described above, higher T-cell doses result in tumor eradication regardless of a CD28 or 4-1BB costimulatory domain. It is at the lower T-cell doses (and resulting lower effector:target ratios) that the effect of exhaustion becomes apparent. These findings illustrate the importance of using clinically relevant in vivo models and T-cell doses that are similar to those used in patient trials. The intrapleural T-cell doses used in the studies described above (4×10⁴ to 1×10⁵ per mouse equivalent to 1.2×10⁵ to 3×10⁶/Kg in human) are markedly lower doses than used in other mesothelioma xenografts studies (Carpenito et al., Proc. Natl. Acad. Sci. USA 10⁶(9):3360-3365 (2009); Zhao et al., Cancer Res. 70(22):9053-9061 (2010)) and is comparable to doses used in current clinical trials for hematologic malignancies (Brentjens et al., Science Translational Medicine 5(177):177ra38 (2013); Grupp et al., N. Engl. J Med. 368(16):1509-1518 (2013)) and solid tumors (Louis et al., Blood 118(23):6050-6056 (2011); Beatty et al., Cancer Immunol. Res. 2(2):112-120 (2014)). Therefore, the experimental strategy is particularly suited to characterize the role of exhaustion in CAR T-cell therapy.

In the results described above, although both 4-1BB and CD28 costimulatory signaling enhanced T-cell persistence to a similar degree, at lower E:T ratios, only treatment with 4-1BB-costimulated T cells eradicated tumor. 4-1BB-costimulated T cells, while still sensitive to tumor-mediated inhibition, were relatively resistant to decline in T-cell cytolytic function and cytokine secretion both following in vivo antigen exposure and upon repeated antigen stimulation in vitro. The resistance of 4-1BB signaling to immuno inhibition is associated with a more potent phenotype (PD-1^{lo}Tbet^{hi}, Eomesoderminw) (Curran et al., J. Exp. Med. 210(4):743-755 (2013); Hirschhorn-Cymerman et al., J. Exp. Med. 209(11):2113-2126 (2012); Song et al., Oncoimmunology 3(1):e27680 (2014); Schietinger et al., Science 335(6069):723-727 (2012); Kao et al., Nat. Immunol. 12(7):663-671 (2011)), which has been linked to less exhaustion and a more robust cytotoxic effector response in other tumor models and the analogous model of chronic viral infection. This suggests that the criteria for selecting a particular costimulatory signaling strategy among the options available, that is, 4-1BB, CD28, OX40L, 4-1BBL, CD27, and the like, should extend beyond T-cell persistence to “functional persistence,” which is the ability of T cells to function upon repeated antigen stimulation either initially within the tumor microenvironment or as may occur upon antigen rechallenge after control of primary tumor burden. As with previous studies supporting regional CAR T-cell therapy (Adusumilli et al., Science Translational Medicine 6(261):261ra151 (2014)), administering T cells with high functional persistence allows for single administrations of low T-cell doses, which can serve to limit cytokine release syndromes yet still eradicate primary tumor. It is important to note that these experiments do not mean that 4-1BB is the defacto costimulation agent to be used for patient therapy. The superior signaling pathway will depend on the unique patterns of costimulatory and coinhibitory ligand expression by the tumor, the antigen expression level or density, the affinity of scFv for the tumor antigen, the distance of the tumor epitope from the membrane, and variations in construct design (such as spacer and transmembrane domains) (Sadelain et al., Cancer Discovery 3(4):388-398 (2013); James et al., J. Immunol. 180(10):7028-7038 (2008); James et al., J. Immunol. 184(8):4284-4294 (2010); Watanabe et al., J. Immunol. 194(3):911-920 (2015); Hombach et al., J. Immunol. 178(7):4650-4657 (2007); Chmielewski et al., J Immunol. 173(12):7647-7653 (2004)). These variables, and not qualitative differences in signaling, may ultimately explain the variability seen in preclinical trials, which alternately conclude that 4-1BB or CD28 is superior, depending on the context. Indeed, the 4-1BB and CD28 constructs used in the experiments described above are sufficiently different in their transmembrane domains that conclusions determining the optimal costimulatory domain should not be made from these results, but can be determined using models such as those described above.

The relatively higher expression of PD-1 in M28z CAR T cells led to the focus on CD28-stimulated CAR T cells. On the basis of this analysis, genetic strategies were pursued for counteracting PD-1 inhibitory signaling, such as generating a PD-1 dominant negative receptor (PD-1 DNR) and shRNAs targeting PD-1. When expressed at sufficient levels, the PD-1 DNR competes with the endogenous PD-1 receptor for binding PD-1 ligands (PD-L1 and PD-L2). CD28-costimulated T cells cotransduced with PD-1 DNR demonstrated enhanced in vitro T-cell functions and in vivo T-cell efficacy, suggesting PD-1 signaling as a significant mechanism by which tumor cells evade CAR T cells in the tumor model. Although only in vitro efficacy was demonstrated for PD-1-targeting shRNAs, the absence of in vivo efficacy is likely related to saturation of shRNA machinery by the high volume of PD-1 transcripts induced following multiple in vivo antigen encounters, a conclusion supported by the finding that PD-1 knockdown was significantly lower in vivo than in vitro. The findings described above point to the therapeutic usefulness of adoptively transferred T cells that are genetically engineered to resist tumor-mediated immune inhibition. A DNR that targets TGF-β has been validated in preclinical models and is currently being tested in clinical trials (Foster et al., J Immunother. 31(5):500-505 (2008); Bollard et al., Blood 99(9):3179-3187 (2002)).

Whereas others have combined T-cell therapy with PD-1-blocking antibodies either in vivo or in vitro, the addition of a genetic strategy for coinhibitory blockade described in the experiments above overcomes several major obstacles limiting antibody therapy, including (1) the reliance on repeated administrations of antibodies and (2) the incidence of immune-related adverse events. T-cell therapy, then, has advantages over antibody therapy because it can establish long-term engraftment of T cells programmed for resistance to inhibition after a single dose and because it provides blockade of inhibitory pathways that is limited to a tumor-targeted T-cell repertoire, which may limit the autoimmunity that results from a more broadly applied antibody checkpoint blockade. Furthermore, it is possible that perhaps PD-L1 blocking antibodies can further prolong the efficacy of M28z and M28z PD-1 DNR CAR T cells.

The studies described above are unique when compared to other reports characterizing CAR T-cell exhaustion. Moon et al. characterized T-cell hypofunction within an immunoresistant mesothelioma tumor (Moon et al., Clin. Cancer Res. 20(16):4262-4273 (2014)); however, their characterization of inhibition rested on ex vivo experiments and they did not demonstrate a therapeutic strategy that enhances survival in vivo. In contrast, the studies described above confirm the presence of PD-1 mediated inhibition in vivo and demonstrate gene-engineered checkpoint blockade that can be employed in clinical settings. Long et al. recently described CAR T-cell exhaustion in a model of osteosarcoma (Long et al., Nat. Med. 21(6):581-590 (2015)). Their characterization, however, is fundamentally different in that they describe an antigen-independent phenomenon that results from tonic signaling of aggregated CAR receptors. The T cells in this model of Long et al., supra, become exhausted during ex vivo expansion, even prior to T-cell transfer. The results described above characterize a model of T-cell exhaustion more akin to that developed in the chronic viral infection literature, in which T-cell exhaustion is antigen-dependent and results from exposure to repeated antigen encounters in an environment rich with inhibitory signaling (Barber et al., Nature 439(7077):682-687 (2006); Mueller et al., Proc. Natl. Acad. Sci. USA 10⁶(21):8623-8628 (2009)).

The studies described above have identified one of the inhibitory mechanisms responsible for CAR T-cell and highlighted differences in the ability of costimulatory strategies to withstand immuno inhibition. Other inhibitory pathways may also function to potentially limit T-cell function. That a proportion of mice treated with PD-1 DNR-cotransduced M28z CAR T cells died of tumor progression suggests the action of other inhibitory mechanisms. Furthermore, the literature on chronic infection suggests the existence of other mechanisms of inhibition, both cell intrinsic and cell extrinsic, which are being assessed in tumor-targeted T-cell therapies (Moon et al., Clin. Cancer Res. 20(16):4262-4273 (2014); Riese et al., Cancer Res. 73(12):3566-3577 (2013)). Additional studies on inhibitory signaling can use an immunocompetent model that includes elements such as myeloid-derived suppressor cells and endogenous T cells, which have been shown to play important roles in tumor immune evasion.

The results described above have established the importance of tumor-mediated inhibition of CAR T-cell effector functions. By performing a comprehensive analysis of T-cell effector functions, it has been established that even costimulated CAR T cells, although they demonstrate enhanced persistence, are subject to inhibition upon repeated antigen encounter, both in vitro and within the tumor microenvironment. The results described demonstrate that CAR T-cell therapy can be used to counteract inhibitory signaling and provides the flexibility to engineer signaling domains that provide optimal costimulation and directly counteract inhibitory signals such as PD-1. Furthermore, in ongoing CAR T-cell therapy clinical trials in patients who show T-cell infiltration but a limited clinical response, combining PD-1/PD-L1 blockade following CAR T-cell therapy can be utilized to improve the efficacy of CAR T-cell therapy.

The results described above also show that the effectiveness of an immune cell expressing a CAR and a dominant negative form of an immune checkpoint inhibitor can be enhanced for immunotherapy. For example, the effectiveness of a T cell expressing a CAR and PD-1 DNR was increased by administering a cytokine, IL-2. The administration of IL-2 was found to be effective when the immune cell expressing a CAR and PD-1 DNR was deficient in producing IL-2.

The transcription factor nuclear factor of activated T cells (NFATc), upon activation of T cells through the T cell receptor, becomes dephosphorylated and translocates to the nucleus in lymphocytes (Serfling et al., Science Signaling (Sci. STKE) 398:pe42 (2007)). The translocated NFATc targets the IL-2 promoter. Induction of the IL-2 promoter in T cells depends critically on the activity of NFATc factors (Serfling et al., supra, 2007). A threshold abundance of NFAT factors needs to be reached in order for the induction of the Il-2 promoter (Serfling et al., supra, 2007). In both CD8 and CD4 T cells, PD1 induction following TCR stimulation requires NFATc (Bally et al., J. Immunol. 194:4545-4554 (2015)).

Unlike CD28 costimulation, which induces strong NF-κB, AP-1 and NFAT activity, 4-1BB costimulation reduces NFAT activity (Jutz et al., J Immunol. Methods 430:10-20. doi: 10.1016/j.jim.2016.01.007 (2016)). PD-1 strongly reduces NFAT activity (Jutz, supra, 2016)). 4-1BB signaling is mediated by TRAF2, which in turn inhibits NFAT-mediated transcription via NFAT-interacting protein NIP45 (Jutz et al., supra, 2016)). While not being bound by theory, it is possible that MBBz and PD-1 DNR reduced NFAT activity in MBBz PD1 DNR CAR cells, thereby reducing IL-2 production, decreasing T-cell proliferation, and increasing apoptosis (Serfling et al., Science Signaling (Sci. STKE) 398:pe42 (2007); (Bally et al., J. Immunol. 194:4545-4554 (2015); (Jutz et al., J. Immunol. Methods 430:10-20. doi: 10.1016/j.jim.2016.01.007 (2016)), unlike in cells expressing M28z PD-1 DNR CARs, which have abundant NFATc. 4-1BB signaling synergizes with PD-L1 blockade to augment CD8 T cell responses, but only at low or single dose combinations (Vezys et al., J. Immunol. 187:1634-1642 (2011)). Excessive usage of both results in decreased proliferation and increased apoptosis (Vezys et al., supra, 2011).

The effectiveness of an immune cell expressing a CAR and a dominant negative of an immune checkpoint inhibitor can also be enhanced by expression of a switch receptor, in which an intracellular signaling domain is fused to the extracellular ligand binding domain of an immune checkpoint inhibitor, such as PD-i. The results described above show that expression of a PD-1 extracellular domain fused to the cytoplasmic domain of 4-1BB increased cytokine production of IL-2 and interferon-gamma, and increased accumulation of CAR T cells. Expression of a switch receptor in an immune cell expressing a CAR can improve the efficacy of the immune cell for immunotherapy. Immune cells expressing a CAR and a switch receptor can be administered, concurrently or sequentially, with immune cells expressing a CAR and a dominant negative form of an immune checkpoint inhibitor to enhance the effectiveness of immunotherapy using such immune cells expressing a CAR and DN form of an immune checkpoint inhibitor.

The knowledge acquired from the clinical trials and the strategies presented herein are highly valuable to improve immunotherapy methods using CAR T cells, which is particularly use for therapy of solid tumors. Thus, the results described above exemplify methods that can be applied in a clinical setting to improve the efficacy of CAR T-cell therapy.

### 8. References Cited

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. A cell that is an immune cell or precursor cell thereof, which cell recombinantly expresses (a) a chimeric antigen receptor (CAR), and (b) a dominant negative form of an inhibitor of a cell-mediated immune response of the immune cell, wherein the CAR binds to a cancer antigen.

2. The cell of claim 1, wherein the immune cell is a T cell.

3. The cell of claim 1 or 2, wherein the precursor cell is a hematopoietic stem or hematopoietic progenitor cell.

4. The cell of any one of claims 1-3, wherein the immune cell is a cytotoxic T lymphocyte (CTL).

5. The cell of claim 1, wherein the cell is a T cell.

6. The cell of claim 1, wherein the cell is a Natural Killer (NK) cell.

7. The cell of any one of claims 1-6, wherein the inhibitor of a cell-mediated immune response is an immune checkpoint inhibitor.

8. The cell of claim 7, wherein the immune checkpoint inhibitor is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160.

9. The cell of claim 8, wherein the immune checkpoint inhibitor is PD-1.

10. The cell of any one of claims 1-6, wherein the inhibitor of a cell-mediated immune response is transforming growth factor β (TGF-β) receptor.

11. The cell of any one of claims 1-10, wherein the cancer antigen is selected from the group consisting of mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-α and β (FRα and β), Ganglioside G2 (GD2), Ganglioside G3 (GD3), human Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Rα2), κ-light chain, kinase insert domain receptor (KDR), Lewis A (CA19.9), Lewis Y (LeY), L1 cell adhesion molecule (LlCAM), melanoma-associated antigen 1 (melanoma antigen family A1, MAGE-A1), Mucin 16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-1H, HERK-V, IL-11Rα, Latent Membrane Protein 1 (LM1P1), Neural cell-adhesion molecule (N-CAM/CD56), and Trail Receptor (TRAIL R).

12. The cell of claim 11, wherein the cancer antigen is mesothelin.

13. The cell of claim 11, wherein the cancer antigen is mesothelin and the inhibitor of a cell-mediated immune response is PD-1.

14. The cell of any one of claims 1-13, wherein the cell further recombinantly expresses a suicide gene.

15. The cell of claim 14, wherein the suicide gene comprises inducible Caspase 9.

16. A pharmaceutical composition comprising a therapeutically effective amount of the cell of any one of claims 1-15; and a pharmaceutically acceptable carrier.

17. A polypeptide comprising (a) at least a portion of an extracellular domain of an immune checkpoint inhibitor, said portion comprising the ligand binding region, and (b) a transmembrane domain; wherein the polypeptide is a dominant negative form of the immune checkpoint inhibitor.

18. The polypeptide of claim 17, wherein the transmembrane domain is derived from a polypeptide other than the immune checkpoint inhibitor.

19. The polypeptide of claim 17 or 18, which lacks the intracellular domain of the polypeptide.

20. The polypeptide of any one of claims 17-19, wherein the immune checkpoint inhibitor is a receptor selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160.

21. The polypeptide of claim 20, wherein the immune checkpoint inhibitor is PD-1.

22. The polypeptide of any one of claims 17-21, wherein the transmembrane domain is of a cell surface polypeptide of a T cell.

23. The polypeptide of claim 22, wherein the cell surface polypeptide is selected from the group consisting of CD3, CD4, CD8, CD28, 4-1BB, OX40, ICOS, CTLA-4, LAG3, 2B4 and BTLA.

24. The polypeptide of claim 23, wherein the cell surface polypeptide is CD8 or CD28.

25. The polypeptide of claim 21, wherein the amino acid sequence of said polypeptide consists of the extracellular domain of PD-1 fused to the transmembrane and hinge domains of CD8.

26. A nucleic acid encoding the polypeptide of any one of claims 17-25.

27. A vector comprising the nucleic acid of claim 26.

28. A cell comprising the polypeptide of any one of claims 17-25.

29. A cell comprising the nucleic acid of claim 26.

30. A cell comprising the vector of claim 27.

31. A T cell that recognizes and is sensitized to a cancer antigen, which T cell recombinantly expresses a dominant negative form of an inhibitor of a T cell-mediated immune response.

32. The T cell of claim 31, wherein the inhibitor of a T cell-mediated immune response is an immune checkpoint inhibitor.

33. The T cell of claim 32, wherein the immune checkpoint inhibitor is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160.

34. The T cell of claim 33, wherein the immune checkpoint inhibitor is PD-1.

35. The T cell of claim 31, wherein the inhibitor of a cell-mediated immune response is transforming growth factor β (TGF-β) receptor.

36. The T cell of any one of claims 31-35, wherein the cancer antigen is selected from the group consisting of mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-α and p (FRα and p3), Ganglioside G2 (GD2), Ganglioside G3 (GD3), human Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Rα2), κ-light chain, kinase insert domain receptor (KDR), Lewis A (CA19.9), Lewis Y (LeY), L1 cell adhesion molecule (LlCAM), melanoma-associated antigen 1 (melanoma antigen family A1, MAGE-A1), Mucin 16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-1H, HERK-V, IL-11Rα, Latent Membrane Protein 1 (LMP1), Neural cell-adhesion molecule (N-CAM/CD56), and Trail Receptor (TRAIL R).

37. The T cell of claim 36, wherein the cancer antigen is mesothelin.

38. The T cell of claim 36, wherein the cancer antigen is mesothelin and the inhibitor of a cell-mediated immune response is PD-1.

39. The T cell of any one of claims 31-38, wherein the cell further recombinantly expresses a suicide gene.

40. The T cell of claim 39, wherein the suicide gene comprises inducible Caspase 9.

41. A pharmaceutical composition comprising a therapeutically effective amount of the T cell of any one of claims 31-40; and a pharmaceutically acceptable carrier.

42. A method of treating a cancer in a subject in need thereof, comprising administering to the subject a therapeutically effective amount of the cell of any one of claims 1-15, wherein the cancer antigen is an antigen of the cancer.

43. A method of treating a cancer in a subject in need thereof, comprising administering to the subject the pharmaceutical composition of claim 16, wherein the cancer antigen is an antigen of the cancer.

44. A method of treating a cancer in a subject in need thereof, comprising administering to the subject a therapeutically effective amount of the T cell of any one of claims 31-40, wherein the cancer antigen is an antigen of the cancer.

45. A method of treating a cancer in a subject in need thereof, comprising administering to the subject the pharmaceutical composition of claim 41, wherein the cancer antigen is an antigen of the cancer.

46. The method of any one of claims 42-45, wherein the cancer is selected from the group consisting of mesothelioma, lung cancer, pancreatic cancer, ovarian cancer, breast cancer, colon cancer, pleural tumor, glioblastoma, esophageal cancer, gastric cancer, and synovial sarcoma.

47. The method of any one of claims 42-46, wherein the administering is by intrapleural administration, intravenous administration, subcutaneous administration, intranodal administration, intratumoral administration, intrathecal administration, intraperitoneal administration, intracranial administration, or direct administration to the thymus.

48. The method of any one of claims 42-46, wherein the cancer antigen is mesothelin, and the cancer is selected from the group consisting of mesothelioma, lung cancer, and breast cancer.

49. The method of claim 48, wherein the subject has malignant pleural disease.

50. The method of claim 48 or 49, wherein the cells are administered intrapleurally.

51. The method of any one of claims 42-50, wherein the subject has a tumor.

52. The method of claim 51, wherein tumor growth is inhibited.

53. The method of any one of claims 42-52, wherein the cell is administered in a dose in the range of 10⁴ to 10¹⁰ cells per kilogram of body weight.

54. The method of claim 53, wherein the dose is in the range of 3×10⁵ to 3×10⁶ cells per kilogram of body weight.

55. The method of any one of claims 42-54, wherein the subject is a human.

56. The cell of any one of claims 1-15 which is derived from a human.

57. The T cell of any one of claims 31-40 which is derived from a human.

58. The pharmaceutical composition of claim 16 or 41 wherein the cell is derived from a human.

59. The method of claim 55 wherein the cell is derived from a human.

60. The method of any one of claims 42, 43, 46-55 or 59, wherein the CAR comprises a co-stimulatory signaling domain.

61. The method of claim 60, wherein the co-stimulatory signaling domain is the intracellular signaling domain of 4-1BB.

62. The method of claim 61, wherein the method further comprises administering a cytokine to the subject.

63. The method of claim 62, wherein the cytokine is IL-2 or GM-CSF.

64. The method of claim 63, wherein the cytokine is IL-2.

65. The method of claim 60, wherein the method further comprises administering an immune cell recombinantly expressing the chimeric antigen receptor (CAR) and a switch receptor, wherein the switch receptor comprises (i) at least the extracellular ligand binding domain of an immune checkpoint inhibitor, (ii) a transmembrane domain, and (iii) a co-stimulatory signaling domain.

66. The method of claim 65, wherein the co-stimulatory signaling domain of the switch receptor is different from the co-stimulatory signaling domain of the CAR.

67. The method of claim 65 or 66, wherein the co-stimulatory signaling domain of the CAR is the intracellular signaling domain of CD28.

68. The method of claim 67, wherein the co-stimulatory signaling domain of the switch receptor is the intracellular signaling domain of 4-1BB.
